(19) United States
(12) Patent Application Publication
(10) Pub. No.: US 2024/0423664 A1
(43) Pub. Date: Dec. 26, 2024

(54) EXPANDABLE ACCESS SHEATHS

(71) Applicant: EDWARDS LIFESCIENCES CORPORATION, Irvine, CA (US)

(72) Inventors: Tyler Poon, Ocala, FL (US); Steven Park, Santa Ana, CA (US); Anne Bernadette Aragon Alcasid, Costa Mesa, CA (US); Michael G. Valdez, Riverside, CA (US)

(21) Appl. No.: 18/821,639

(22) Filed: Aug. 30, 2024

Related U.S. Application Data
(63) Continuation of application No. PCT/US2023/014574, filed on Mar. 6, 2023.
(60) Provisional application No. 63/335,696, filed on Apr. 27, 2022.
(60) Provisional application No. 63/317,508, filed on Mar. 7, 2022.

(51) Int. Cl. A61B 17/34; A61M 25/00; A61M 25/01

(52) U.S. Cl. CPC A61B 17/3423; A61B 17/3496; A61M 25/0074; A61M 25/0097; A61M 25/0102

(57) ABSTRACT

Introducers and dilators that include expansion features configured to dilate expandable access sheaths, and expandable access sheaths, are described herein. The introducer can include an expansion feature disposed on an elongate shaft. The expansion feature can include a larger outer diameter than the distal end of the elongate shaft or another portion of the elongate shaft. The expansion feature can be configured to dilate a portion of an expandable access sheath when the elongate shaft is translated within the expandable access sheath. Expandable access sheaths can comprise expandable distal end portions configured to be maintained in a closed state by, for example, a tensioned cord portion disposed around a circumferential portion of the expandable distal end portion, a plurality of pleats comprising material configured to attract one another, a circumferential portion with a weakened joint, and/or a weakened portion on a sidewall of the expandable distal end portion.

### CROSS REFERENCE TO RELATED APPLICATIONS

This application is a continuation of International Patent Application No. PCT/US2023/014574, filed Mar. 6, 2023, entitled “INTRODUCER WITH DILATION FEATURE AND EXPANDABLE ACCESS SHEATHS,” which claims the benefit of U.S. Provisional Patent Application No. 63/335,696, filed Apr. 27, 2022, entitled “INTRODUCER WITH DILATION FEATURE AND EXPANDABLE ACCESS SHEATHS,” and U.S. Provisional Patent Application No. 63/317,508, filed Mar. 7, 2022, entitled “INTRODUCER WITH DILATION FEATURE,” the disclosures of each of which are hereby expressly incorporated by reference herein in their entireties for all purposes.

### BACKGROUND

The present disclosure generally relates to the field of medical devices. Delivery catheters and other therapy devices are often used to deliver medical devices, such as prosthetic heart valves, shunts, stents, or other devices, to locations inside the body that are not readily accessible by surgery or where access without invasive surgery is desirable. An access sheath can often be used to introduce a therapy device into the desired anatomy, such as the patient's vasculature or other anatomy. In some instances, the access sheath may include a relatively large diameter that can cause damage to the patient's tissue when the access sheath is inserted into the tissue. Further, in some instances, the therapy device can be damaged when pushed through the access sheath. For example, the access sheath can include a smaller inner diameter than the outer diameter of the therapy device, requiring a relatively high push force to move the therapy device through the access sheath. This can damage a fragile therapy device.

### SUMMARY

Described herein are methods and devices relating to an introducer comprising an elongate shaft configured to be received within an expandable access sheath. The introducer can have an expansion feature disposed on the elongate shaft. The expansion feature can include a larger outer diameter than an inner diameter of at least a portion of the expandable access sheath, including a distal end portion of the expandable access sheath. The expansion feature can be configured to dilate at least a portion of the expandable access sheath, including the distal end portion of the expandable access sheath, such as when the elongate shaft is translated distally within the expandable access sheath.

Described herein are methods and devices relating to expandable access sheaths. In some instances, a medical access system can comprise an expandable access sheath comprising a shaft having an expandable distal end portion. The medical access system can include a cord comprising a medial cord portion between first and second cord tail portions, the medical cord portion being configured to be disposed around a circumferential portion of the expandable distal end portion. The first and second cord tail portions can be configured to pass through a side shaft opening and comprise respective proximal portions for manipulation by an operator to transform the expandable distal end portion to an open and/or closed state. In some instances, an expandable access sheath can have a shaft comprising an expandable distal end portion with a plurality of pleats. Each pleat can comprise a pair of pleat portions configured to be attracted to one another to maintain the expandable distal end portion in a closed state. In some instances, each pleat portion can comprise magnetic material and/or electrostatic charges. In some instances, an expandable access sheath can have a shaft with an expandable distal end portion comprising a circumferential portion with a weakened joint. The circumferential portion can be configured to separate along the weakened joint to allow the expandable distal end portion to transform from a closed state to an open state. In some instances, an expandable access sheath can have a shaft with an expandable distal end portion comprising a weakened portion formed in a sidewall while the expandable distal end portion is in a closed state. The weakened portion can have a distal end spaced from a distal end of the shaft. At least a portion of the weakened portion can have an orientation at an angle relative to the distal end of the shaft. The expandable distal end portion can be configured to separate along the weakened portion.

Methods and structures disclosed herein for treating a patient also encompass analogous methods and structures performed on or placed on a simulated patient, which is useful, for example, for training; for demonstration; for procedure and/or device development; and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof; synthetic; or any combination of natural and synthetic. Virtual elements can be entirely in silica, or overlaid on one or more of the physical components. Virtual elements can be presented on any combination of screens, headsets, holographically, projected, loud speakers, headphones, pressure transducers, temperature transducers, or using any combination of suitable technologies.

For purposes of summarizing the disclosure, certain aspects, advantages and features have been described herein. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

### BRIEF DESCRIPTION OF THE DRAWINGS

Various examples are depicted in the accompanying drawings for illustrative purposes and should in no way be interpreted as limiting the scope of the subject matter. In addition, various features of different disclosed examples can be combined to form additional examples, which are part of this disclosure. Throughout the drawings, reference numbers may be reused to indicate correspondence between reference elements. However, it should be understood that the use of similar reference numbers in connection with multiple drawings does not necessarily imply similarity between respective examples associated therewith. Furthermore, it should be understood that the features of the respective drawings are not necessarily drawn to scale, and the illustrated sizes thereof are presented for the purpose of illustration of various aspects thereof. For example, illustrated features may be relatively smaller/larger than as illustrated in some examples/configurations.

FIG. 1 illustrates an example introducer/access system/assembly according to one or more implementations.

FIG. 2 illustrates example details of the introducer from FIG. 1 according to one or more implementations.

FIG. 3A illustrates the example access sheath from FIG. 1 having a substantially straight shape and/or form according to one or more implementations.

FIG. 3B illustrates the example access sheath from FIG. 1 having a tapered shape and/or form according to one or more implementations.

FIG. 4A illustrates an example access sheath implemented as an elastically expandable sheath according to one or more implementations.

FIG. 4B illustrates the access sheath from FIG. 4A as an introducer is advanced through the access sheath according to one or more implementations.

FIG. 4C illustrates the access sheath from FIG. 4A as the introducer is advanced out the distal end of the access sheath according to one or more implementations.

FIG. 5A illustrates an example access sheath implemented as a permanently expandable sheath according to one or more implementations.

FIG. 5B illustrates the access sheath from FIG. 5A as an introducer is advanced through the access sheath according to one or more implementations.

FIG. 5C illustrates the access sheath from FIG. 5A as the introducer is advanced out the distal end of the access sheath according to one or more implementations.

FIG. 6A illustrates an example access sheath that includes a smaller and/or crimped inner diameter at the distal end of the access sheath according to one or more implementations.

FIG. 6B illustrates the access sheath from FIG. 6A as an introducer is advanced through the access sheath according to one or more implementations.

FIG. 6C illustrates the access sheath from FIG. 6A as the introducer is advanced out the distal end of the access sheath according to one or more implementations.

FIG. 7A illustrates a side view of an example locking mechanism, assembly and/or means to ensure that an introducer is axially translated before the introducer is removed from an access sheath according to one or more implementations.

FIG. 7B illustrates a cross-sectional view of the locking mechanism from FIG. 7A according to one or more implementations.

FIGS. 8-1, 8-2 and 8-3 illustrate an example flow diagram of a process for dilating and/or expanding an access sheath using an introducer in accordance with one or more implementations.

FIGS. 9-1, 9-2 and 9-3 are images of example anatomy and devices associated with the process of FIGS. 8-1, 8-2 and 8-3 to illustrate aspects of the process according to one or more implementations thereof.

FIG. 10 illustrates a cross-sectional view of an example advancement configuration of an introducer and access sheath according to one or more implementations.

FIGS. 11A and 11B are perspective views of an example of a medical access system comprising a shaft with an expandable distal end portion, and a cord positioned around the expandable distal end portion and configured to facilitate maintaining the expandable distal end portion in a closed state, according to one or more implementations.

FIGS. 12A and 12B are perspective views of an of another example of a medical access system comprising a shaft with an expandable distal end portion, and a cord positioned around the expandable distal end portion and configured to facilitate maintaining the expandable distal end portion in a closed state, according to one or more implementations.

FIGS. 13A and 13B are perspective views of an example of a medical access system comprising an introducer preloaded within an expandable access sheath, and a cord positioned around an expandable distal end portion of the expandable access sheath and configured to facilitate maintaining the expandable distal end portion in a closed state, according to one or more implementations.

FIGS. 14A and 14B are perspective views of another example of a medical access system comprising an introducer preloaded within an expandable access sheath, and a cord positioned around an expandable distal end portion of the expandable access sheath and configured to facilitate maintaining the expandable distal end portion in a closed state, according to one or more implementations.

FIG. 15 is a process flow diagram illustrating a process for delivering a medical device using a medical access system as described herein, according to one or more implementations.

FIGS. 16A and 16B are perspective views of an example of an expandable access sheath that includes a shaft comprising an expandable distal end portion having a plurality of pleats, where respective pairs of pleat portions of each of the pleats can be configured to be attracted to one another to maintain the expandable distal end portion in a closed state, according to one or more implementations.

FIGS. 17A and 17B are distal end views of an example of an expandable distal end portion, in a closed state and an open state, respectively, where the expandable distal end portion comprises a plurality of pleats, and respective pairs of pleat portions of each of the pleats can be configured to be attracted to one another, according to one or more implementations.

FIGS. 18A and 18B are distal end views of another example of an expandable distal end portion, in a closed state and an open state, respectively, where the expandable distal end portion comprises a plurality of pleats, and respective pairs of pleat portions of each of the pleats can be configured to be attracted to one another, according to one or more implementations.

FIG. 19 is a process flow diagram illustrating a process for delivering a medical device using an expandable access sheath as described herein, according to one or more implementations.

FIGS. 20A and 20B are perspective views of an example of an expandable access sheath that includes a shaft comprising an expandable distal end portion having a circumferential portion with a weakened joint, according to one or more implementations.

FIG. 21 is a process flow diagram illustrating a process for delivering a medical device using an expandable access sheath as described herein, according to one or more implementations.

FIGS. 22A and 22B are side views of an example of an expandable access sheath comprising a shaft with an expandable distal end portion that includes a weakened portion in a sidewall of the shaft, according to one or more implementations.

FIGS. 23A and 23B are side views of another example of an expandable access sheath comprising a shaft with an expandable distal end portion that includes a weakened portion in a sidewall of the shaft, according to one or more implementations.

FIGS. 24A and 24B are side views of another example of an expandable access sheath comprising a shaft with an expandable distal end portion that includes a weakened portion in a sidewall of the shaft, according to one or more implementations.

FIG. 25 is a process flow diagram illustrating a process for delivering a medical device using an expandable access sheath as described herein, according to one or more implementations.

### DETAILED DESCRIPTION

The headings provided herein are for convenience only and do not necessarily affect the scope or meaning of the subject matter.

Although certain examples are disclosed below, the subject matter extends beyond the specifically disclosed examples to other alternative examples and/or uses and to modifications and equivalents thereof. Thus, the scope of the claims that may arise here from is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be implemented as integrated components or as separate components. For purposes of comparing various examples, certain aspects and advantages of these examples are described. Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein.

Certain standard anatomical terms of location are used herein to refer to the anatomy of animals, and namely humans, with respect to the examples. Although certain spatially relative terms, such as “outer,” “inner,” “upper,” “lower,” “below,” “above,” “vertical,” “horizontal,” “top,” “bottom,” and similar terms, are often used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa.

An access sheath can be used to introduce a medical instrument/device into a patient's anatomy (e.g., a catheter or other delivery device into the patient, including the femoral artery or other anatomy). In some cases, at least a portion of an access sheath, such as an expandable access sheath, can be designed to expand from an initial state having a first inner diameter (ID) to an expanded state having a second larger ID. For example, the access sheath can be introduced into the desired anatomy in the initial state with the smaller ID. Once the access sheath is positioned, a medical instrument, such as a catheter or other delivery device, can be inserted through the access sheath to reach a target site within the patient. For example, the catheter or other delivery device can be advanced through the access sheath positioned into the patient to position the catheter or other delivery device at the target site within the patient. The access sheath can expand to the larger ID to accommodate the catheter or other delivery device. In some instances, the catheter or other delivery device can be configured to carry a therapy device. In some instances, the therapy device can be carried by and/or preloaded within the catheter or other delivery device. This can reduce, minimize, or prevent damage to the patient's anatomy and/or the access sheath (e.g., avoid damaging the tissue of the patient with a sheath having a traumatic point (relatively large diameter), avoid the access sheath from folding inward on itself during insertion of the access sheath, etc.).

In some solutions, a relatively high push force is needed to push the delivery device, including a delivery device carrying a therapy device, through the access sheath and/or out of the distal end of the access sheath. The push force can refer to the amount of effort or force exerted axially on the delivery device and/or therapy device, such as to effect expansion of the access sheath radially, to allow the delivery device and/or therapy device to travel through the access sheath axially. The push force can refer to the amount of effort or force exerted axially on the delivery device and/or therapy device to distally advance the delivery device and/or therapy device through the access sheath. However, some delivery devices and/or therapy devices may not be able to withstand excessive push forces (e.g., due to the relatively fragile form of the delivery devices and/or therapy devices), resulting in damage to the delivery devices and/or therapy devices. Further, excessive push force can cause the delivery device and/or therapy device to abruptly eject from the access sheath, causing damage to the patient.

In some instances, the present disclosure describes devices, systems, and methods relating to introducers that include expansion features configured to dilate access sheaths in a manner that reduces, minimizes, or prevents damage to the patient's anatomy, and/or access sheaths and/or medical devices that are used therewith. For example, an access sheath, such as an expandable access sheath, can be inserted into the patient's anatomy with a collapsed or relatively small profile and/or form. After the access sheath is positioned within the desired anatomy, the introducer can be translated through the access sheath to expand and/or stretch the access sheath, thereby preparing the access sheath for a delivery device and/or therapy device. The introducer can include an expansion feature that has a larger diameter and/or dimension than an inner diameter/dimension of the access sheath, in at least some configurations of the expansion feature. The introducer can be removed from the access sheath and a delivery device and/or medical device can be inserted through the access sheath with minimal push force. As such, the introducer can be used to reduce, minimize, or prevent harm to the patient and/or avoid damage to the access sheath, delivery device and/or medical device.

In some instances, the present disclosure describes devices, systems, and methods relating to expandable access sheaths comprising an expandable distal end portion configured to be in an open state and a closed state. In some instances, a medical access system can comprise a cord having at least a portion configured to be disposed around a circumferential portion of an expandable distal end portion of an expandable access sheath, while the expandable distal end portion is in the closed state. Maintaining tension on the cord can facilitate maintaining the expandable distal end portion in the closed state. In some instances, an expandable access sheath can comprise an expandable distal end portion comprising a plurality of pairs of pleat portions, where pleat portions of each pair can be configured to be attracted to one another to maintain the expandable distal end portion in the closed state. In some instances, an expandable access sheath can comprise an expandable distal end portion comprising a circumferential portion with a weakened joint. The circumferential portion can break and/or separate along the weakened joint to allow transformation of the expandable distal end portion to the open state. In some instances, an expandable access sheath can have a shaft with an expandable distal end portion comprising one or more weakened portions formed in a sidewall such that the expandable distal end portion can separate along the weakened portions to transform from a closed state to an open state. A distal end of the weakened portion can be spaced from a distal end of the shaft. At least a portion of the weakened portion can have an orientation at an angle relative to the distal end of the shaft, including being perpendicular or substantially perpendicular to the distal end of the shaft. In some instances, the weakened portion can extend along a portion of the expandable distal end portion and be parallel or substantially parallel to a longitudinal axis of the shaft. In some instances, the one or more expandable access sheaths described herein can be used in combination with one or more introducers as described herein.

Methods and structures disclosed herein for treating a patient also encompass analogous methods and structures performed on or placed on a simulated patient, which is useful, for example, for training; for demonstration; for procedure and/or device development; and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof; synthetic; or any combination of natural and synthetic. Virtual elements can be entirely in silica, or overlaid on one or more of the physical components. Virtual elements can be presented on any combination of screens, headsets, holographically, projected, loud speakers, headphones, pressure transducers, temperature transducers, or using any combination of suitable technologies.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

The term “associated with” is used herein according to its broad and ordinary meaning. For example, where a first feature, element, component, device, or member is described as being “associated with” a second feature, element, component, device, or member, such description should be understood as indicating that the first feature, element, component, device, or member is physically coupled, attached, or connected to, integrated with, embedded at least partially within, or otherwise physically related to the second feature, element, component, device, or member, whether directly or indirectly.

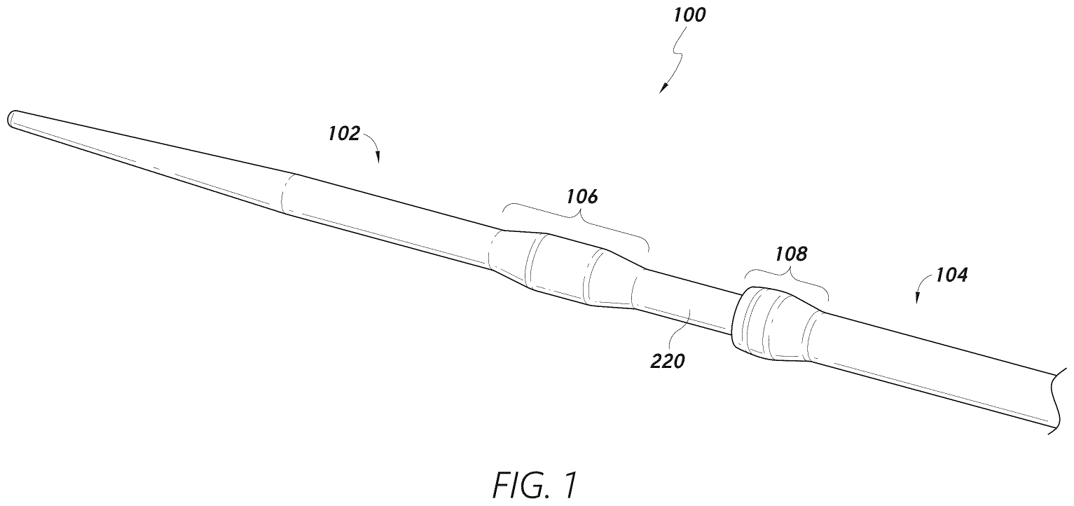

FIG. 1 illustrates an example introducer/access system/assembly 100 according to one or more implementations. The system 100 includes an introducer/dilator 102 and an access sheath 104 configured to receive the introducer 102 within a lumen in the access sheath 104. The introducer 102 (also referred to as “the expansion introducer 102”) can include an elongate shaft/member 220 and an expansion/dilation feature 106 disposed on the elongate shaft/member 220 and configured to radially expand and/or stretch at least a portion of the access sheath 104 when axially moved through the access sheath 104. For example, the introducer 102 can be distally and/or proximally translated through the access sheath 104 such that the expansion feature 106 travels through the lumen of the sheath 104 to radially expand an inner diameter and/or dimension of the access sheath 104. The outer diameter and/or dimension of the expansion feature 106 can be larger than an inner diameter and/or dimension of at least a portion of the access sheath 104. For example, the access sheath 104 can be an expandable access sheath. The outer diameter and/or dimension of the expansion feature 106 can be larger than an inner diameter and/or dimension of at least a portion of the access sheath 104 while the access sheath 104 is in a non-expanded, pre-dilated and/or non-stretched state, such as while a distal end of the sheath 104, a distal portion of the sheath 104, and/or a middle portion of the sheath 104 are in the non-expanded, predilated, and/or non-stretched state. The inner diameter and/or dimension of the access sheath 104 can be a diameter and/or dimension of a lumen extending through at least a portion of the access sheath 104, including through an elongate shaft of the access sheath 104. The outer diameter and/or dimension of the expansion feature 106 can be an outer diameter and/or dimension at a widest portion of the expansion feature 106. In the illustrated example, a portion 108 of the access sheath 104 has been expanded using the introducer 102, wherein the portion 108 maintains an expanded state.

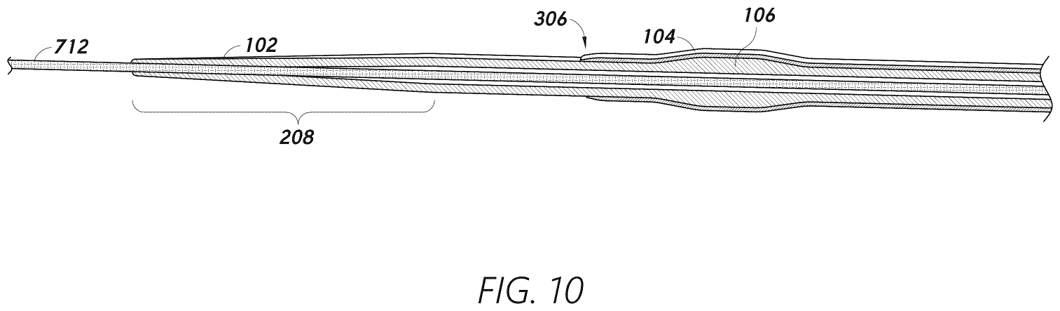

In some cases, the introducer 102 is used in cooperation with the access sheath 104 to advance and/or insert the access sheath 104 into the desired anatomy. In some instances, the introducer 102 can be received by and/or preloaded within the access sheath 104, including while the access sheath 104 is advanced and/or inserted into the desired anatomy. For example, the introducer 102 and the access sheath 104 can be arranged in the configuration shown in FIG. 10, wherein such joined devices can be inserted into the patient. In other cases, an additional introducer is used in cooperation with the access sheath 104 to advance and/or insert the access sheath 104 into the desired anatomy. Here, the additional introducer can be withdrawn from the access sheath 104 after the access sheath 104 is positioned appropriately. The expansion introducer 102 can then be used to stretch and/or dilate the access sheath 104. In either case, the access sheath 104 can generally be inserted along with an introducer disposed therein to provide an atraumatic edge for the access sheath 104 to be inserted into the patient anatomy, thereby providing a smooth advancement of the access sheath 104 into the desired anatomy while reducing, minimizing, or preventing damage to the patient and/or the access sheath 104.

In examples, the introducer 102 and the access sheath 104 can be sized such that the devices, such as the introducer 102 and the access sheath 104, form a relatively tight fit to each other, which can provide support to each other and/or an atraumatic structure. For example, the inner diameter of the access sheath 104 and an outer diameter of the introducer 102 can be dimensioned to be relatively close or the same (e.g., within a threshold amount to each other). In a few non-limiting illustrative examples, an inner diameter of an unexpanded access sheath 104 can be about 6 French (F), about 8 French (F), about 10 French (F), about 12 French (F), or about 14 French (F), while an outer diameter of the introducer 102 can be about 6 French (F), about 8 French (F), about 10 French (F), 12 French (F), or about 14 French (F) and an outer diameter of the expansion feature 106 can be about 10 French (F), about 12 French (F), about 14 French (F) about 16 French (F), about 18 French (F), about 20 French (F), or about 22 French (F). However, any dimensions can be used. In some instances, the access sheath 104 can be sized with the smallest inner diameter and/or the most rigid structure at the distal end of the access sheath 104. The relatively tight fit of the introducer 102 and the access sheath 104 can assist in providing the access sheath 104 with additional structure and/or support when the access sheath 104 is advanced into the desired anatomy. Further, this fit can provide an atraumatic tip for the access sheath 104 (e.g., at the transition of the introducer 102 to the access sheath 104, such as when the introducer 102 is received by and/or preloaded within the access sheath 104, including when the introducer 102 and the access sheath 104 are coupled together).

The access sheath 104 can be elastically and/or temporarily deformable and/or expandable or permanently deformable and/or expandable. In examples, an elastically and/or temporarily deformable and/or expandable access sheath can refer to an access sheath that seeks to maintain an initial inner diameter and/or dimension when an external force is removed. Whereas a permanently deformable and/or expandable access sheath can refer to an access sheath that seeks to maintain an expanded inner diameter and/or dimension until an external force is applied to the access sheath. In some cases, the access sheath 104 includes one or more permanently expandable portions and one or more temporarily and/or elastically expandable portions. FIG. 1 illustrates an example where an inner diameter of the access sheath 104 is permanently expanded at just the distal end portion of the access sheath 104, while the rest of the access sheath 104 is temporarily expanded to allow passage of the introducer 102. However, the access sheath 104 can be entirely of the same type (permanently expandable or temporarily expandable). In alternative instances, the distal end of the access sheath 104 or other portions of the access sheath 104 can be configured to have different characteristics than those illustrated in FIG. 1.

In examples herein, the introducer 102 is discussed in the context of expanding and/or stretching the access sheath 104. Such process can generally include changing one or more properties and/or characteristics of the access sheath 104. For example, when the access sheath 104 is implemented as a permanently expandable sheath, translation of the introducer 102 through the access sheath 104 can change the access sheath 104 from a collapsed and/or non-expanded state having a first inner diameter to an expanded state having a second larger inner diameter. Alternatively, when the access sheath 104 is implemented as a temporarily expandable sheath, translation of the introducer 102 through the access sheath 104 can cause an elasticity and/or stiffness characteristic and/or property of the access sheath 104 to change, such that the access sheath 104 is more malleable and/or deformable after the translation. This can occur in addition to the temporary expansion of a particular section of the access sheath 104 as the introducer 102 passes through the access sheath 104. For example, respective portions of the access sheath 104 can become more malleable and/or deformable after the introducer 102 is advanced through the lumen adjacent to the respective portions. In any case, the introducer 102 can be used to temporarily or permanently expand and/or stretch the access sheath 104, thereby preparing the access sheath 104 for advancement of a medical instrument. For example, a stretched and/or expanded access sheath can allow a medical instrument (which may have a relatively fragile structure) to be pushed and/or advanced through the access sheath and/or beyond the distal end of the access sheath with a relatively small push force, reducing, minimizing, or preventing harm to the patient, medical instrument, and/or access sheath.

Although various examples are illustrated and/or discussed herein in the context of cylindrical introducers and/or sheaths, the introducers and/or sheaths can be implemented in other forms.

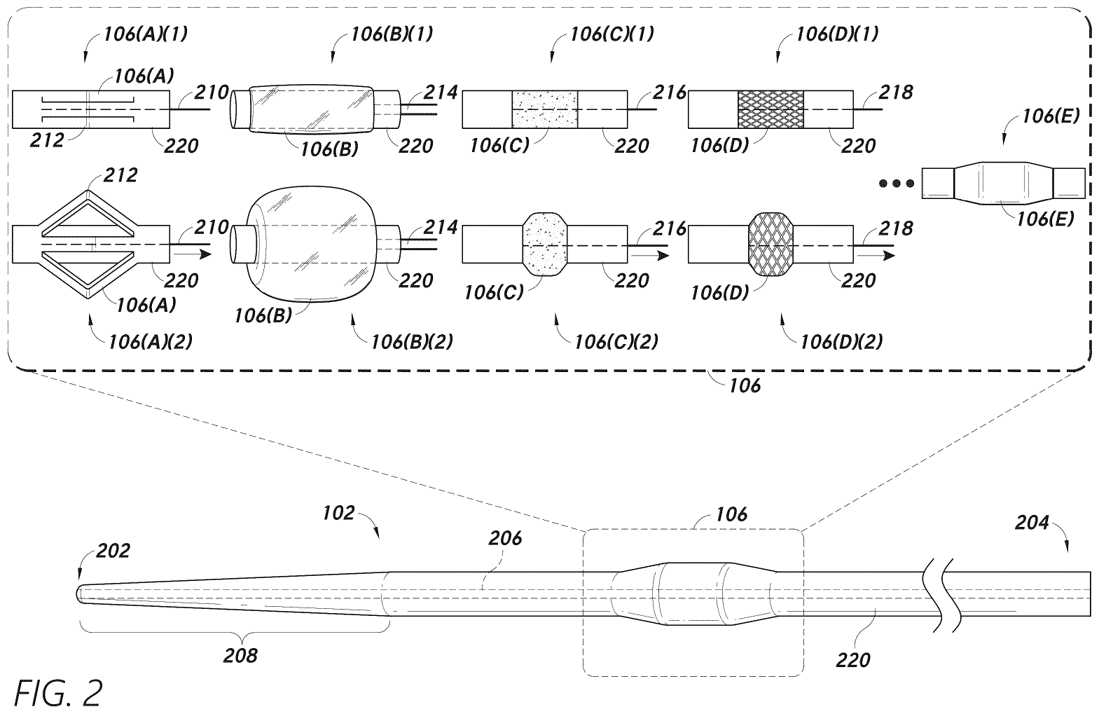

FIG. 2 illustrates example details of the introducer 102 from FIG. 1 according to one or more implementations. As shown in this side view, the introducer 102 includes an elongate shaft 220 having a distal end 202 and a proximal end 204 with the expansion feature 106 disposed between the distal end 202 and the proximal end 204. The expansion feature 106 can include or be actuated to include a larger outer diameter and/or dimension than the outer diameter and/or dimension of at least a neighboring portion of the introducer 102. In examples, such as that illustrated in FIG. 2, the introducer 102 includes a lumen 206 that extends through the introducer 102, such as from the distal end 202 to the proximal end 204, wherein the lumen 206 can be configured to slidably receive a guidewire or other device. For example, the lumen 206 can extend through the elongate shaft/member 220 of the introducer 102. However, the introducer 102 can be implemented without the lumen 206, in some cases. The introducer 102 can include a tapered portion 208 disposed between the distal end 202 and the expansion feature 106. The tapered portion 208 can taper towards the distal end 202 of the introducer 102 (e.g., the distal end 202 can include a smaller outer diameter than the outer diameter of a proximal end of the tapered portion 208). Although the tapered portion 208 is illustrated with a particular length and ends at a particular distance from the expansion feature 106, the tapered portion 208 can include any length. In some alternative instances, a tapered portion can extend from the distal end 202 to or past the expansion feature 106. In some cases, the introducer 102 and an access sheath (not shown in FIG. 2) are joined for advancement into a patient with the tapered portion 208 at least partially extending beyond the distal end of the access sheath. In some instances, the introducer 102 can be preloaded within the access sheath for advancement into the patient with the tapered portion 208 at least partially extending beyond the distal end of the access sheath, for example a relative position of the introducer 102 and the access sheath can be fixed during advancement into the patient.

The expansion feature 106 can allow an access sheath (not shown) to be expanded in phases. For example, if the introducer 102 is distally translated through an access sheath, the expansion feature 106 can incrementally expand the access sheath as the introducer 102 moves through the access sheath. By incrementally expanding and/or stretching the access sheath, the push and/or pull force that is needed to move the introducer 102 can be reduced (in comparison to other solutions), since the friction is greatest at the expansion feature 106, which may be short relative to the length of the access sheath 104 and/or introducer 102. In some cases, one or more dimensions of the expansion feature 106 can be designed to provide the desired push and/or pull force on the introducer 102 when axially moving the introducer 102 through access sheath. For example, an axial length of the expansion feature 106, an outer diameter of the expansion feature 106, a length of tapering at the beginning or end of the expansion feature 106, and/or other dimensions of the expansion feature 106 can be configured to satisfy the appropriate requirements.

The expansion feature 106 can be implemented in a variety of manners. Referring again to FIG. 2, in one example, the expansion feature 106 is implemented with one or more ribs/bendable strips 106(A) that are configured to radially expand when a force is exerted on the ribs/bendable strips 106(A) to decrease/shrink a length of the ribs/bendable strips 106(A). In some instances, the one or more ribs/bendable strips 106(A) can form a portion of the elongate shaft 220, for example being coupled to or integrated as a part of the elongate shaft 220. For instance, a pull wire/elongate movement member 210 can be attached to a distal side of the ribs/bendable strips 106(A), or distal end of the introducer 102, and extend through the elongate shaft 220 of the introducer 102, such as through the lumen 206 or a dedicated lumen for the pull wire 210. In some instances, the pull wire/elongate movement member 210 can be attached to a portion of the elongate shaft 220 distal of the ribs/bendable strips 106(A). The pull wire 210 can be actuated and/or pulled proximally to radially expand the ribs/bendable strips 106(A). In particular, the ribs/bendable strips 106(A) can be compressed using the pull wire 210 to cause the ribs/bendable strips 106(A) to bend and/or buckle at a point/portion 212, which can include an indentation, more deformable material, hinge, and/or other structure and/or form. The ribs/bendable strips 106(A) are illustrated in a non-expanded form at 106(A)(1) and illustrated in an expanded form at 106(A)(2). In a non-expanded form, the ribs/bendable strips 106(A) can include substantially the same outer diameter as the rest of the shaft of the introducer 102.

In another example, the expansion feature 106 can be implemented with a balloon 106(B) attached to the elongate shaft of the introducer 102. In some instances, the balloon 106(B) can be disposed around the elongate shaft 220 of the introducer 102. The balloon 106(B) can be inflated and/or deflated with a fluid and/or gas via a tube/channel 214 that is connected to the balloon 106(B). For example, the tube/channel 214 can be in fluid communication with the balloon 106(B) such that fluid and/or gas can be delivered to or withdrawn from the balloon 106(B) for inflating or deflating the balloon 106(B), respectively. The balloon 106(B) can be implemented with a variety of materials to facilitate expansion and/or deflation. The balloon 106(B) is illustrated in a non-expanded form at 106(B)(1) and illustrated in an expanded form at 106(B)(2). In a non-expanded form, the balloon 106(B) can be relatively tight to the elongate shaft of the introducer 102 such that the balloon 106(B) has the same or substantially the same outer diameter as the elongate shaft. For example, the balloon 106(B) can assume a low profile while in the non-expanded form such that an outer diameter of the balloon 106(B) can be the same or similar to that of the elongate shaft 220.

In a further example, the expansion feature 106 can be implemented with a section 106(C) that includes a different type of material than the rest of and/or neighboring sections of, the elongate shaft of the introducer 102. For instance, the section 106(C) can be formed of a material that is more deformable, malleable, and/or compressible than an adjacent section of the introducer 102, including an adjacent portion of the elongate shaft 220. The section 106(C) can be formed of a softer material than an adjacent section. A pull wire/elongate movement member 216 can be coupled to a portion of the introducer 102, including a portion of the expansion feature 106, or a portion of the introducer 102 distal of the expansion feature 106, including a portion of the elongate shaft 220 distal of the section 106(C). In some instances, the pull wire/elongate movement member 216 can be attached to a distal side of the section 106(C). In some instances, the pull wire/elongate movement member 216 can be coupled to a portion of the introducer 102 distal of the section 106(C), including a distal end of the introducer 102. The pull wire/elongate movement member 216 can extend through the elongate shaft of the introducer 102, such as through the lumen 206 or a dedicated lumen. The pull wire 216 can be actuated/pulled proximally to deform the section 106(C) in a radially direction (e.g., expanding the section 106(C)). Although a different type of material is discussed in the context of the section 106(C), any of the implementations 106(A)-106(E) can include a different type of material than other portions of the introducer 102.

In yet another example, the expansion feature 106 is implemented with a braided/stent/mesh section 106(D) that is configured to radially expand when a force is exerted on the braided section 106(D). For instance, a pull wire/elongate movement member 218 can be coupled to a portion of the introducer 102, including a portion of the expansion feature 106, or a portion of the introducer 102 distal of the expansion feature 106. In some instances, the pull wire/elongate movement member 218 can be attached to a portion of the elongate shaft 220 distal of the braided section 106(D). In some instances, the pull wire/elongate movement member 216 can be attached to a distal side of the braided section 106(D). In some instances, the pull wire/elongate movement member 218 can be coupled to a portion of the introducer 102 distal of the braided section 106(D), including a or distal end of the introducer 102. The pull wire/elongate movement member 218 can extend through the elongate shaft of the introducer 102, such as through the lumen 206 or a dedicated lumen. The pull wire 218 can be actuated and/or pulled proximally to radially expand the braided section 106(D). In particular, the braided section 106(D) can be compressed using the pull wire 218 (e.g., in a similar manner as that discussed above for the ribs 106(A)) to cause the braided section 106(D) to enlarge the outer diameter and/or dimension of the braided section 106(D). The braided section 106(D) is illustrated in a non-expanded form at 106(D)(1) and illustrated in an expanded form at 106(D)(2). In a non-expanded form, the braided section 106(D) can include the same or substantially the same outer diameter as adjacent portions of the elongate shaft of the introducer 102. In some instances, the braided section 106(D), while in the non-expanded form, can include the same or substantially the same outer diameter as the rest of the elongate shaft of the introducer 102. The braided section 106(D) can be implemented with a mesh-like form using a variety of materials, such as a polymer, a metal, etc.

Although various examples are discussed in the context of using a pull wire to enable and/or actuate an expansion feature, a rigid elongate shaft can additionally, or alternatively, be implemented, such as by pushing and/or pulling the rigid elongate shaft to deform/form the expansion feature.

In an additional example, the expansion feature 106 can be implemented as a fixed feature 106(E) (sometimes referred to as “the fixed-form expansion feature 106(E)”) along the elongate shaft 220 of the introducer 102. For instance, the fixed expansion feature 106(E) can be formed by extruding a material onto or along with the manufacturing of the elongate shaft 220 of the introducer 102 (e.g., through a reflow or melt process), molding the elongate shaft 220 with the feature 106(E), attaching and/or fastening one or more elements to the elongate shaft 220 (e.g., using an adhesive, mechanical fastener, soldering material, or another fastening mechanism to attach a larger diameter shaft to the elongate shaft 220 of the introducer 102), or otherwise forming a fixed feature on the elongate shaft 220. The fixed expansion feature 106(E) can be a separate component from the elongate shaft 220 of the introducer 102 or integral with the elongate shaft 220. The fixed expansion feature 106(E) includes a first tapered section at a distal portion and a second tapered section at a proximal portion, wherein the tapered sections provide a smooth transition to the fixed expansion feature 106(E). However, the fixed expansion feature 106(E) can be implemented with a more abrupt transition to the elongate shaft. In some cases, the fixed expansion feature 106(E) is referred to as “a radially-protruding bump.”

The implementations 106(A)-106(D) (and other implementations discussed herein) can be referred to as active/articulable expansion features that can be selectively enabled/disabled, such as by actuating a pull wire, providing fluid and/or gas, etc. As such, these implementations can be independently controlled (e.g., separate from the movement of the introducer 102). An active expansion feature can be enabled/radially expanded while the expansion feature is positioned at a particular location, such as within a particular portion of an access sheath, extended out a distal end of an access sheath, etc. In contrast, the implementation 106(E) (and other implementations discussed herein) can be referred to as non-active, non-articulable, and/or fixed expansion feature that is generally fixed in the same form (e.g., is not activated by some form of manipulation and/or actuation).

The introducer 102 can include a variety of materials, such as a polymer, a metal, etc. In some examples, the expansion feature 106 includes a different type of material than the rest of the introducer 102. While in other examples, the expansion feature is implemented with the same type of material.

Although the introducer 102 is illustrated with a single expansion feature, the introducer 102 can include multiple expansion features. For example, the introducer 102 can include a first expansion feature positioned a distance from the distal end 202 and a second expansion feature positioned farther from the distal end 202. The first expansion feature can include a first outer diameter and/or dimension and the second expansion feature can include a second outer diameter and/or dimension, wherein the first outer diameter and/or dimension is smaller (or larger than) the second outer diameter and/or dimension. This can allow an access sheath to be expanded in phases.

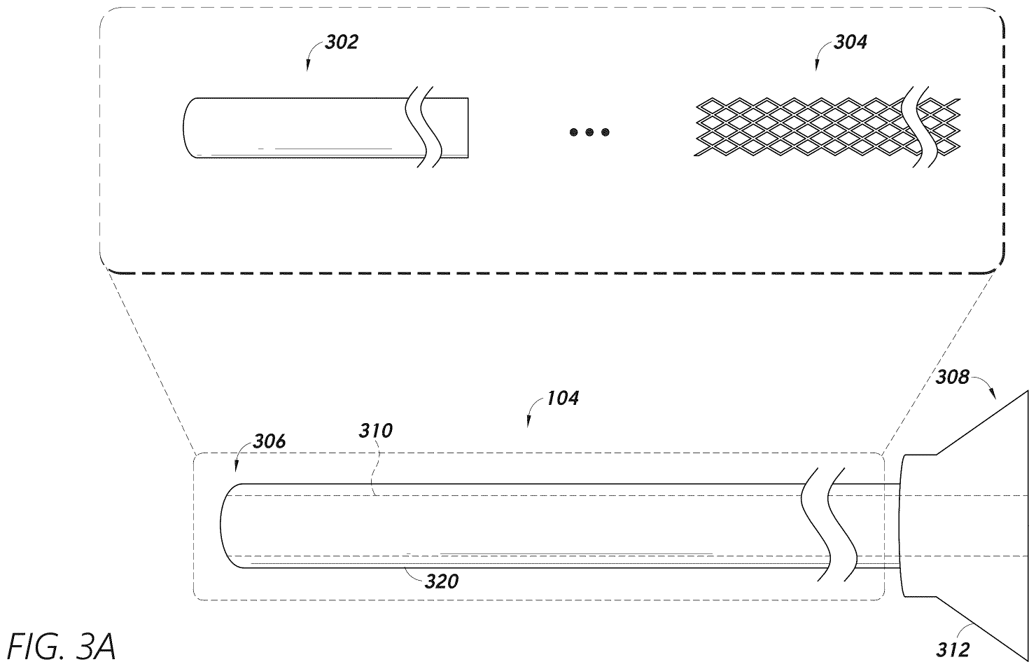

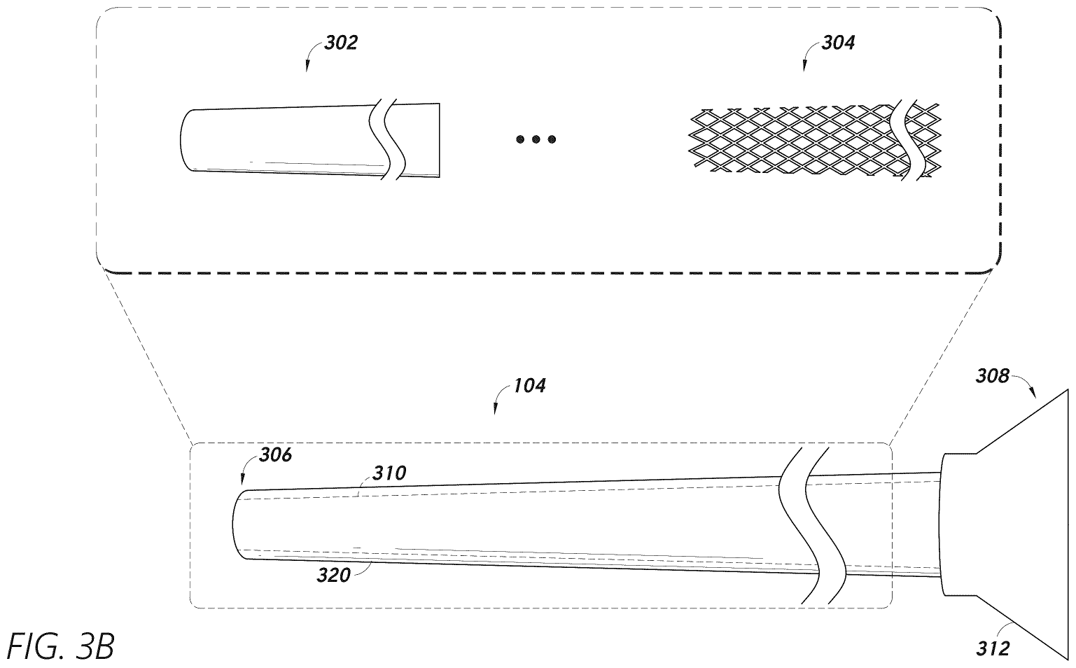

FIGS. 3A and 3B illustrate example implementations of the access sheath 104 from FIG. 1 according to one or more implementations. FIG. 3A illustrates the access sheath 104 having a straight or substantially straight shape and/or form, while FIG. 3B illustrates the access sheath 104 having a tapered shape and/or form. For example, an elongate shaft 320 of the access sheath 104 in FIG. 3A can have a straight or substantially straight shape and/or form. An elongate shaft 320 of the access sheath 104 in FIG. 3B can have a tapered shape and/or form. In the example side views of FIG. 3A and/or FIGS. 3B, at least a portion of the access sheath 104, including the elongate shaft 320 of the access sheath 104, can be expandable and/or stretched, as discussed above. This can allow the access sheath to be advanced into the patient (at least the distal end of the access sheath 104, such as a distal end of the elongate shaft 320) with a relatively small profile and/or diameter, and then expanded to a larger diameter to facilitate delivery of a medical device through the access sheath 104.

The access sheath 104 can be implemented in a variety of forms. For example, as shown at 302 in FIGS. 3A and 3B, at least a portion of the access sheath 104 can be solid or substantially solid, at least regarding an outer wall of the access sheath 104, which has a lumen 310 therein to facilitate access for another device. For example, the elongate shaft 320 can comprise at least a solid portion or substantially solid portion adjacent to and/or defining a corresponding portion of the lumen 310. Alternatively or in combination, as shown at 304 in FIGS. 3A and 3B, the access sheath 104 can include a mesh and/or stent form, which can be more easily expanded in comparison to a solid form depending on the types of materials used to form the access sheath 104, dimensions of the access sheath 104 and/or other characteristics/properties. For example, the elongate shaft 320 can comprise at least a mesh and/or stent form adjacent to and/or defining a corresponding portion of the lumen 310.

In the examples of FIGS. 3A and 3B, the access sheath 104 includes a distal end 306 that is configured to be inserted into the patient and the proximal end 308 that is configured to receive the introducer 102 and/or another device for entry into the access sheath 104. The access sheath 104 can include the elongate shaft/member 320 that has a lumen 310 extending from the distal end 306 to the proximal end 308. Here, the proximal end 308 includes a port 312 configured to receive the introducer 102, another device and/or connect to various medical instruments for facilitating a procedure. Although the port 312 is illustrated, other components can alternatively, or additionally, be attached to the proximal end 308 of the access sheath 104. In FIG. 3B, the lumen 310 is tapered along with the outer diameter of the access sheath 104, such as with the outer diameter of the elongate shaft/member 320 of the access sheath 104; however, the lumen 310 can maintain the same or substantially the same inner diameter, in some cases.

In examples, the tapered configuration of the access sheath 104, such as the elongate shaft 320, shown in FIG. 3B can allow the access sheath 104 to be advanced into a patient in a manner that dilates the anatomy of the patient incrementally. In particular, the distal end 306 can include a smaller outer diameter than the proximal end 308, a central portion or mid-portion of the access sheath, and/or another portion located more proximally than the distal end 306. The distal end 306 can include a smaller outer diameter than a mid-portion of the elongate shaft 320, and/or another portion of the elongate shaft 320 located more proximally than the distal end 306. This can reduce, minimize, or prevent damage to the anatomy of the patient and/or prepare the anatomy for additional devices that are advanced into the patient.

In some examples where the access sheath 104 is tapered, an introducer (e.g., an expansion introducer or non-expansion introducer) can be implemented with a tapered form to generally fit and/or match to the access sheath 104 when the introducer is positioned at a particular position relative to the access sheath 104, such as with a particular amount of the introducer extending beyond the distal end of the access sheath 104.

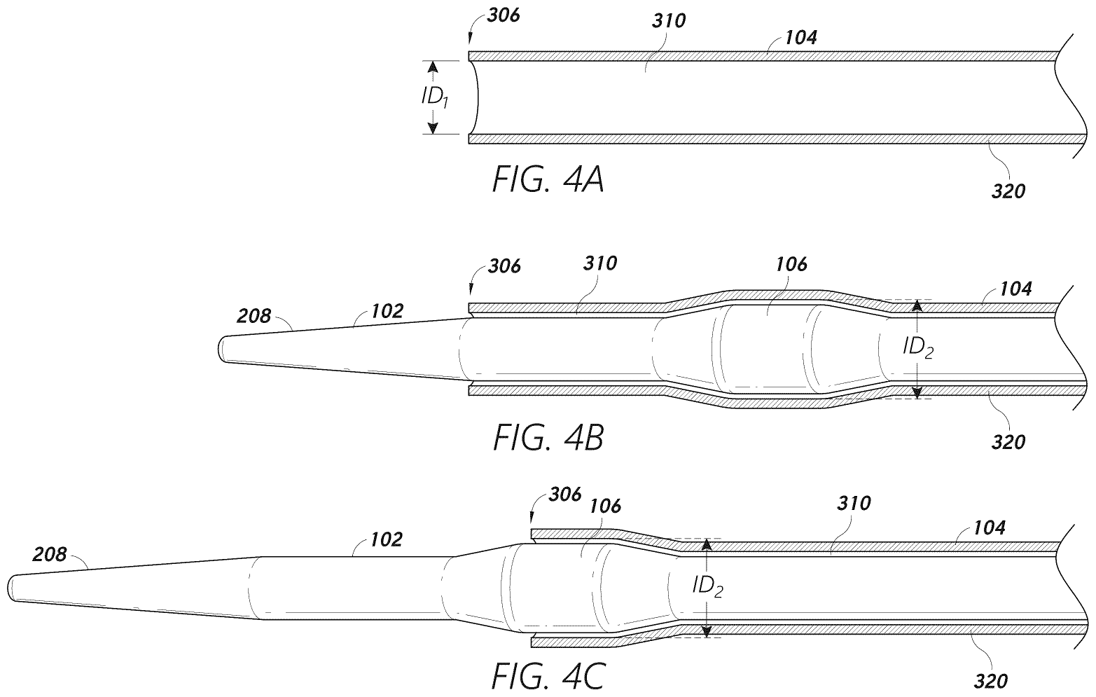

FIGS. 4A through 4C illustrate an example of the access sheath 104 implemented as an elastically and/or temporarily expandable sheath. That is, the access sheath 104 can be temporarily and/or elastically expanded while the expansion feature 106 of the introducer 102 is advanced through the access sheath 104. For example, as shown in FIG. 4A, the access sheath 104, such as the elongate shaft 320 of the access sheath 104, can have an initial inner diameter ID₁. As the introducer 102 is advanced through the access sheath 104, the inner diameter of the access sheath 104, such as the elongate shaft 320 of the access sheath 104, can temporarily expand to a larger inner diameter ID₂, as illustrated in FIGS. 4B and 4C. For example, a widest portion of the expansion feature 106 can have a diameter of ID₂. In some instances, respective portions of the access sheath 104, such as the elongate shaft 320, can temporarily and/or elastically expand to the larger inner diameter ID₂ as the expansion feature 106 is advanced through corresponding portions of the lumen 310. In one example, the introducer 102 can be advanced to the position shown in FIG. 4B to insert the access sheath 104 and the introducer 102 into the patient's anatomy. A portion of the introducer 102 can be disposed distally of the access sheath 104, including for example the tapered portion 208. In some instances, the tapered portion 208 can be disposed distally of the access sheath 104, while portions of the introducer 102 proximal of the tapered portion 208 can be proximal of the distal end 306 of the access sheath 104. This can provide an atraumatic edge/tip for inserting the access sheath 104. Portions of the introducer 102 can then be advanced through the distal end 306 of the access sheath 104 (as shown in FIG. 4C) after the access sheath 104 is positioned in the desired anatomy. For example, the introducer 102 can be distally advanced relative to the access sheath 104 to position more of the introducer 102 distally of the distal end 306.

In this example, the inner diameter (ID) of the access sheath 104 may return to the same or substantially the same dimension as the initial inner diameter ID₁ of the access sheath 104 (shown in FIG. 4A) after the introducer 102 has traveled through the access sheath 104. For example, the inner diameter (ID) of the access sheath 104 may return to the same or substantially the same dimension as the initial inner diameter ID₁ of the access sheath 104 (shown in FIG. 4A) after the introducer 102 is withdrawn from the lumen 310. In some instances, the access sheath 104 can be stretched and/or reconfigured and/or the inner diameter of the distal end 306 can be expanded (in cases where the inner diameter is smaller at the distal end 306). For example, the translation of the introducer 102 through the access sheath 104 can cause an elasticity and/or stiffness characteristic and/or property of the access sheath 104 to change, such that the access sheath 104 is more malleable and/or deformable. For example, the elongate shaft 320 can be more malleable and/or deformable after the elongate shaft 320 has been stretched by the introducer 102. This can reduce or minimize a push force needed to advance another device through the lumen 310 and/or out the distal end 306 of the access sheath 104. In some cases where the access sheath 104 is temporarily and/or elastically expandable, the access sheath 104 may be less traumatic to the patient's anatomy, since the sheath 104 is expanded momentarily.

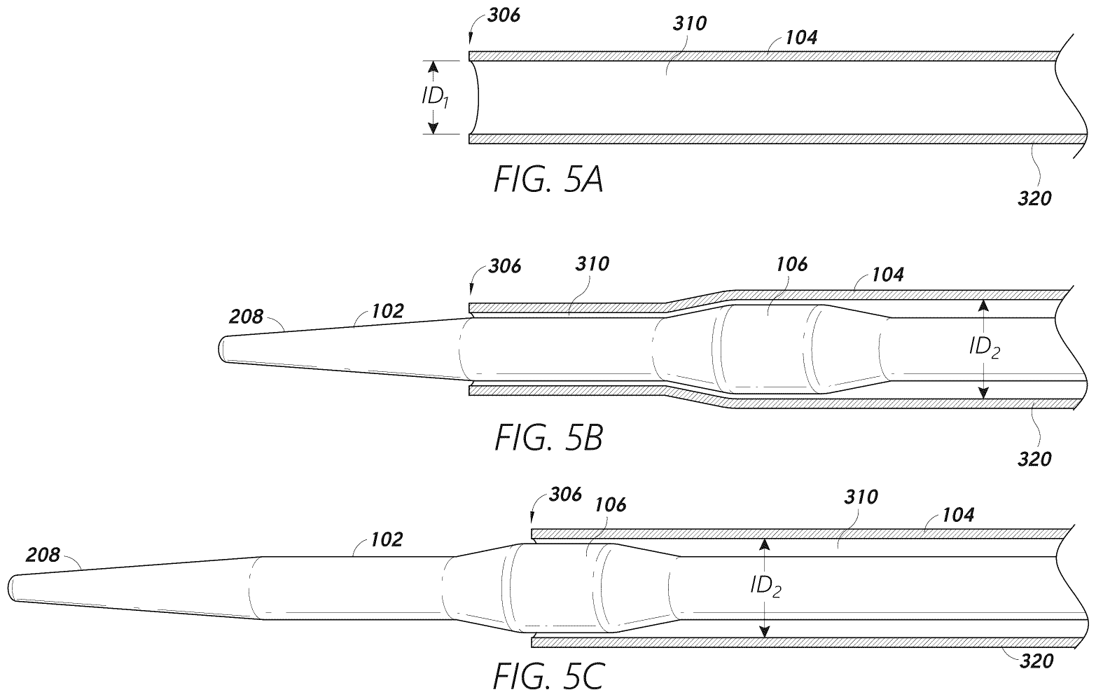

FIGS. 5A through 5C illustrates an example of the access sheath 104 implemented as a permanently expandable sheath according to one or more implementations. That is, the access sheath 104 can be permanently expanded due to advancement of the expansion feature 106 through the access sheath 104. For example, the elongate shaft 320 of the access sheath 104 can be permanently expanded after advancement of the expansion feature 106 through the lumen 310 of the access sheath 104. As similarly discussed above in reference to FIG. 4A, the access sheath 104 can have an initial inner diameter ID₁, as shown in FIG. 5A. As the introducer 102 is advanced through the access sheath 104, the sheath 104 can permanently expand to a larger inner diameter ID₂, as illustrated in FIGS. 5B and 5C. For example, a widest portion of the expansion feature 106 can have a diameter of ID₂. In some instances, respective portions of the access sheath 104, such as the elongate shaft 320, can permanently expand to the larger inner diameter ID₂as the expansion feature 106 is advanced through corresponding portions of the lumen 310. As noted above, a permanently expandable sheath can refer to a sheath that seeks to maintain an expanded diameter until an external force is applied. In some instances, the elongate shaft 320 can maintain or substantially maintain the larger inner diameter ID₂. In some instances, the elongate shaft 320 can maintain or substantially maintain the larger inner diameter ID₂ after the introducer 102 is withdrawn from the lumen 310.

Referring to FIG. 5B, the introducer 102 can be preloaded within the access sheath 104 during advancement of the access sheath 104 to desired position within the patient. A portion of the introducer 102 can be disposed distally of the access sheath 104, including for example the tapered portion 208 to provide an atraumatic edge/tip during insertion of the access sheath 104. For example, the tapered portion 208 can be disposed distally of the access sheath 104, while portions of the introducer 102 proximal of the tapered portion 208 can be proximal of the distal end 306 of the access sheath 104. After the access sheath 104 is desirably positioned, the introducer 102 can then be further translated distally to advance portions of the introducer 102 through the distal end 306 of the access sheath 104, as shown in FIG. 5C.

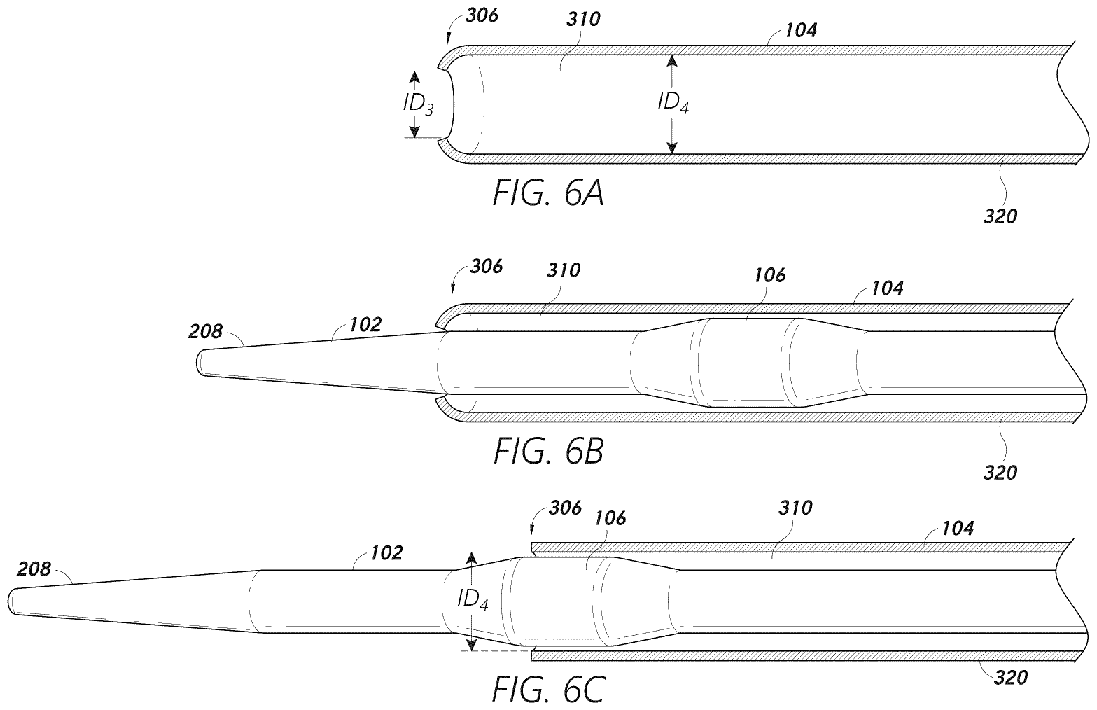

FIGS. 6A through 6C illustrate an example implementation of the access sheath 104 with a smaller and/or crimped inner diameter ID₃ at the distal end 306 of the access sheath 104. This example is discussed in the context of the expansion feature 106 of the introducer 102 having the same or substantially the same outer diameter as an inner diameter of the access sheath 104, except for the inner diameter of the distal end 306. However, the outer diameter of the expansion feature 106 can be larger than the initial inner diameter of the access sheath 104. The access sheath 104 shown in FIGS. 6A through 6C can be implemented as a temporarily or permanently expandable sheath.

In FIG. 6A, most of the access sheath 104 can have an initial inner diameter ID₄. For example, portions of the elongate shaft 320 of the access sheath 104 proximal of the distal end 306 can have the initial inner diameter ID₄. As the introducer 102 is advanced through the access sheath 104, the introducer 102 can expand any portions of the access sheath 104 to match the outer diameter of the expansion feature 106. In some instances, the initial inner diameter ID₄ can be larger than a widest portion of the expansion 106. In some instances, the initial inner diameter ID₄ can be smaller than a widest portion of the expansion 106 such that advancement of the expansion feature 106 through the lumen 310 can cause expansion of respective portion of the access sheath 104, including respective portions of the elongate shaft 320 of the access sheath 104. Here, the distal end 306 of the access sheath 104 includes a smaller and/or crimped inner diameter ID₃. The smaller and/or crimped inner diameter ID₃ can be shorter than the initial inner diameter ID₄. As such, the expansion feature 106 expands and/or breaks open the distal end 306 of the access sheath 104 as the expansion feature 106 passes through the distal end 306, as shown in FIG. 6C. Thus, the inner diameter of the distal end 306 can be expanded, including for example to the inner diameter ID₄ of the rest of the access sheath 104. In some examples, the introducer 102 is used to ensure that an inner diameter of the access sheath 104, including an inner diameter of the elongate shaft 320, is the same or substantially the same, or larger than, a particular dimension, thereby avoiding excessive push forces when inserting a medical device out the distal end 306 of the access sheath 104.

Referring to FIG. 6B, in some instances, the introducer 102 can be preloaded within the access sheath 104 during advancement of the access sheath 104 to desired position within the patient. A portion of the introducer 102 can be disposed distally of the access sheath 104, including for example the tapered portion 208 to provide an atraumatic edge/tip during insertion of the access sheath 104. For example, the tapered portion 208 can be disposed distally of the access sheath 104, while portions of the introducer 102 proximal of the tapered portion 208 can be proximal of the distal end 306 of the access sheath 104. After the access sheath 104 is desirably positioned, the introducer 102 can then be further translated distally to advance portions of the introducer 102 through the distal end 306 of the access sheath 104, as shown in FIG. 6C. Advancing the introducer 102 relative to the access sheath 104 can cause expansion of the inner diameter of the distal end 306, including expansion to the inner diameter ID₄ of the rest of the access sheath 104.

In examples, the distal end 306 of the access sheath 104 can be permanently or temporarily expanded to the inner diameter ID₄ (as shown in FIG. 6C). Alternatively, in examples, the distal end 306 of the access sheath 104 can be permanently or temporarily expanded to an inner diameter larger than ID₄.

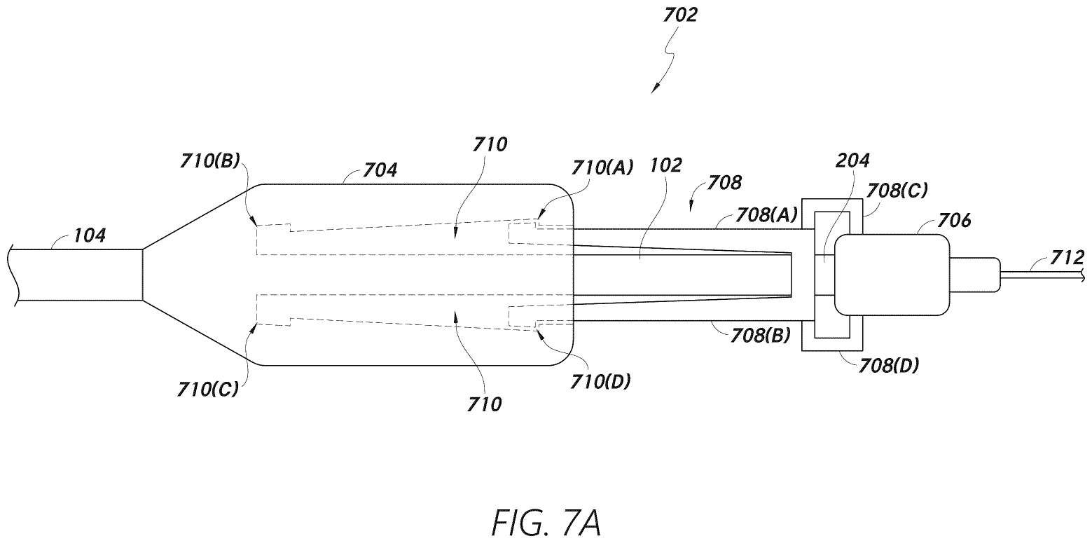

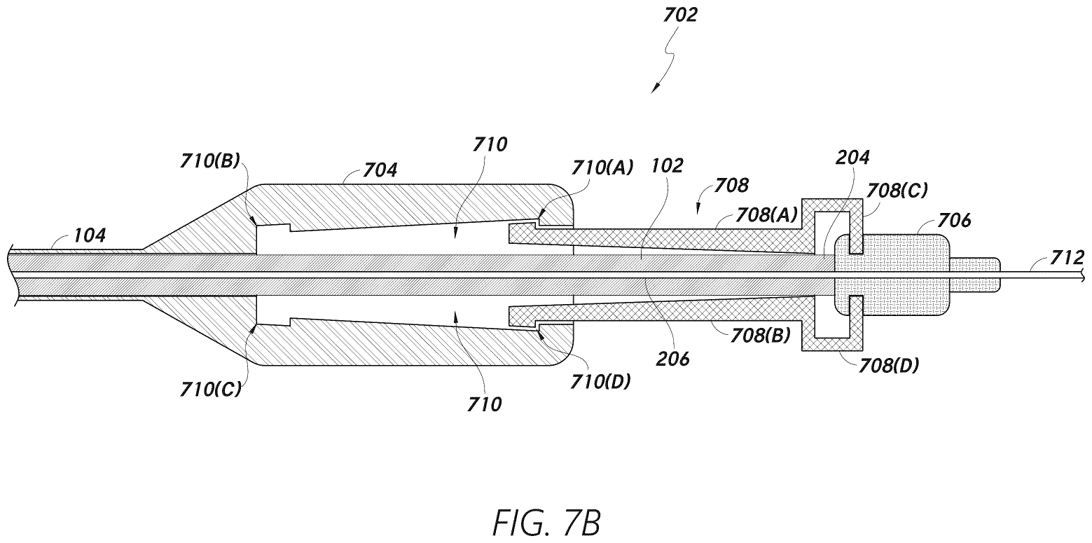

FIGS. 7A and 7B illustrate an example locking mechanism/assembly/means 702 to ensure that the introducer 102 has been axially translated before the introducer 102 is removed from the access sheath 104 according to one or more implementations. FIG. 7A illustrates a side view of the locking mechanism 702, while FIG. 7B illustrates a cross-sectional view of the locking mechanism 702. The locking mechanism 702 can be detachably and/or selectively coupled to the introducer 102 and/or the access sheath 104 to prevent removal of the introducer 102 from the access sheath 104 (e.g., to retain and/or lock the introducer 102) until the introducer 102 is translated distally. For example, the locking mechanism 702 can engage with the introducer 102 and disengage from the introducer 102 when the introducer 102 is translated distally by more than a particular distance such that the expansion feature 106 travels beyond the distal end 306 of the access sheath 104 (not shown in FIGS. 7A and 7B). This can ensure that the distal end 306 and/or other portions of the access sheath 104 are fully expanded and/or stretched before the introducer 102 is removed from the access sheath 104. As such, the locking mechanism 702 can be implemented as a safety feature to ensure the appropriate operation of the introducer 102 and access sheath 104.

As shown in FIGS. 7A and 7B, the locking mechanism 702 includes a sheath hub 704 coupled to or integral with the access sheath 104 and an introducer hub 706 coupled to or integral with the introducer 102. In many cases, the sheath hub 704 and the access sheath 104 do not move relative to each other. Similarly, the introducer hub 706 and the introducer 102 may not generally move relative to each other. Although, in some instances, the hubs 704 and 706 are disposed on the distal ends of the access sheath 104 and introducer 102, respectively, the hub 704 and/or hub 706 can be positioned at other locations.

In this example, the introducer hub 706 can be detachably coupled to a clip/locking member 708, which is configured to move axially within a space(s)/cavity 710 in the sheath hub 704. For example, the clip/locking member 708 can be translated proximally and distally within the space(s)/cavity 710. When the introducer hub 706 is coupled to the clip/locking member 708, the introducer hub 706 and the clip/locking member 708 can move together through the sheath hub 704 based on the axial constraints of the space(s)/cavity 710. For example, an arm 708(A) of the clip/locking member 708 can slide, such as translating proximally and/or distally, within the space(s)/cavity 710 between positions 710(A) and 710(B), while an arm 708(B) can slide, such as translating proximally and/or distally, within the space(s)/cavity 710 between positions 710(D) and 710(C).

The sheath hub 704 can be configured to release and/or unlatch the introducer hub 706 from the clip/locking member 708 when the clip/locking member 708 is positioned at a distal end of the space(s)/cavity 710. For example, the space(s)/cavity 710 can be tapered to pinch and/or move the arms 708(A) and 708(B) radially inward (e.g., closer together) as the clip/locking member 708 is distally moved to the positions 710(B) and 710(C). When the arms 708(A) and 708(B) are positioned at 710(B) and 710(C), respectively, portions/clip members 708(C) and 708(D) of the clip/locking member 708 can release the introducer hub 706 (e.g., open and/or move radially outward to detach from the introducer hub 706). For instance, the arm 708(A) can be actuated and/or leveraged to control motion of the portion 708(C), while the arm 708(B) can be actuated and/or leveraged to control motion of the portion 708(D).

The space(s)/cavity 710 can include stopping features (e.g., lips, ridges, designated cavities, and/or another locking feature) at the positions 710(B) and 710(C) to prevent the arms 708(A) and 708(B) from moving proximally once the stopping features are reached. That is, the arms 708(A) and 708(B) can be held/remain fixed at the positions 710(B) and 710(C) when such positions are reached. This can allow the introducer hub 706 to be released from the clip/locking member 708 and the introducer 102 to be pulled out of the access sheath 104.

In examples, the length of the space(s)/cavity 710 and/or length of the clip/locking member 708 can be correlated to the length of the introducer 102 and/or access sheath 104 and/or a position of the expansion feature 106 on the introducer 102, such that the expansion feature 106 of the introducer 102 extends beyond the distal end 306 of the access sheath 104 when the clip/locking member 708 is positioned at the distal end of the space(s)/cavity 710 (e.g., the positions 710(B) and 710(C)). As such, the introducer hub 706 can disengage from the clip/locking member 708 when the introducer 102 is translated distally within the access sheath 104 by more than a threshold distance. The threshold distance can be associated with and/or correlated to the length of the introducer 102, the length of the access sheath 104, a position of the expansion feature 106 (e.g., relative to the proximal and/or distal end of the introducer 102, relative to the introducer hub 706, etc.), the length of the space(s)/cavity 710, and/or length of the clip/locking member 708.

In the example of FIGS. 7A and 7B, the introducer 102 is configured to receive a guidewire 712 or another device through the lumen 206 of the introducer 102, which can assist in navigating the introducer 102 and/or access sheath 104. However, the introducer 102 can be implemented without a lumen to receive the guidewire 712. The introducer 102 can generally slide along the guidewire 712.

Although a particular structure is illustrated for the locking mechanism 702, the locking mechanism 702 can be implemented in a variety of other manners. For example, the locking mechanism can be implemented as a cinch mechanism, such as a Tuohy Borst, collet, set screw, cam, and/or another element in the sheath hub 704 and/or the introducer hub 706.

FIGS. 8-1, 8-2 and 8-3 illustrate an example flow diagram of a process 800 for dilating and/or expanding an access sheath using an introducer in accordance with one or more implementations of the present disclosure. FIGS. 9-1, 9-2 and 9-3 shows certain images of example anatomy and certain devices associated with the process 800 of FIGS. 8-1, 8-2 and 8-3 to illustrate aspects of the process 800 according to one or more implementations thereof. The process 800 is illustrated as a sequence of blocks. The blocks can be arranged in any order and are not necessarily limited to the particular order illustrated. The acts and/or operations of the blocks can be performed by a physician and/or user, and/or a device and/or component.

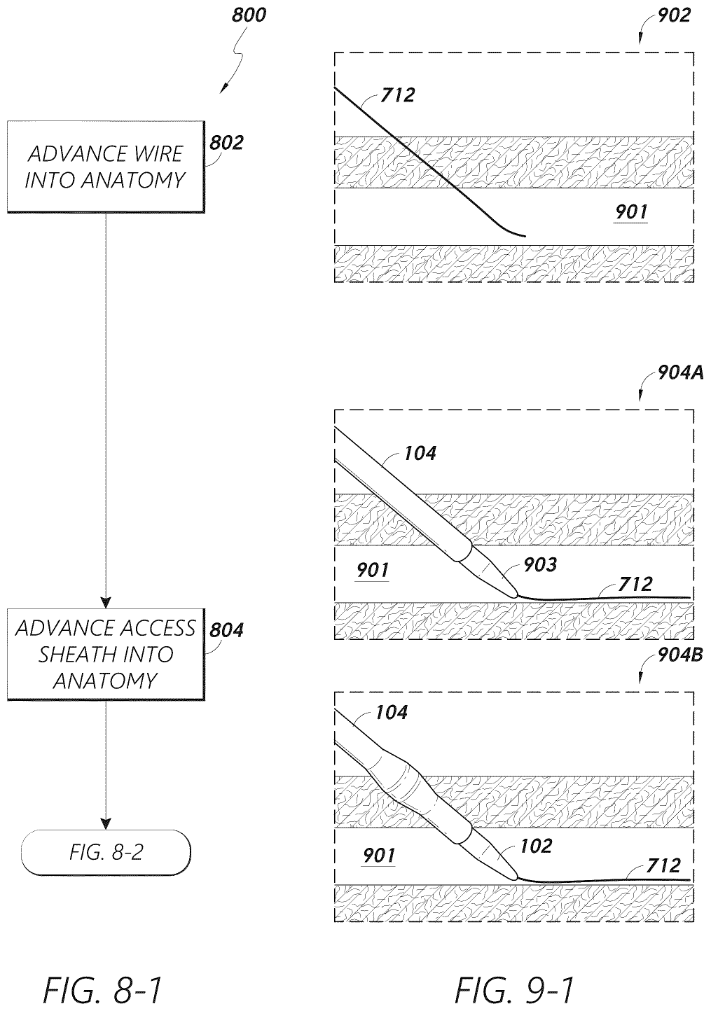

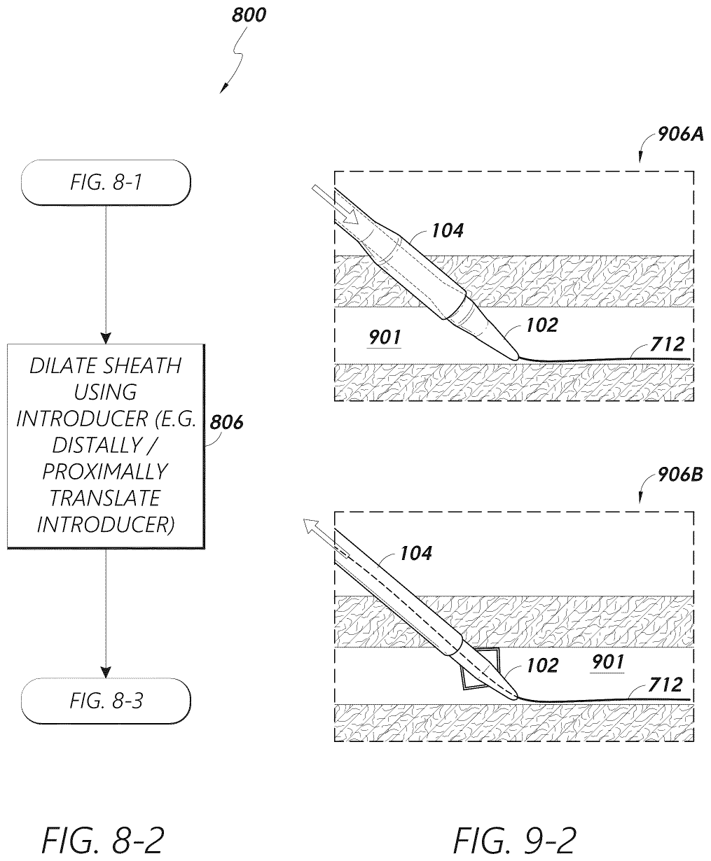

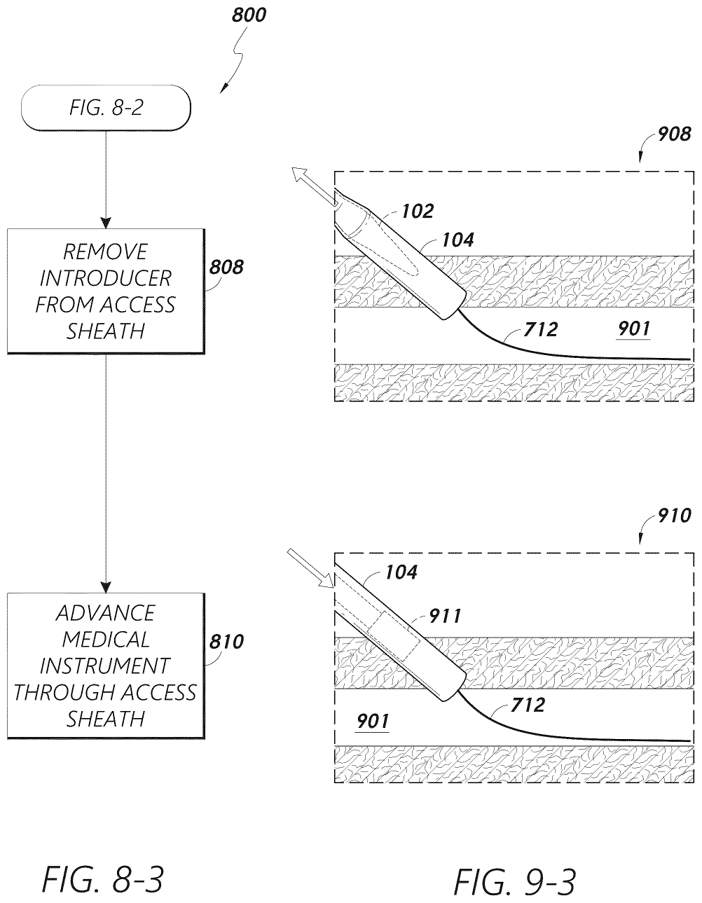

FIG. 8-1 shows blocks 802 and 804 of the process 800. Images 902, 904A and 904B of FIG. 9-1 show images corresponding to the blocks 802 and 804. At block 802, the process 800 includes advancing a wire/guidewire 712 into the desired anatomy. For example, as shown in image 902, the wire/guidewire 712 can be inserted through the skin and/or other tissue into a blood vessel or other anatomy 901. The guidewire/wire 712 can be used to navigate the introducer 102, access sheath 104, medical instrument, and or other devices within the anatomy of the patient, as discussed below. In some cases, a needle or another wire is first used to create a passage for the guidewire 712. However, the wire/guidewire 712 can be advanced without such devices.

At block 804, the process 800 includes advancing an access sheath into the anatomy. For example, as shown in images 904A and 904B, a distal portion of the access sheath 104 can be advanced and/or inserted into the blood vessel 901. The access sheath 104 can be coupled to an introducer 102/903 and advanced into the blood vessel 901 with the introducer 102/903 disposed therein. The introducer 102/903 can provide an atraumatic edge/point for inserting the access sheath 104 into the blood vessel 901. The access sheath 104 can be advanced into the patient such that at least the distal end of the access sheath 104 is located within the blood vessel 901. This can create a port and/or access point into the blood vessel 901 (e.g., for a therapy/delivery device to access the blood vessel 901), as discussed in further detail below.

In one example, as shown in image 904A, the access sheath 104 can be advanced with a non-expansion type introducer 903, which is free from an expansion feature. For example, the introducer 903 can include a straight or tapered form introducer that is generally not configured to expand the access sheath 104. Here, the introducer 903 can be inserted into the access sheath 104 outside the patient, such that the distal end portion of the introducer 903 extends beyond the distal end of the access sheath 104 (in the configuration shown in the image 904A). The combined/coupled introducer 903 and the access sheath 104 can then be inserted into the blood vessel 901. In some cases, the introducer 903 is used with the access sheath 104 to insert the distal end of the access sheath 104 into the blood vessel 901, the introducer 903 is removed from the access sheath 104, and an expansion-feature introducer is advanced through the access sheath 104 to expand the access sheath 104.

In another example, as shown in image 904B, the access sheath 104 can be advanced into the anatomy with the introducer 102 that includes an expansion feature. For example, the introducer 102 can be inserted into the access sheath 104 outside the patient, such that the distal end portion of the introducer 102 extends beyond the distal end of the access sheath 104 (in the configuration shown in the image 904B). The combined/coupled introducer 102 and the access sheath 104 can then be inserted into the blood vessel 901, so that at least the distal end of the access sheath 104 is disposed in the blood vessel 901.

FIG. 8-2 shows block 806 of the process 800. Images 906A and 906B of FIG. 9-2 show images corresponding to the block 806. At block 806, the process 800 includes dilating the access sheath using the introducer. For example, as shown in images 906A and 906B, the introducer 102 can be translated distally and/or proximally through a lumen in the access sheath 104 to radially expand one or more portions of the access sheath 104.

In one example, as shown in image 906A, the introducer 102 is implemented with a fixed and/or non-active expansion feature. The access sheath 104 is expanded by inserting and/or advancing the introducer 102 farther into the access sheath 104. This can expand at least a distal end portion of the access sheath 104 and/or other portions of the access sheath 104. Although discussed in the context of advancing the introducer 102 with the fixed expansion feature, the introducer 102 can alternatively, or additionally, be retracted through the access sheath 104.

In another example, as shown in image 906B, the introducer is implemented with an active and/or articulable expansion feature, wherein the expansion feature is enabled to expand a portion of the access sheath 104. In the illustrated example, the introducer 102 is translated distally such that the expansion feature extends beyond the distal end of the access sheath 104. The expansion feature is then enabled and/or activated on the introducer 102, such as by pulling a pull wire proximally to expand the expansion feature. The introducer 102 can then be retracted through the access sheath 104 to expand the access sheath 104. In other examples, the expansion feature is enabled while the expansion feature is located within the access sheath 104. Here, the introducer 102 can then be translated proximally and/or distally to expand the access sheath 104.

FIG. 8-3 shows blocks 808 and 810 of the process 800. Images 908 and 910 of FIG. 9-3 show images corresponding to the blocks 808 and 810. At block 808, the process 800 includes removing the introducer from the access sheath. For example, as shown in image 908, the introducer 102 can be retracted and/or moved proximally to remove the introducer 102 from the proximal end of the access sheath 104 (not shown in FIG. 9-3). In implementations where a locking mechanism is used, such as that illustrated in FIGS. 7A and 7B, the introducer 102 can be removed from the access sheath 104 once the introducer 102 is distally translated by at least a threshold distance to disable the locking mechanism. The introducer 102 can then be removed from the access sheath 104.

At block 810, the process 800 includes advancing a medical instrument through the access sheath. For example, as shown in image 910, a medical instrument 911 can be advanced through the access sheath 104 once the introducer 102 has been removed from the access sheath 104. The medical instrument 911 can include a delivery catheter and/or any other type of delivery/therapy device may be used in a medical procedure. In examples, the medical instrument 911 is navigated through the blood vessel 901 and/or other anatomy along the wire/guidewire 712 to reach a target location/anatomical feature.

In some illustrations, the process 800 is performed to deliver a prosthetic device to a target location, perform a therapy at the target location, and so on. For instance, the access sheath 104 can be inserted into an artery or vein to provide an access point for a delivery catheter or other therapy device to enter into the artery or vein. The access sheath 104 can be introduced into the artery or vein with a relatively small profile and/or diameter to prevent damage to the patient and/or access sheath 104. The introducer 102 can be then used to dilate and/or expand the access sheath 104. The introducer 102 can then be removed and a medical instrument can be inserted through the access sheath 104 and through the artery or vein to a target location to deploy a medical device, perform a therapy at the target location, etc. This can allow the access sheath 104 to be introduced into the artery or vein in a relatively small form and the access sheath 104 to be expanded when needed to provide larger access for the medical instrument. This can reduce, minimize, or prevent damage to the patient's tissue and/or reduce or minimize the push force needed to push the therapy device through the access sheath 104. Further, in some cases where the access sheath 104 is elastically expandable, the access sheath 104 can retract back to a smaller inner diameter once the expansion feature of the introducer 102, medical instrument, and/or other device passes through the access sheath 104. This can provide greater blood flow through the artery or vein during the procedure, since the access sheath 104 is only temporarily larger during device insertion.

FIG. 10 illustrates a cross-sectional view of an example configuration of the introducer 102 and the access sheath 104 according to one or more implementations. In some cases, the access sheath 104 can be advanced and/or inserted into the patient anatomy with the introducer 102 arranged and/or positioned in the configuration shown in FIG. 10. As illustrated, the introducer 102 is disposed within the access sheath 104 with the distal end portion of the introducer 102 extending beyond the distal end 306 of the access sheath 104. Here, the tapered portion 208 is positioned outside the distal end 306 of the access sheath 104 to provide an atraumatic point and/or end for the access sheath 104. The introducer 102 can be slidably received on the guidewire 712. In this example, the expansion feature 106 remains positioned within the access sheath 104 while the access sheath 104 is being advanced and/or inserted into the desired anatomy.

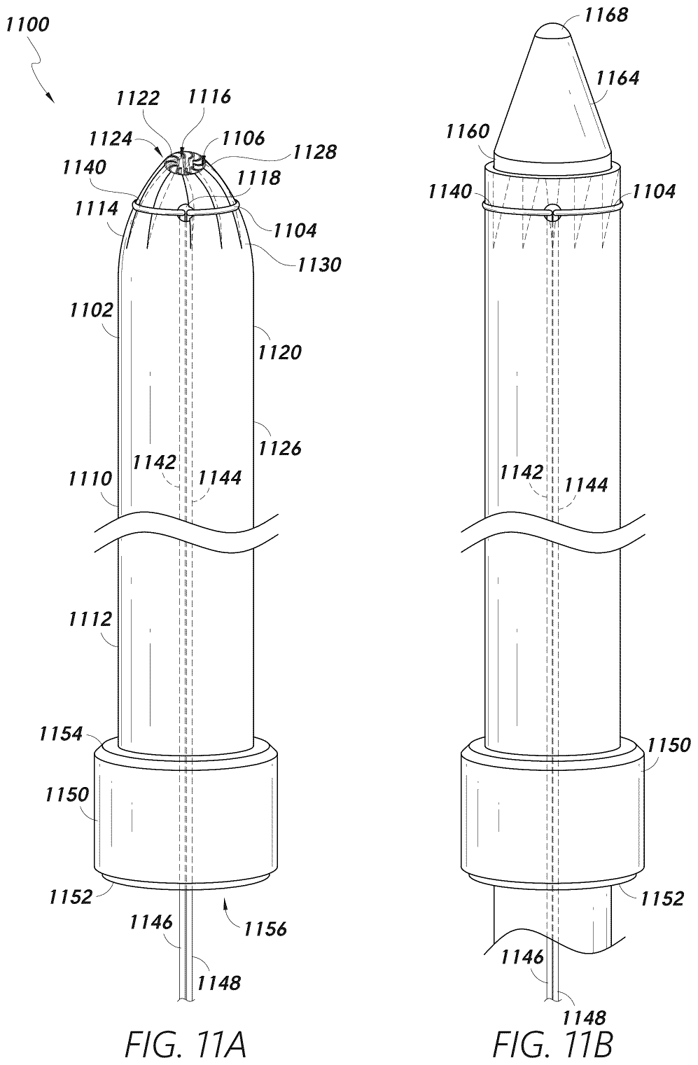

Medical access sheaths comprising expandable distal end portions are often used in minimally invasive procedures to facilitate delivery of medical devices. A medical access sheath comprising an expandable distal end portion can be inserted into a vessel, lumen and/or chamber prior to advancement therethrough of a delivery catheter and/or medical device. The expandable distal end portion can be kept in a closed and/or folded state while being advanced through an anatomical pathway. The expandable distal end portion of the sheath can subsequently assume an open and/or expanded state to allow passing of the delivery catheter and/or medical device through the expandable distal end portion. FIGS. 11A through 25 relate to various examples of expandable access sheaths and methods thereof. It will be understood that, in some instances, an expandable access sheath can comprise a combination of features from two or more of the expandable access sheaths described with reference to FIGS. 11A through 25. In some instances, one or more of the expandable access sheaths described with reference to FIGS. 11A through 25 can be used in combination with one or more of the introducers described with reference to FIGS. 1 and 2.

FIG. 11A and FIG. 11B are perspective views of an example of a medical access system 1100 comprising an expandable access sheath 1102 that includes a shaft 1110 with an expandable distal end portion 1114. FIG. 11A shows the expandable distal end portion 1114 in a closed state and FIG. 11B shows the expandable distal end portion 1114 in an open state. The expandable access sheath 1102 can comprise a sheath delivery lumen 1106 extending therethrough and configured to allow advancement therethrough of a medical device and/or delivery catheter configured to carry the medical device. The medical access system 1100 can include a cord 1104 comprising a medial portion 1140 configured to be disposed around a circumferential portion of the shaft 1110, such as a circumferential portion of the expandable distal end portion 1114. Remaining portions of the cord 1104 can pass from around the circumferential portion through a side shaft opening 1118 on the shaft 1110 and into the shaft delivery lumen 1116 and extend proximally from the side shaft opening 1118 along the shaft delivery lumen 1116 to facilitate manipulation of the cord 1104 by an operator. The operator can engage proximally disposed portions of the cord 1104 to facilitate control in the transformation of the expandable distal end portion 1114 from the open state to the closed state and/or vice versa. For example, the cord 1104 can be tensioned to facilitate maintaining the expandable distal end portion 1114 in the closed state. Tension in the cord 1104 can be relaxed to allow transformation of the expandable distal end portion 1114 from the closed state to the open state.

Referring to FIG. 11A, the expandable access sheath 1102 can comprise a hub 1150. The shaft 1110 can be disposed distally of the hub 1150. The shaft 1110 can be associated with a distal end 1154 of the hub 1150, for example a proximal portion 1112 of the shaft 1110 being coupled to the distal end 1154 of the hub 1150. The shaft 1110 can extend distally from the hub 1150, for example from the distal end 1154 of the hub 1150. The hub 1150 can comprise a hub delivery lumen 1156 extending therethrough. The hub delivery lumen 1156 can be in fluid communication with and/or be coaxial with the shaft delivery lumen 1116. The sheath delivery lumen 1106 can comprise the hub delivery lumen 1156 and the shaft delivery lumen 1116. The side shaft opening 1118 can extend through a wall portion 1120 of the shaft 1110 such that the side shaft opening 1118 is in fluid communication with the sheath delivery lumen 1106, including the shaft delivery lumen 1116 and the hub delivery lumen 1156. The side shaft opening 1118 can be on the expandable distal end portion 1114, for example extending through a wall portion 1120 of the expandable distal end portion 1114, such that remaining portions of the cord 1104 can be disposed through the side shaft opening 1118 and along at least a portion of the sheath delivery lumen 1106. The side shaft opening 1118 can be disposed on the same circumferential portion of the expandable distal end portion 1114 around which the cord 1104 is disposed. In some instances, at least a portion of the remaining portions of the cord 1104 can be disposed along the portion of the sheath delivery lumen 1106 proximal of the side shaft opening 1118. For example, at least a portion of the remaining portions of the cord 1104 can be disposed along the portion of the shaft delivery lumen 1116 proximal of the side shaft opening 1118 and through the hub delivery lumen 1156 such that a portion of the cord 1104 can extend proximally of a proximal end 1152 of the hub 1150. Alternatively, remaining portions of the cord 1104 can be disposed through another lumen extending along a portion of the shaft 1110 and the hub 1150.

As described herein, the expandable distal end portion 1114 of the shaft 1110 can be configured to be in the open state and the closed state. While the expandable distal end portion 1114 is in the closed state, a medial portion 1140 of the cord 1104 can be configured to be positioned around a circumferential portion of the expandable distal end portion 1114. Remaining portions of the cord 1104 can extend from around the circumferential portion through the side shaft opening 1118 on the shaft 1110 and into the shaft delivery lumen 1116. For example, the cord 1104 can comprise a first cord tail portion 1142 and a second cord tail portion 1144. The medial cord portion 1140 can be between the first and second cord tail portions 1142, 1144. In some instances, while the expandable distal end portion 1114 is in the closed state, the medial cord portion 1140 can be configured to be disposed around a circumferential portion of the expandable distal end portion 1114. The medial cord portion 1140 can be disposed around a circumferential portion of the expandable distal end portion 1114 that is externally oriented such the first and second cord tail portions 1142, 1144 can pass from an external surface 1126 of the shaft 1110 through the side shaft opening 1118 into the shaft delivery lumen 1116. The first and second cord tail portions 1142, 1144 can be configured to extend through the side shaft opening 1118 on the expandable distal end portion 1114 of the shaft 1110, into the shaft delivery lumen 1116 and extend proximally along at least a portion of the shaft delivery lumen 1116 such that an operator can manipulate a proximal portion 1146, 1148 of each of the first and second cord tail portions 1142, 1144. Corresponding portions of the first and second cord tail portions 1142, 1144 can be disposed along the portion of the shaft delivery lumen 1116 proximal of the side shaft opening 1118. In some instances, corresponding portions of the first and second cord tail portions 1142, 1144 can be disposed through at least a portion of the hub delivery lumen 1156. The first and second cord tail portions 1142, 1144 can extend proximally from the shaft delivery lumen 1116 into the hub delivery lumen 1156, and along at least a portion of the hub delivery lumen 1156. In some instances, the proximal portions 1146, 1148 of the first and second cord tail portions 1142, 1144 can be coupled to and/or secured to the hub 1150 while the expandable distal end portion 1114 is in the closed state or the open state. Securing the proximal portions 1146, 1148 of the first and second cord tail portions 1142, 1144 to the hub 1150 can facilitate maintaining the expandable distal end portion 1114 in the closed state or the open state.

The medial portion 1140 of the cord 1104 can be disposed around a circumferential portion of the expandable distal end portion 1114 proximal of the distal end 1122 of the shaft 1110. In some instances, the circumferential portion can be at about a medial portion along a longitudinal dimension of the expandable distal end portion 1114. In some instances, the longitudinal dimension can be parallel or substantially parallel to a longitudinal axis of the shaft 1110. Alternatively, the longitudinal dimension can be a longitudinal dimension along an externally oriented surface of the expandable distal end portion 1114. For example, the longitudinal dimension can extend between a proximal end 1130 and a distal end 1128 of the expandable distal end portion 1114. The distal end 1128 of the expandable distal end portion 1114 can be the distal end 1122 of the shaft 1110. The circumferential portion can be at about midway between the proximal end 1128 and the distal end 1130 of the expandable distal end portion 1114. The circumferential portion can be at a position on the expandable distal end portion 1114 that facilitates maintaining the expandable distal end portion 1114 in the closed state while the cord 1104 is tensioned.

In some instances, at least a portion of the medical access sheath 1102, including at least a portion of the shaft 1110, can be inserted into an anatomical vessel and/or lumen and advanced along an anatomical pathway while the expandable distal end portion 1114 is in the closed state. In some instances, while the shaft 1110 is advanced to a target site, tension can be maintained in the cord 1104 to facilitate maintaining the expandable distal end portion 1114 in the closed state. In some instances, the proximal portions 1146, 1148 of the first and second cord tail portions 1142, 1144 can be secured to the hub 1150 to maintain desired tension on the cord 1104, including tensioning of the first and second cord tail portions 1142, 1144 such that the expandable distal end portion 1114 remains in the closed state.

The expandable distal end portion 1114 can expand and/or unfold to transform the expandable distal end portion 1114 to the open state. In some examples, the expandable distal end portion 1114 can comprise a plurality of folds and/or pleats. The expandable distal end portion 1114 can comprise a folded configuration in the closed state. The plurality of folds and/or pleats can unfold such that the expandable distal end portion 1114 can assume the open state.

Referring to FIG. 11B, the expandable distal end portion 1114 can unfold and/or expand to assume the open state. FIG. 11B shows at least a portion of an introducer 1160 disposed within the sheath delivery lumen 1106 of the medical access sheath 1102. A distal portion 1164 of the introducer 1160 can be advanced through the expandable distal end portion 1114 and out of a distal opening 1124 at a distal end of the shaft 1110 such that at least a portion of the introducer 1160, such as a distal end 1168, is disposed distally of the distal end 1122 of the shaft 1110. Tension in the cord 1104 can be reduced and/or relaxed to facilitate expansion of the expandable distal end portion 1114 as the introducer 1160 is advanced through the expandable distal end portion 1114. For example, at least a portion of the introducer 1160 can be advancement through the hub delivery lumen 1156 and the shaft delivery lumen 1116. Advancement of the introducer 1160 through a corresponding portion of the shaft delivery lumen 1116 extending through the expandable distal end portion 1114 can cause the expandable distal end portion 1114 to expand and/or unfold to assume the open state. For example, the cord 1104 can be allowed to have some slack as the introducer 1160 is advanced such that the expandable distal end portion 1114 can expand and/or unfold to assume the open state. Additional portions of the cord 1104 can be allowed to be positioned around the circumferential portion of the expandable distal end portion 1114 as it expands and/or unfolds. For example, while the expandable distal end portion 1114 is in the open state, portions of the first and second cord tail portions 1142, 1144 adjacent to the medial cord portion 1140 can be positioned around the circumferential portion of the expandable distal end portion 1114.

The cord 1104 can remain while the introducer 1160 is advanced to its desired position. The first and second cord tail portions 1142, 1144 can remain in the shaft delivery lumen 1116. In some instances, while the expandable distal end portion 1114 is in the open state, proximal portions 1146, 1148 of the first and second cord tail portions 1142, 1144 can be secured to the hub 1150. The cord 1104 can be tensioned to facilitate transforming the expandable distal end portion 1114 to the closed state from the open state. For example, an operator can pull on the cord 1104, such as proximal portions 1146, 1148 of the first and second cord tail portions 1142, 1144, to facilitate folding of the expandable distal end portion 1114 so as to transform the expandable distal end portion 1114 from the open state to the closed state. In some instances, the expandable distal end portion 1114 can be transformed to the closed state before the medical access sheath is retracted. The expandable distal end portion 1114 can be maintained in the closed state while the medical access sheath 1102 is retracted and withdrawn. For example, after the expandable distal end portion 1114 assumes the closed state, the cord 1104 can be kept in a tensioned state to facilitate maintaining the expandable distal end portion 1114 in the closed state.

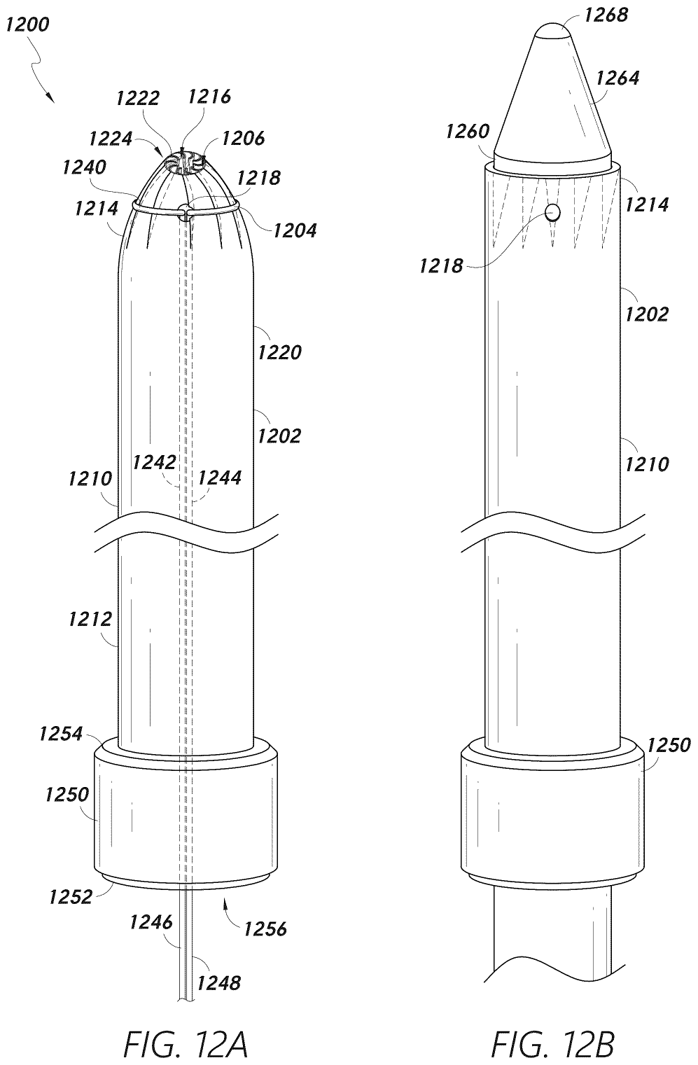

FIG. 12A and FIG. 12B are perspective views of another example of a medical access system 1200. An expandable access sheath 1202 of the medical access system 1200 can include a shaft 1210 comprising an expandable distal end portion 1214. The medical access system 1200 can comprise a cord 1204 having a portion configured to be circumferentially positioned around the expandable distal end portion 1214 while the expandable distal end portion 1214 is in a closed state. The cord 1204 can be removed from the expandable access sheath 1202 after the expandable distal end portion 1214 assumes an open state. FIG. 12A shows the expandable distal end portion 1214 in the closed state and FIG. 12B shows the expandable distal end portion 1214 in the open state.

The medical access sheath 1202 can have one or more features of the medical access sheath 1102. For example, the expandable access sheath 1202 can comprise a hub 1250. The shaft 1210 can be disposed distally of the hub 1250. A proximal portion 1212 of the shaft 1210 can be associated with a distal end 1254 of the hub 1250, for example being coupled to the distal end 1254 of the hub 1250. The shaft 1210 can extend distally from the hub 1250, for example from the distal end 1254 of the hub 1250. The expandable access sheath 1202 can comprise a sheath delivery lumen 1206 extending therethrough and configured to allow advancement therethrough of a medical device and/or delivery catheter configured to carry the medical device. The sheath delivery lumen 1206 can comprise a hub delivery lumen 1256 extending through the hub 1250, and a shaft delivery lumen 1216 extending through the shaft 1210 and in fluid communication with and/or be coaxial with the hub delivery lumen 1256.

The cord 1204 can comprise a medial portion 1240 configured to be disposed around a circumferential portion of the expandable distal end portion 1214. The cord 1204 can comprise a first and a second cord tail portion 1242, 1244, the medial cord portion 1240 being between the first and second cord tail portions 1242, 1244. The first and second cord tail portions 1242, 1244 can pass from around the circumferential portion through a side shaft opening 1218 of a wall portion 1220 of the shaft 1210 and into the shaft delivery lumen 1216, extend proximally from the side shaft opening 1218 along the shaft delivery lumen 1216 to facilitate manipulation of the cord 1204 by an operator. While the expandable distal end portion 1214 is in the closed state, proximal portions 1246, 1248 of the first and second cord tail portions 1242, 1244 can be secured to the expandable access sheath 1202, including the hub 1250 to maintain tension in the cord 1204. In some instances, the first and second cord tail portions 1242, 1244 can comprise portions, including proximal portions 1246, 1248, disposed proximally of a proximal end 1252 of the hub 1250, such as to facilitate securing the first and second cord tail portions 1242, 1244 to the hub 1250 and/or manipulation by the operator. Tension in the cord 1204 can be relaxed to allow transformation of the expandable distal end portion 1214 from the closed state to the open state.

Referring to FIG. 12B, the cord 1204 can be removed from the expandable access sheath 1202 after the expandable distal end portion 1214 assumes the open state. At least a portion of an introducer 1260 can be advanced through the sheath delivery lumen 1206 after removal of the cord 1204. For example, a distal portion 1264 of the introducer 1260 can be advanced through the hub delivery lumen 1256 and the shaft delivery lumen 1216, and through a distal opening 1224 at a distal end 1222 of the shaft 1210, such that a distal end 1268 of the introducer 1260 is disposed distally of the distal end 1222 of the shaft 1210. Advancing at least a portion of the introducer 1260 distally through the distal opening 1224 can transform the expandable distal end portion 1214 to the open state, such as by causing the expandable distal end portion 1114 to expand and/or unfold. For example, disposing the distal portion 1264 of the introducer 1260 distally of the distal end 1222 of the shaft 1210 can transform the expandable distal end portion 1214 to the open state.

It will be understood that although the shafts of FIGS. 11A, 11B, 12A and 12B are shown as including one side shaft opening, a first side shaft opening and a second side shaft opening can be included. For example, the first cord tail portions 1142, 1242 can pass through a respective first shaft opening and the second cord tail portions 1144, 1244 can pass through a respective second shaft opening.

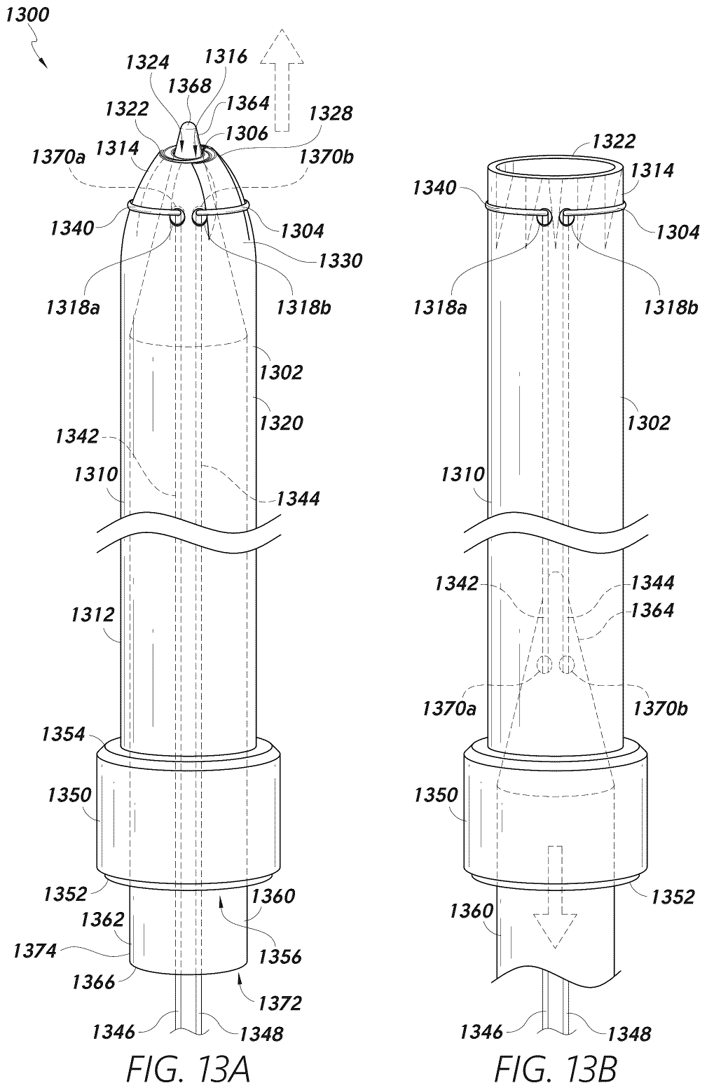

In some instances, an introducer can be preloaded within an expandable access sheath. For example, at least a portion of the introducer can be disposed within the expandable access sheath while the expandable access sheath is advanced along an anatomical pathway to a target location. FIGS. 13A and 13B are perspective views of an example of a medical access system 1300 comprising an introducer 1360 preloaded within an expandable access sheath 1302. While preloaded, a portion of the introducer 1360, such as at least a portion of a distal portion 1364, can be extending through a distal opening 1324 at a distal end 1322 of the shaft 1310. For example, a distal end 1368 of the introducer 1360 can be disposed distally of the distal end 1322 of the shaft 1310. The expandable access sheath 1302 can comprise a shaft 1310 with an expandable distal end portion 1314 configured to be in an open state and a closed state. FIG. 13A shows the expandable distal end portion 1314 in the closed state, and FIG. 13B shows the expandable distal end portion 1314 in the open state. The medical access system 1300 can include a cord 1304 comprising a portion configured to be disposed around a circumferential portion of the expandable distal end portion 1314 of the expandable access sheath 1302. Remaining portions of the cord 1304 can be disposed through a side introducer opening on the introducer 1360 and a side shaft opening on the shaft 1310. In some instances, the side shaft opening can comprise a first side shaft opening 1318a and a second side shaft opening 1318b. In some instances, the side introducer opening can comprise a first side introducer opening 1370a and a second side introducer opening 1370b. For example, remaining portions of the cord 1304 can pass through respective side introducer openings 1370a, 1370b on the introducer 1360 and side shaft openings 1318a, 1318b on the expandable distal end portion 1314. Portions of the cord 1304 can extend along a corresponding portion of a central lumen 1372 of the introducer 1360 such that proximal portions of the cord 1304 can be manipulated by an operator to facilitate transformation of the expandable distal end portion 1314 from the closed state to the open state and/or vice versa.

Referring to FIG. 13A, the shaft 1310 can be disposed distally of and coupled to a hub 1350. The shaft 1310 comprising the expandable distal end portion 1314 can be associated with a distal end 1354 of the hub 1350, for example a proximal portion 1312 of the shaft 1310 being coupled to the distal end 1354 of the hub 1350. The shaft 1310 can extend distally from the hub 1350, for example from the distal end 1354 of the hub 1350. A shaft delivery lumen 1316 can extend through the shaft 1310, and be in fluid communication with a hub delivery lumen 1356 extending through the hub 1350. The side shaft openings 1318a, 1318b can be on a wall portion 1320 of the shaft 1310 and be in fluid communication with shaft the delivery lumen 1316. The expandable access sheath 1302 can comprise a sheath delivery lumen 1306 comprising the shaft delivery lumen 1316 and the hub delivery lumen 1356.

The introducer 1360 can comprise the central lumen 1372 extending along at least a portion thereof. For example, the central lumen 1372 can extend from a proximal end 1366 to a distal portion 1364 of the introducer 1360. The first and the second side introducer opening 1370a, 1370b can be on the distal portion 1364 of the introducer 1360. The side introducer openings 1370a, 1370b can extend through a wall portion 1374 of the introducer 1360 such that the side introducer openings 1370a, 1370b are in fluid communication with the central lumen 1372. While the expandable distal end portion 1314 is in the closed state, at least a portion of the introducer 1360 can be configured to be slidably received within the shaft delivery lumen 1316 such that the side introducer openings 1370a, 1370b can be configured to be within the shaft delivery lumen 1316. In some instances, the side introducer openings 1370a, 1370b and the side shaft openings 1318a, 1318b can be aligned with one another while the expandable distal end portion 1314 is in the closed state, such as while the introducer 1360 is preloaded within the within the expandable access sheath 1302. For example, the first side shaft opening 1318a can be aligned with the first side introducer opening 1370a, and the second side shaft opening 1318b can be aligned with the second side introducer opening 1370b.

The cord 1304 can comprise a medial portion 1340 between a first and second cord tail portion 1342, 1344. The medial cord portion 1340 can be configured to be disposed around the circumferential portion of the expandable distal end portion 1314 while the expandable distal end portion 1314 is in the closed state. Remaining portions of the cord 1304 can pass from around the circumferential portion through the side shaft openings 1318a, 1318b and the side introducer openings 1370a, 1370b, into the central lumen 1372 and extend proximally from the side introducer openings 1370a, 1370b along the central lumen 1372 to facilitate manipulation of the cord 1304 by an operator. The first cord tail portion 1342 can be disposed through the first side shaft opening 1318a and the first side introducer opening 1370a. The second cord tail portion 1344 can be disposed through the second side shaft opening 1318b and the second side introducer opening 1370b. Respective portions of the first and second cord tail portions 1342, 1344 can be disposed along at least a portion of the central lumen 1372 of the introducer 1360. The cord 1304 can be tensioned to facilitate maintaining the expandable distal end portion 1314 in the closed state. Tension in the cord 1304 can be relaxed to allow transformation of the expandable distal end portion 1314 from the closed state to the open state. Proximal portions 1346, 1348 of the first and second cord tail portions 1342, 1344 can extend proximally from a proximal end 1366 of the introducer 1360 to facilitate manipulation thereof by an operator. For example, a proximal portion 1362 of the introducer 1360 can be disposed proximally of a proximal end 1352 of the hub 1350 and proximal portions 1346, 1348 of the first and second cord tail portions 1342, 1344 can be disposed proximally of the proximal end 1366 of the introducer 1360.

The circumferential portion around which the medial cord portion 1340 is disposed can be at a position that facilitates maintaining the expandable distal end portion 1314 in the closed state while the cord 1304 is tensioned. In some instances, the position of the circumferential portion can be at about a medial portion along a longitudinal dimension of the expandable distal end portion 1314. In some instances, the longitudinal dimension can be parallel or substantially parallel to a longitudinal axis of the shaft 1310. Alternatively, the longitudinal dimension can be a longitudinal dimension along an externally oriented surface of the expandable distal end portion 1314. For example, the longitudinal dimension can extend between a proximal end 1330 and a distal end 1328 of the expandable distal end portion 1314. The distal end 1330 of the expandable distal end portion 1314 can be the distal end 1322 of the shaft 1310. The circumferential portion can be at about midway between the proximal end 1330 and the distal end 1328 of the expandable distal end portion 1314.

In some instances, the introducer 1360 can be translated distally relative to the expandable access sheath 1302 to open the expandable distal portion 1314. For example, advancing the distal portion 1364 of the introducer 1360 through the distal opening 1324 and disposing the distal portion 1364 distally of the distal end 1322 of the shaft 1310 can cause the expandable distal end portion 1314 to expand and/or unfold. Referring to FIG. 13B, in some instances, the cord 1304 can remain after the expandable distal end portion 1314 assumes the open state. For example, the medial cord portion 1340 can remain disposed around the circumferential portion of the expandable distal end portion 1314 after the expandable distal end portion 1314 transforms to the open state from the closed state. After the expandable distal end portion 1314 assumes the open state, the introducer 1360 can be translated proximally relative to the expandable access sheath 1302 to withdraw the introducer 1360 from within the shaft delivery lumen 1316. For example, the first and second side introducer openings 1370a, 1370b can be configured to slide proximally along at least a portion of the first and second cord tail portions 1342, 1344 as the introducer 1360 is translated proximally. As the introducer 1360 is translated proximally and withdrawn, the first and second side introducer openings 1370a, 1370b can slide along respective portions of the first and second cord tail portions 1342, 1344 as the respective portions of the first and second cord tail portions 1342, 1344 pass from the central lumen 1372 into the shaft delivery lumen 1316.

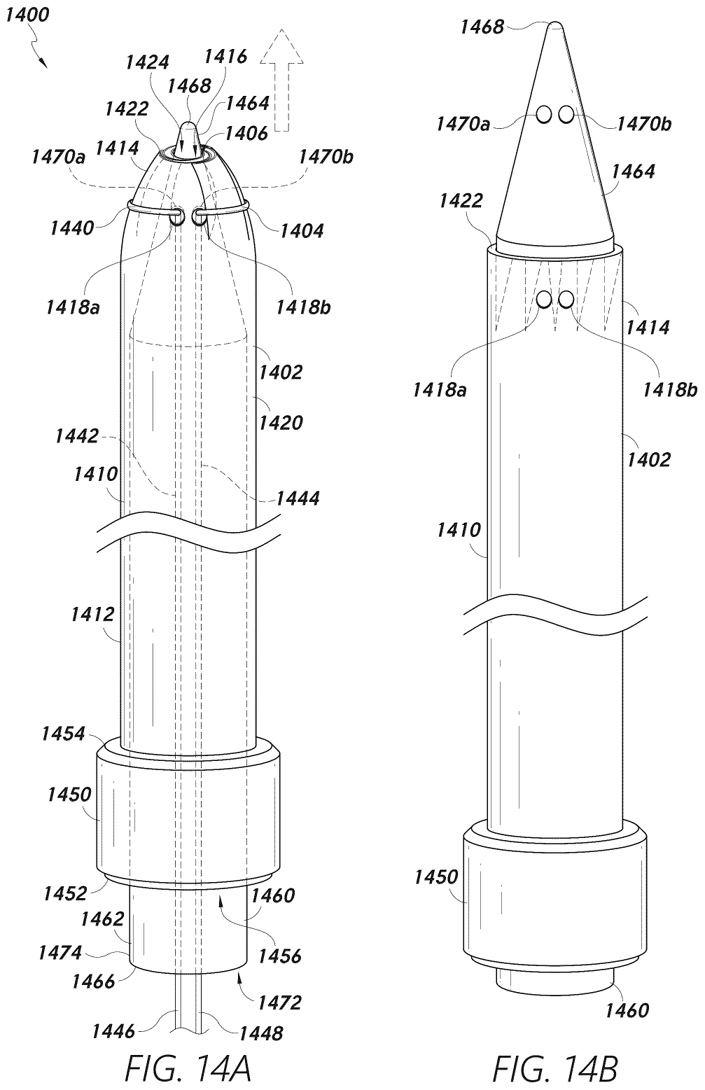

FIGS. 14A and 14B are perspective views of another example of a medical access system 1400 comprising an introducer 1460 preloaded within an expandable access sheath 1402. The expandable access sheath 1402 can comprise a shaft 1410 with an expandable distal end portion 1414 configured to be in an open state and a closed state. A portion of a cord 1404 can be configured to be positioned around a circumferential portion of the expandable distal end portion 1414 in the closed state. FIG. 14A shows an expandable distal end portion 1414 of the expandable access sheath 1402 in a closed state, and FIG. 14B shows the expandable distal end portion 1414 in an open state, where the cord 1404 has been removed from the expandable access sheath 1402. As described in further detail herein, in some instances, the cord 1404 can be removed to facilitate expansion and/or opening of the expandable distal end portion 1414.

The medical access system 1400 can comprise one or more other features of the medical access system 1300 described with reference to FIGS. 13A and 13B. In some instances, the medical access system 1400 can comprise the other features of the medical access system 1300 described with reference to FIGS. 13A and 13B. For example, the shaft 1410 can be disposed distally of and coupled to a hub 1450. A proximal portion 1412 of the shaft 1410 can be associated with a distal end 1454 of the hub 1450, for example a proximal portion 1412 of the shaft 1410 being coupled to the distal end 1454 of the hub 1450. The shaft 1410 can extend distally from the hub 1450, for example from the distal end 1454 of the hub 1450. A sheath delivery lumen 1406 can comprise a shaft delivery lumen 1416 extending through the shaft 1410, and a hub delivery lumen 1456 extending through the hub 1450. First and second side shaft openings 1418a, 1418b can be on a wall portion 1420 of the shaft 1410 and be in fluid communication with shaft the delivery lumen 1416. An introducer 1460 can be preloaded within the sheath delivery lumen 1406 while the expandable distal end portion 1414 is in the closed state. While preloaded, a portion of the introducer 1460, such as at least a portion of a distal portion 1464, can be extending through a distal opening 1424 at a distal end 1422 of the shaft 1410. For example, a distal end 1468 of the introducer 1460 can be disposed distally of the distal end 1422 of the shaft 1410. The introducer 1460 can comprise a central lumen 1472 extending along at least a portion thereof. A first and a second side introducer opening 1470a, 1470b can be on the distal portion 1464 of the introducer 1460. The side introducer openings 1470a, 1470b can extend through a wall portion 1474 of the introducer 1460 and be in fluid communication with the central lumen 1472. The side introducer openings 1470a, 1470b and the side shaft openings 1418a, 1418b can be aligned with one another while the expandable distal end portion 1414 is in the closed state. For example, the first side shaft opening 1418a can be aligned with the first side introducer opening 1470a, and the second side shaft opening 1418b can be aligned with the second side introducer opening 1470b.

The cord 1404 can comprise a medial portion 1440 between a first and second cord tail portion 1442, 1444. The medial cord portion 1440 can be configured to be disposed around the circumferential portion of the expandable distal end portion 1414 while the expandable distal end portion 1414 is in the closed state. The first cord tail portion 1442 can pass from around the circumferential portion through the first side shaft opening 1418a and the first side introducer opening 1470a into the central lumen 1472. The second cord tail portion 1444 can pass from around the circumferential portion through the second side shaft opening 1418b and the second side introducer opening 1470b into the central lumen 1472. Respective portions of the first and second cord tail portions 1442, 1444 can be disposed along at least a portion of the central lumen 1472 of the introducer 1460. Proximal portions 1446, 1448 of the first and second cord tail portions 1442, 1444 can extend proximally from a proximal end 1466 of the introducer 1460 to facilitate manipulation thereof by an operator. For example, a proximal portion 1462 of the introducer 1460 can be disposed proximally of a proximal end 1452 of the hub 1450 and proximal portions 1446, 1448 of the first and second cord tail portions 1442, 1444 can be disposed proximally of the proximal end 1466 of the introducer 1460.

Referring to FIG. 14B, the cord 1404 can be removed from the expandable access sheath 1402 to facilitate transformation of the expandable distal end portion 1414 to the open state or after the expandable distal end portion 1414 assumes the open state. The cord 1404 can be configured to be removed from around the expandable access sheath 1402 and from the central lumen 1472 while the at least a portion of the introducer 1460 is received within the sheath delivery lumen 1406 to allow the expandable distal end portion 1414 to assume the open state. In some instances, the introducer 1460 can be advanced relative the expandable access sheath 1402 to facilitate transformation of the expandable distal end portion 1414 to the open state. For example, as shown in FIG. 14B, the introducer 1460 is further distally displaced relative to the expandable access sheath 1402 than is shown in FIG. 14A. For example, after the cord 1404 is removed, the introducer 1460 can be translated distally relative to the expandable access sheath 1402 to open the expandable distal portion 1414. Advancing the distal portion 1464 of the introducer 1460 through the distal opening 1424 and disposing the distal portion 1464 distally of the distal end 1422 of the shaft 1410 can cause the expandable distal end portion 1414 to expand and/or unfold.

It will be understood that although the shafts of FIGS. 13A, 13B, 14A and 14B are shown as including two side shaft openings, the shafts can each include one shaft opening configured to allow passage therethrough of both cord tail portions. For example, both the first cord tail portions 1142, 1242 and the second cord tail portions 1144, 1244 can pass through the same side shaft opening on the respective shaft.

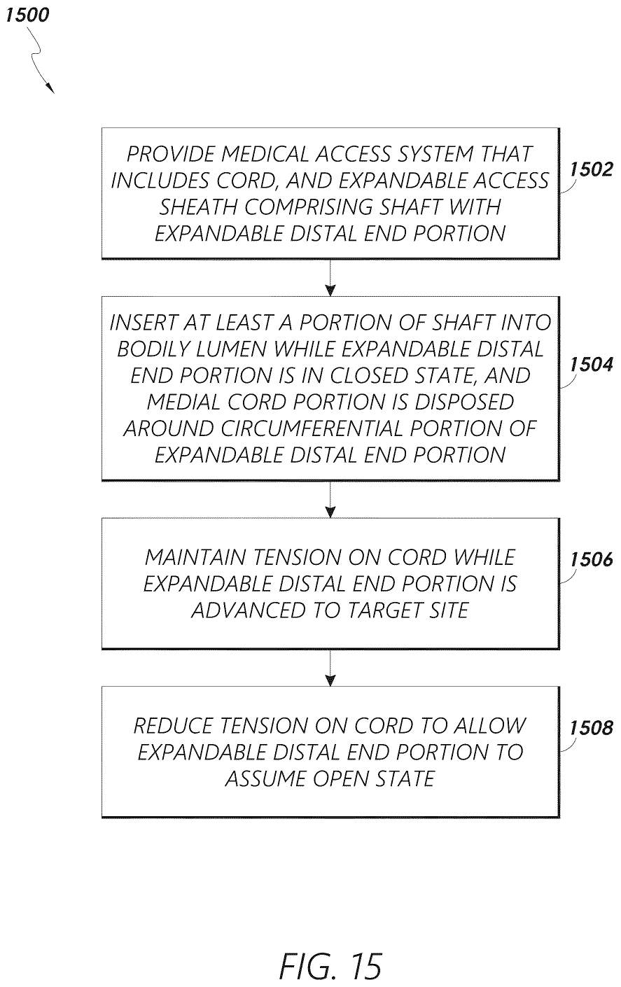

FIG. 15 is a process flow diagram of an example of a delivery process 1500 for delivering a medical device using a medical access system as described herein. For example, the delivery process 1500 can be applicable to one or more of the medical access systems 1100, 1200, 1300, 1400 described with reference to FIGS. 11A, 11B, 12A, 12B, 13A, 13B, 14A and 14B. In block 1502, the process can involve providing a medical access system. In some instances, the medical access system can include an expandable access sheath comprising a shaft extending distally from a hub, where the shaft comprises an expandable distal end portion. A first and a second side shaft opening can be on the expandable distal end portion and in fluid communication with a shaft delivery lumen extending through the shaft. The medical access system can comprise a cord comprising a first cord tail portion, a second cord tail portion, and a medial cord portion between the first and second cord tail portions.

In block 1504, the process can involve inserting at least a portion of the shaft into a bodily lumen while the expandable distal end portion is in a closed state. While the expandable distal end portion is in the closed state, the medial cord portion can be disposed around a circumferential portion of the expandable distal end portion. The first and second cord tail portions can extend through a respective one of the first and second side shaft openings. For example, corresponding portions of the first and second cord tail portions can be disposed within the shaft delivery lumen.

In block 1506, the process can involve maintaining tension in the cord. The tension on the cord can be maintained so as to maintain the expandable distal end portion in the closed state. In some instances, the expandable distal end portion can be maintained in the closed state while the expandable distal end portion is advanced along an anatomical pathway to a target site.

In block 1508, the process can involve reducing the tension on the cord to allow the expandable distal end portion to assume an open state. For example, reducing tension on the cord can allow the expandable distal end portion to expand and/or unfold such that a portion of at least one of the first and second cord tail portions can be positioned over the circumferential portion of the expandable distal end portion.

In some instances, the cord can be removed after the expandable distal end portion assumes the open state. In some instances, the cord can be removed to facilitate allowing the expandable distal end portion to assume the open state. In some instances, the process can involve passing the medial cord portion and then the second tail cord portion through the first and second side shaft openings and into the shaft delivery lumen, as the first tail cord portion is translated proximally to remove the cord and allow the expandable distal end portion to assume the open state. In some instances, the process can involve passing the medial cord portion and then the first tail cord portion through the first and second side shaft openings and into the shaft delivery lumen as the second tail cord portion is translated proximally to remove the cord and allow the expandable distal end portion to assume the open state.

In some instances, an introducer can be provided that is at least partially received in the shaft delivery lumen of the shaft. The introducer can comprise a central lumen extending therethrough. A first side introducer opening and second side introducer opening can be on a portion of the introducer, including on a distal portion of the introducer. The first and second side introducer openings can be in fluid communication with the central lumen. In some instances, while the expandable distal end portion is advanced to a target site, the introducer can be preloaded within the expandable access sheath. For example, at least a portion of the introducer can be received within the shaft delivery lumen. A portion of the distal portion of the introducer, including a distal end of the introducer, can be disposed distally of a distal end of the shaft while the introducer is preloaded within the delivery lumen of the expandable access sheath. The first and second side introducer openings can be disposed within the shaft delivery lumen. For example, the first and second cord tail portions can extend through a respective one of the first and second side shaft openings and then a respective one of the first and second side introducer openings. Corresponding portions of the first and second cord tail portions can extend along the central lumen of the introducer. For example, corresponding portions of the first and second cord tail portions can be disposed with the portion of the central lumen proximal of the first and second side introducer openings.

In some instances, the introducer can be translated proximally relative to the expandable access sheath to withdraw the introducer from within the delivery lumen of the expandable access sheath. For example, the introducer can be translated proximally relative to the expandable access sheath to withdraw the introducer from within the shaft delivery lumen. The introducer can be further translated proximally relative to the expandable access sheath to withdraw the introducer from within the hub delivery lumen. While the introducer is translated proximally, the first and second side introducer openings can slide proximally along at least a portion of the first and second cord tail portions extending through the delivery lumen of the expandable access sheath, such as the shaft delivery lumen and/or hub delivery lumen. The cord can remain in place while the introducer is withdrawn from the expandable access sheath. For example, the medial cord portion can remain around the circumferential portion of the expandable distal end portion while the introducer is translated proximally.

In some instances, the cord can be removed from the expandable access sheath before the introducer is withdrawn. In some instances, the cord can be removed from the expandable access sheath to facilitate transformation of the expandable distal end portion to the open state. For example, the process can involve passing the medial cord portion and then the second tail cord portion through the first and second side shaft openings and the first and second side introducer openings and the central lumen as the first tail cord portion is translated proximally to remove the cord and allow the expandable distal end portion to assume the open state. Alternatively, the process can involve passing the medial cord portion and then the first tail cord portion through the first and second side shaft openings and the first and second side introducer openings and the central lumen as the second tail cord portion is translated proximally to remove the cord and allow the expandable distal end portion to assume the open state.

In some instances, a position of the cord can be secured to facilitate maintaining the expandable distal end portion in the open state or the closed state. Proximal portions of the cord can be secured to a portion of the expandable access sheath. In some instances, the process can involve securing a proximal portion of the first tail cord portion and a proximal portion of the second tail cord portion to the hub of the expandable access sheath to maintain the expandable distal end portion in the closed state or the open state.

In some instances, leaving the cord in place after the expandable distal end portion assumes the open state can facilitate transformation of the expandable distal end portion back to the closed state before withdrawing the shaft from the patient. In some instances, the process can involve tensioning the cord to transform the expandable distal end portion of the shaft from the open state to the closed state. Tensioning the cord can cause a force to be exerted upon the circumferential portion of the expandable distal end portion, such as by the medial cord portion. Force exerted on the circumferential portion of the expandable distal end portion can lead to a reduction in a lateral cross-sectional size of a corresponding portion of the expandable distal end portion. For example, force exerted on the circumferential portion of the expandable distal end portion can cause the expandable distal end portion can transform back to the closed state. In some instances, the process can involve translating the shaft proximally to withdraw the shaft from the bodily lumen, such as after the expandable distal end portion assumes the closed state.

In some instances, at least a portion of a medical device can be advanced through the delivery lumen of the expandable access sheath. The medical device can be carried on and/or within a delivery catheter. For example, at least a portion of a delivery catheter configured to receive the medical device can be advanced through the shaft delivery lumen. In some instances, the delivery catheter can be first advanced through the shaft delivery lumen. The medical device can subsequently be deployed from the delivery catheter. For example, at least a portion of the medical device can be advanced through the delivery catheter to be deployed from the delivery catheter.

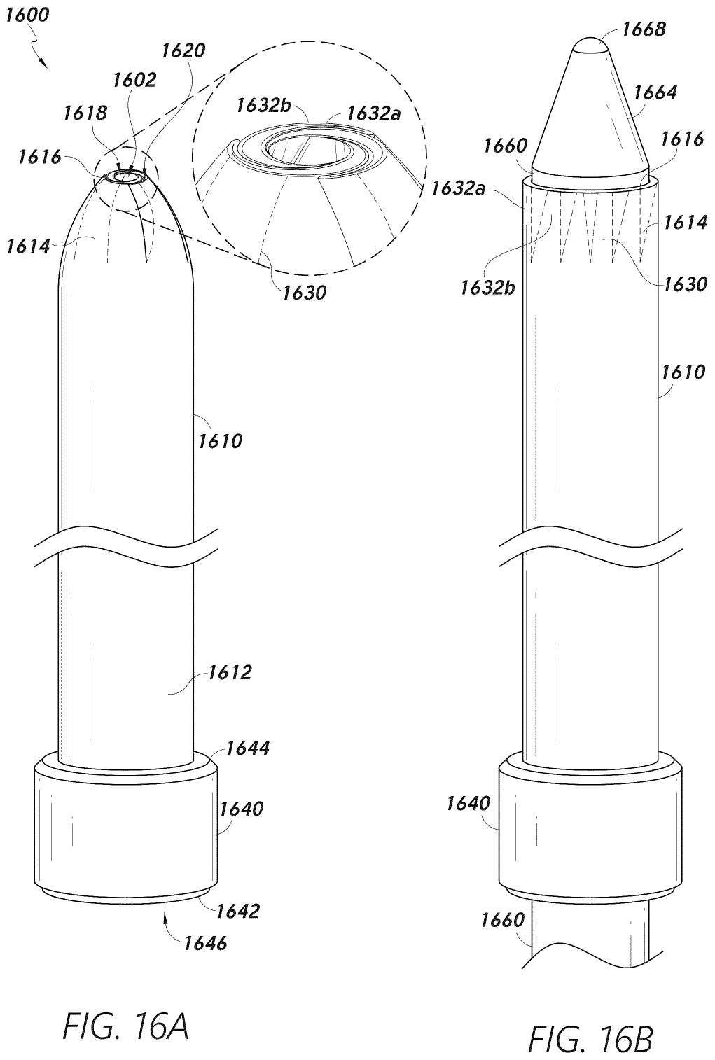

FIGS. 16A and 16B are perspective views of an example of an expandable access sheath 1600 that includes a shaft 1610 comprising an expandable distal end portion 1614 having a plurality of pleats 1630, where respective pairs of pleat portions of each of the pleats 1630 can be configured to be attracted to one another to maintain the expandable distal end portion 1614 in a closed state. FIG. 16A shows the expandable distal end portion 1614 of the expandable access sheath 1600 in the closed state and FIG. 16B shows the expandable distal end portion 1614 in an open state. The plurality of pleats 1630 of the expandable distal end portion 1614 can be configured to be in a folded configuration while the expandable distal end portion 1614 is in the closed state. Each of the plurality of pleats 1630 can comprise a pair of pleat portions configured to be oriented toward one another while the plurality of pleats 1630 are in the folded configuration. For example, a first pleat portion 1632a and a second pleat portion 1632b can be oriented toward and be attracted to one another while the plurality of pleats 1630 are in the folded configuration. Attraction between the pair of pleat portions of each pleat can be configured to maintain the expandable distal end portion 1614 in the folded configuration.

The expandable access sheath 1600 can comprise a sheath delivery lumen 1602 extending therethrough and configured to allow advancement of a medical device and/or delivery catheter configured to carry the medical device through the sheath delivery lumen 1602. The expandable access sheath 1600 can comprise a hub 1640 associated with a proximal portion 1612 of the shaft 1610. The proximal portion 1612 of the shaft 1610 can be coupled to the distal end 1644 of the hub 1640. The shaft 1610 can extend distally from the hub 1640, including from the distal end 1644 of the hub 1640. The shaft 1610 can comprise a shaft delivery lumen 1616 extending therethrough. The shaft delivery lumen 1616 can be in communication with and/or be coaxial with the hub delivery lumen 1646. The hub 1640 can comprise a hub delivery lumen 1646 extending therethrough, from a proximal end 1642 to a distal end 1644 thereof. The sheath delivery lumen 1602 can comprise the shaft delivery lumen 1616 and the hub delivery lumen 1646.

At least a portion of a medical instrument can be advanced through the sheath delivery lumen 1602 to facilitate transformation of the expandable distal end portion 1614 to the open state. Referring to FIG. 16B, the expandable distal end portion 1614 is shown in the open state. The expandable distal end portion 1614 can be in an unfolded configuration while in the open state. At least a portion of an introducer 1660 is shown as being disposed through the sheath delivery lumen 1602. At least a portion of the introducer 1660 can be advanced through the hub delivery lumen 1646 and shaft delivery lumen 1616 such that as a distal portion 1664 of the introducer 1660 is advanced through a corresponding portion of the shaft delivery lumen 1616 extending through the expandable distal end portion 1614, the introducer 1660 can push apart the plurality of pleats 1630. Advancing the introducer 1660 through the portion of the shaft delivery lumen 1616 extending through the expandable distal end portion 1614 can cause the pleat portions of the plurality of pleats 1630 to move apart, thereby transforming the expandable distal end portion 1614 from the closed state to the open state. Advancing the introducer 1660 through the shaft delivery lumen 1616 can cause the plurality of pleats 1630 to unfold such that the expandable distal end portion 1614 can assume the open state.

In some instances, the introducer 1660 can be configured to be preloaded within the expandable access sheath 1600. For example, at least a portion of the introducer 1660 can be disposed within the sheath delivery lumen 1602 while at least a portion of the shaft 1610 is advanced along an anatomical pathway to a target position. In some instances, a portion of the introducer 1660 can be disposed distally of a distal end 1618 of the shaft 1610 while the shaft 1610 is advanced to its target position. A portion of the introducer 1660 can be extending through a distal opening 1620 at the distal end 1618 of the shaft 1610. At least a portion of the distal portion 1664 of the introducer 1660, such as a distal end 1668 of the introducer 1660 can initially be distal of the distal end 1618 of the shaft 1610. In some instances, the introducer 1660 can be subsequently advanced distally relative to the expandable access sheath 1600 to facilitate pushing apart the plurality of pleats 1630. For example, after the shaft 1610 is at a desired position within the anatomical pathway, the introducer 1660 can be subsequently advanced distally relative to the expandable access sheath 1600 to facilitate unfolding of the expandable distal end portion 1614 to the open state.

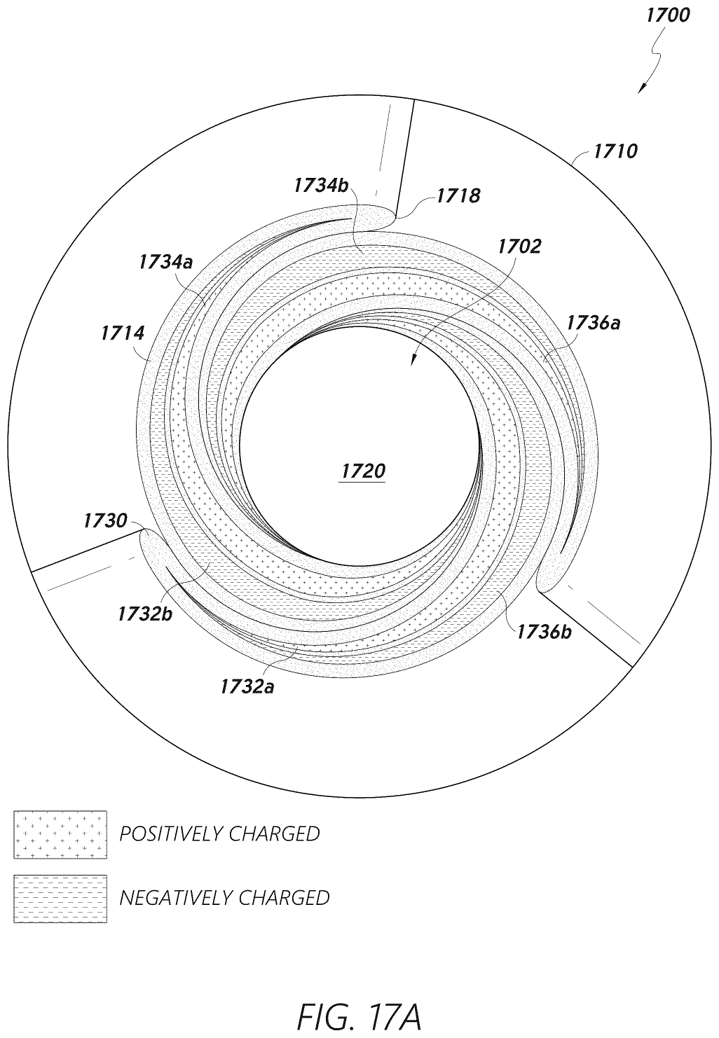

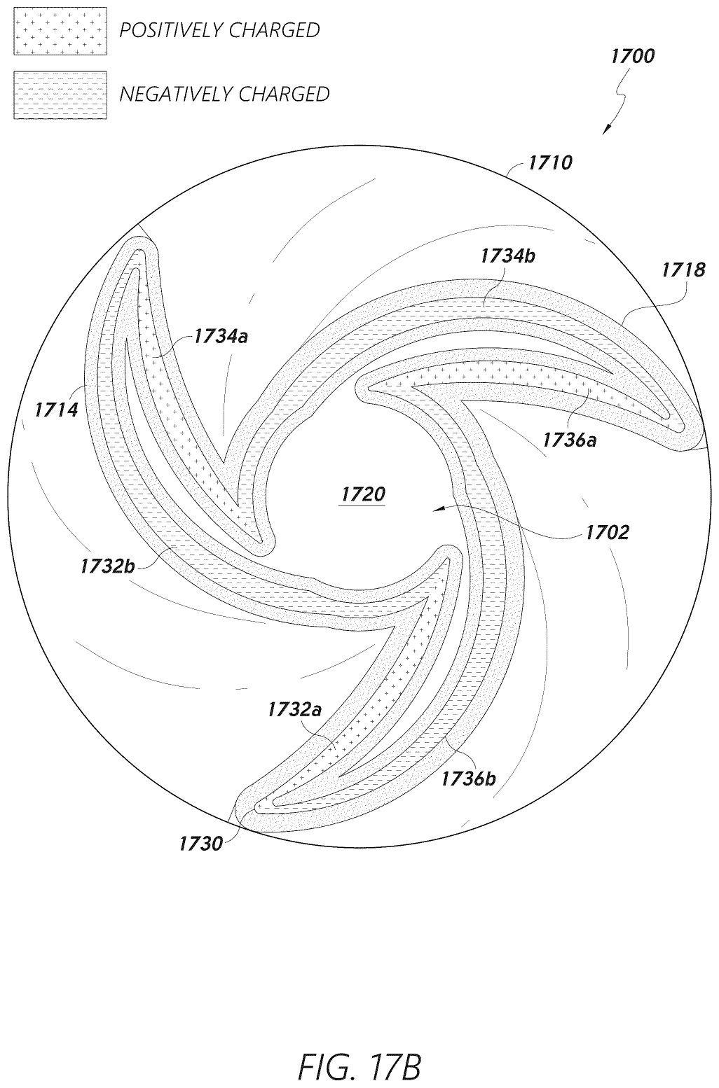

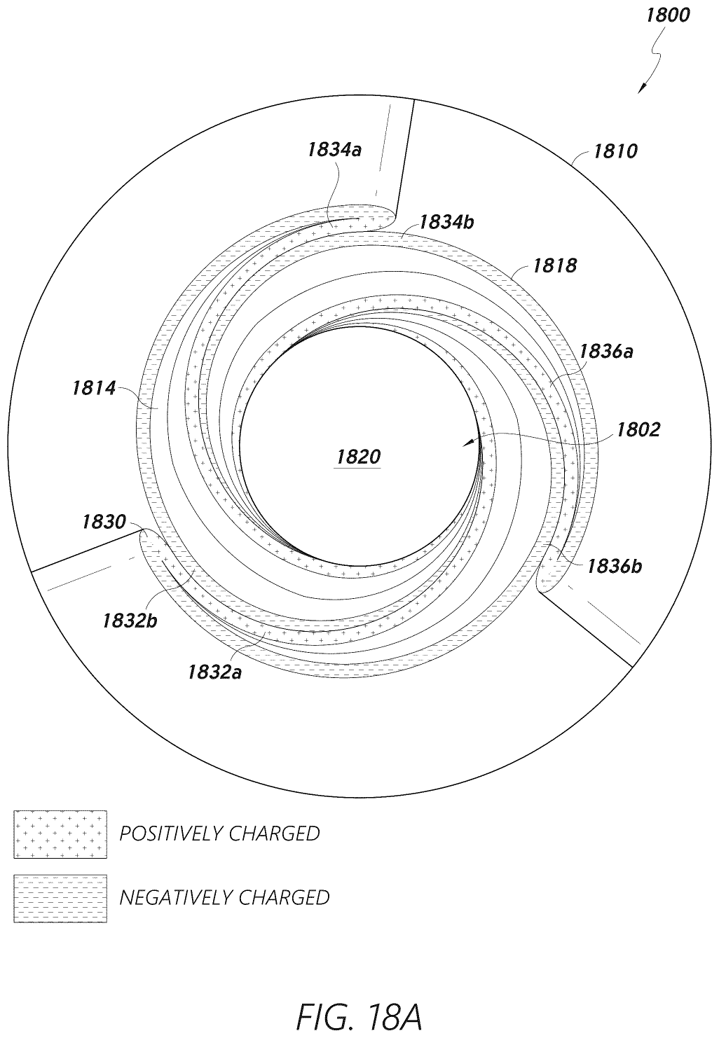

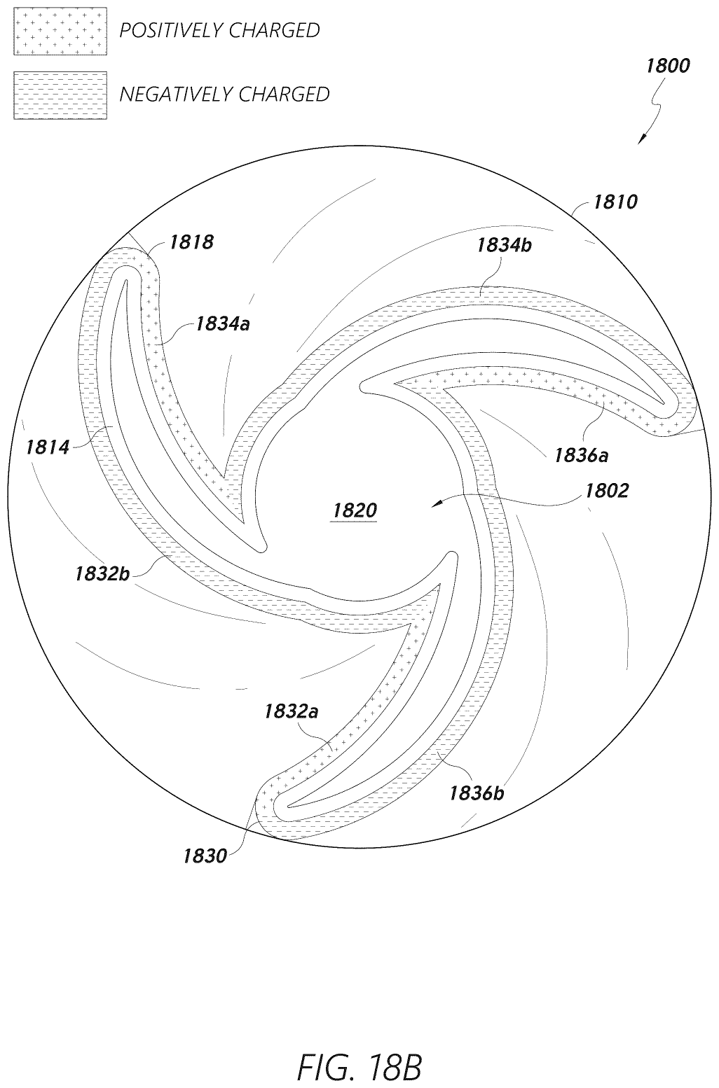

FIGS. 17A through 18B show examples of expandable access sheaths 1700, 1800 comprising a plurality of pleats, where a respective pair of the pleat portions of each pleat can be configured to be attracted to one another to maintain expandable distal end portions of the expandable access sheaths in a closed state. FIGS. 17A and 17B are distal end views of an example of an expandable access sheath 1700 comprising a shaft 1710 with an expandable distal end portion 1714 in a closed state and an open state, respectively. A distal opening 1720 at a distal end 1718 of the shaft 1710 is shown in FIGS. 17A and 17B. The expandable distal end portion 1714 is shown as comprising three pleats, each pleat comprising a pair of pleat portions configured to be oriented toward one another. It will be understood that the number of pleats is for illustrative purposes only and that more or fewer pleats can be applicable. A respective pleat portion of each pair of pleat portions can comprise magnetic material having opposite magnetic poles. In some instances, the respective pleat portion of each pair of pleat portions can comprise magnetic material, including magnetic particles and/or powder, having the opposite magnetic poles embedded therein. In some instances, a wall portion of the expandable distal end portion 1714 can comprise a plurality of layers, where the magnetic material can form one of the plurality of layers. For example, the magnetic material can form a layer between two other layers forming the wall portion.

Referring to FIG. 17A, the expandable distal end portion 1714 can comprise a first pleat 1732 comprising a second pair of pleat portions 1732a, 1732b, a second pleat 1734 comprising a second pair of pleat portions 1734a, 1734b, and a third pleat 1736 comprising a third pair of pleat portions 1736a, 1736b. Each of the pairs of pleat portions can be configured to be oriented toward one another while the expandable distal end portion 1714 is in the closed state. For example, the first pair of pleat portions 1732a, 1732b can be oriented toward one another. The second pair of pleat portions 1734a, 1734b can be oriented toward one another. The third pair of pleat portions 1736a, 1736b can be oriented toward one another. A pleat portion of each of the pairs of pleat portions can comprise magnetic material having an opposite magnetic pole as the other pleat portion in the pair. For example, the first pleat portions 1732a, 1734a, 1736a can comprise magnetic material having a positive pole, and the second pleat portions 1732b, 1734b, 1736b can comprise magnetic material having a negative pole. The magnetic material comprising opposite poles can be attracted to one another, facilitating attractions between the respective pleat portions.

FIG. 17B shows the expandable distal end portion 1714 in the open state. As described herein, at least a portion of a medical instrument, such as an introducer, can be advanced through a sheath delivery lumen 1702 of the expandable access sheath 1700 to cause the plurality of pleats 1730 to unfold. For example, while at least a portion of the introducer is disposed through the distal opening 1720, the expandable distal end portion 1714 can be in the open state. The pleat portions 1732a, 1732b, 1734a, 1734b, 1736a, 1736b are shown in further detail in FIG. 17B. As shown, respective pleat portions of each pair of pleat portions can comprise magnetic material having opposite poles to facilitate attraction of the pleat portions while the expandable distal end portion 1714 is in the closed state. Force exerted upon the pleats 1730, such as radial force exerted thereupon by the introducer as a portion of the introducer is advanced through the distal opening 1720 can overcome the attraction of the magnetic material such that the pleats 1730 can unfold.

FIGS. 18A and 18B are distal end views of another example of an expandable access sheath 1800 comprising an expandable distal end portion 1814 in a closed state and an open state, respectively. The expandable access sheath 1800 can comprise a shaft 1810 having the expandable distal end portion 1814. A distal opening 1820 at a distal end 1818 of the shaft 1810 is shown in FIGS. 18A and 18B. Although the expandable distal end portion 1814 is shown as comprising three pleats, it will be understood that the number of pleats is for illustrative purposes only and that more or fewer pleats can be applicable. A surface portion of a respective pleat portion of each pair of pleat portions can comprise opposite electrical charges. In some instances, the surface portion of the respective pleat portion comprises a plurality of positively charged electrical particles or a plurality of negatively charged electrical particles.

Referring to FIG. 18A, the expandable distal end portion 1814 can comprise a first pleat 1832 comprising a first pair of pleat portions 1832a, 1832b, a second pleat 1834 comprising a second pair of pleat portions 1834a, 1834b, and a third pleat 1836 comprising a third pair of pleat portions 1836a, 1836b. Each of the pairs of pleat portions can be configured to be oriented toward one another while the expandable distal end portion 1814 is in the closed state. For example, the first pair of pleat portions 1832a, 1832b can be oriented toward one another. The second pair of pleat portions 1834a, 1834b can be oriented toward one another. The third pair of pleat portions 1836a, 1836b can be oriented toward one another. A pleat portion of each of the pairs of pleat portions can comprise on a surface portion an opposite electrostatic charge as the other pleat portion in the pair. For example, the first pleat portions 1832a, 1834a, 1836a can comprise material having a positive electrical charge on a respective surface portion, and the second pleat portions 1832b, 1834b, 1836b can comprise material having a negative electrical charge on a respective surface portion. The material comprising opposite electrical charges can be attracted to one another, facilitating attractions between the respective pleat portions.

FIG. 18B shows the expandable distal end portion 1814 in the open state. As described herein, at least a portion of a medical instrument, such as an introducer, can be advanced through a sheath delivery lumen 1802 of the expandable access sheath 1800 to cause the plurality of pleats 1830 to unfold. For example, while at least a portion of the introducer is disposed through the distal opening 1820, the expandable distal end portion 1814 can be in the open state. The pleat portions 1832a, 1832b, 1834a, 1834b, 1836a, 1836b are shown in further detail in FIG. 18B. As shown, respective pleat portions of each pair of pleat portions can comprise oppositely charged electrostatic charges to facilitate attraction of the pleat portions while the expandable distal end portion 1814 is in the closed state. A pleat portion of each pair of the pleat portion can comprise on a surface portion electrostatic charge that is opposite that one the other pleat portion of the pair. Force exerted upon the pleats 1830, such as radial force exerted thereupon by the introducer as a portion of the introducer is advanced through the distal opening 1820 can overcome the attraction of the electrostatic charges such that the pleats 1830 can unfold.

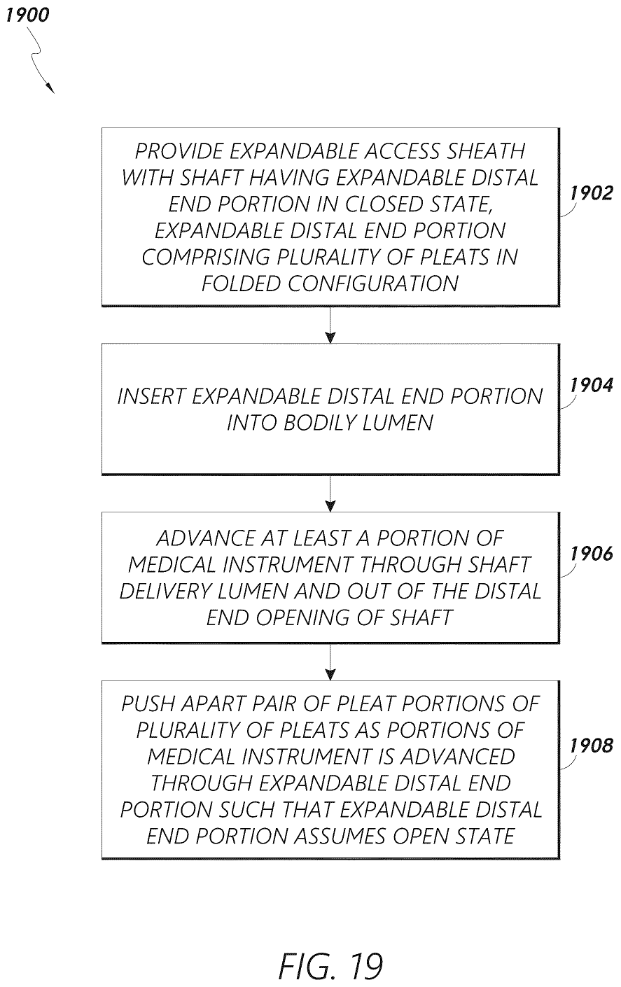

FIG. 19 is a process flow diagram of an example of a process 1900 for delivering a medical device to a target site using an expandable access sheath having one or more features of the expandable sheaths described with reference to FIGS. 16A to 18B. In block 1902, the process can involve providing an expandable access sheath. The expandable access sheath can comprise a shaft having an expandable distal end portion. The expandable distal end portion can be provided in a closed state. The shaft can have a distal end opening in fluid communication with a shaft delivery lumen extending therethrough. The expandable distal end portion can comprise a plurality of pleats in a folded configuration while the expandable distal end portion is in the closed state. Each of the plurality of pleats can comprise a pair of pleat portions configured to be oriented toward and attracted to one another while the plurality of pleats is in the folded configuration.

In block 1904, the process can involve inserting the expandable distal end portion into a bodily vessel and/or lumen. At least a portion of the shaft can be advanced along an anatomical pathway.

In block 1906, the process can involve advancing at least a portion of a medical instrument through the shaft delivery lumen and out of the distal end opening of the shaft. For example, at least a portion of an introducer can be advanced through the shaft delivery lumen and out of the distal end opening such that a distal portion of the introducer can be disposed distally of the distal end opening.

In block 1908, the process can involve pushing apart the pair of pleat portions of the plurality of pleats as a portion of the medical instrument is advanced through the expandable distal end portion such that the expandable distal end portion can assume an open state. The pair of pleat portions configured to be oriented toward and attracted to one another while the plurality of pleats is in the folded configuration can be pushed apart as the portion of the medical instrument is advanced through a corresponding portion of the shaft delivery lumen extending through the expandable distal end portion to allow the expandable distal end portion to assume the open state. Advancing the portion of the medical instrument through the portion of the shaft delivery lumen extending through the expandable distal end portion can result in pushing apart of the plurality of pairs of pleat portions of the plurality of pleats. For example, advancing a portion of an introducer through the shaft delivery lumen, including disposing a distal portion of the introducer distally of the distal end opening, can result in pushing apart the plurality of pleats. Causing the circumferential portion of the expandable distal end portion to assume an opened state can comprise causing the plurality of pleats of the expandable distal end to unfold.

In some instances, advancing at least a portion of the medical instrument through the shaft delivery lumen can comprise advancing at least a portion of a medical device through the delivery lumen of the shaft. The medical device can be carried on and/or within a delivery catheter. For example, at least a portion of a delivery catheter configured to receive the medical device can be advanced through the shaft delivery lumen. In some instances, the delivery catheter can be first advanced through the shaft delivery lumen. The medical device can subsequently be deployed from the delivery catheter. For example, at least a portion of the medical device can be advanced through the delivery catheter for deployment from the delivery catheter.

As described herein, in some instances, pleat portions can comprise magnetic material. For example, pushing apart the pair of pleat portions can comprise pushing apart a pair of pleat portions each comprising magnetic material embedded therein. In some instances, the pleat portions can comprise electrostatic charges. For example, pushing apart the pair of pleat portions can comprise pushing apart a pair of pleat portions each comprising a surface portion having electrical charges thereon.

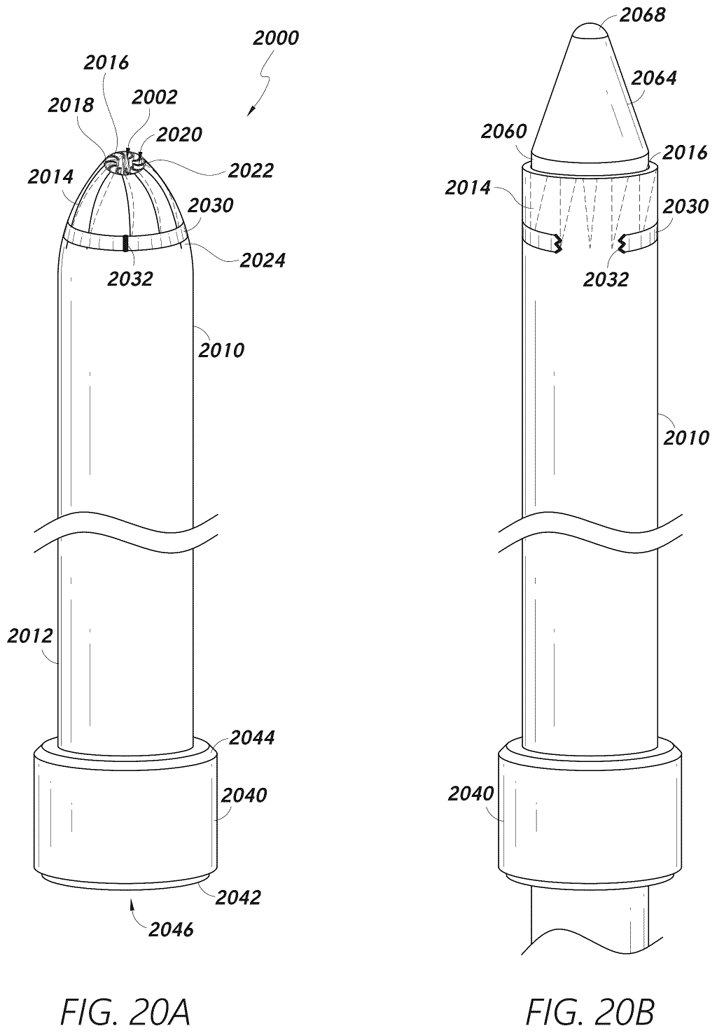

FIGS. 20A and 20B are perspective views of an example of an expandable access sheath 2000 that includes a shaft 2010 comprising an expandable distal end portion 2014 having a circumferential portion 2030 with a weakened joint 2032 associated therewith. FIG. 20A shows the expandable distal end portion 2014 in a closed state and FIG. 20B shows the expandable distal end portion 2014 in an open state. Referring to FIG. 20A, while the expandable distal end portion 2014 is in the closed state, the circumferential portion 2030 can be configured to be circumferentially disposed around a portion of the expandable distal end portion 2014. The circumferential portion 2030 can be configured to separate along the weakened joint 2032 to allow the expandable distal end portion 2014 to assume the open state, such as shown in FIG. 20B. In some instances, the expandable distal end portion 2014 comprises a plurality of pleats. The pleats can be configured to be in a folded configuration while the expandable distal end portion 2014 is in the closed state. The pleats can be configured to be in an unfolded configuration while the expandable distal end portion 2014 is in the open state.

The expandable access sheath 2000 can comprise a sheath delivery lumen 2002 extending therethrough and configured to allow advancement of a medical device and/or delivery catheter configured to carry the medical device through the expandable access sheath 2000. The expandable access sheath 2000 can comprise a shaft 2010 and a hub 2040. The shaft 2010 can comprise a proximal portion 2012 associated with the hub 2040, such as a distal end 2044 of the hub 2040. The proximal portion 2012 can be coupled to the distal end 2044 of the hub 2040. The shaft can extend distally from the hub 2040, including from the distal end 2044 of the hub 2040. The shaft 2010 can comprise a shaft delivery lumen 2016 and the hub 2040 can comprise a hub delivery lumen 2046 extending from a proximal end 2042 to a distal end 2044 of the hub 2040. The sheath delivery lumen 2002 can comprise the shaft delivery lumen 2016 and the hub delivery lumen 2046. The shaft delivery lumen 2016 can be in communication with and/or be coaxial with the hub delivery lumen 2046.

The expandable distal end portion 2014 can comprise circumferential portion 2030 with the weakened joint 2032. The weakened joint 2032 can be integrated with and/or coupled to the circumferential portion 2030. In some instances, the circumferential portion 2030 and/or the weakened joint 2032 can be integrated with the expandable distal end portion 2014, including being embedded within and/or as a part of a wall portion of the expandable distal end portion 2014. For example, the circumferential portion 2030 can form a part of the side wall of the expandable distal end portion 2014. Alternatively, the circumferential portion 2030 can be disposed over an externally oriented surface of the expandable distal end portion 2014. For example, the circumferential portion 2030 can be disposed over and coupled to an externally oriented surface of a wall portion of the expandable distal end portion 2014.

The circumferential portion 2030 can be disposed around a circumferential portion proximal of a distal end 2018 of the shaft 2010. The position of the circumferential portion 2030 on the expandable distal end portion 2014 can be at or around a medial point along a longitudinal dimension of the expandable distal end portion 2014. In some instances, the longitudinal dimension can be parallel or substantially parallel to a longitudinal axis of the shaft 2010. In some instances, the longitudinal dimension can be a longitudinal dimension along an externally oriented surface of the expandable distal end portion 2014. For example, the longitudinal dimension along the externally oriented surface of the expandable distal end portion 2014 can extend from a proximal end 2024 to a distal end 2022 of the expandable distal end portion 2014. The position of the circumferential portion 2030 can be selected to facilitate maintaining the expandable distal end portion 2014 in the closed state.

Although the circumferential portion 2030 is shown as extending along only a portion of the expandable distal end portion 2014, it will be understood that the circumferential portion 2030 can have a length the same or similar to that of the expandable distal end portion 2014. In some instances, the circumferential portion 2030 can comprise an elastic material. In some instances, the circumferential portion 2030 can be elastic. For example, the circumferential portion 2030 with the weakened joint 2032 can be an elastic ring comprising a weakened joint. The elastic ring can be configured to stretch, such as radially stretch, and separate along the weakened joint to allow the expandable distal end portion 2014 to assume the open state. In some instances, an elastic circumferential portion can extend along an entire length of the expandable distal end portion 2014. For example, an elastic circumferential portion can comprise a ring configuration, extending along only a portion of the expandable distal end portion, while the expandable distal end portion is in the closed state, such as shown in FIG. 20A. Alternatively, the expandable distal end portion 2014 can be entirely or substantially entirely the elastic circumferential portion with the weakened joint. For example, the elastic circumferential portion can extend circumferentially around the expandable distal end portion 2014, and from the proximal end 2024 to the distal end 2022 of the expandable distal end portion 2014.

In some instances, the circumferential portion 2030 can comprise an inelastic material. In some instances, the circumferential portion 2030 can comprise a metallic material configured to separate along the weakened joint 2032. In some instances, the circumferential portion 2030 with the weakened joint 2032 can comprise a metal ring comprising a weakened joint. The metal ring can be configured to separate along the weakened joint to allow the expandable distal end portion 2014 to assume the open state.

In some instances, a medical instrument can be advanced through the expandable distal end portion 2014 to facilitate transformation of the expandable distal end portion 2014 from the closed state to the open state. Referring to FIG. 20B, at least a portion of an introducer 2060 can be advanced through the sheath delivery lumen 2002 to cause the expandable distal end portion 2014 to expand and/or unfold and assume the open state. For example, a distal portion 2064 of the introducer 2060 can be advanced through a corresponding portion of the shaft delivery lumen 2016 extending through the expandable distal end portion 2014 such that at least a portion of the introducer 2060 is disposed distally of a distal end 2018 of the shaft 2010, including the distal portion 2064 of the introducer 2060. The circumferential portion 2030 can stretch and/or deform as the portion of the introducer 2060 is advanced through the expandable distal end portion 2014 and out of a distal end opening 2020 at the distal end 2018 of the shaft 2010. The circumferential portion 2030 can break and/or separate along the weakened joint 2032 as the introducer 2060 is advanced through the corresponding portion of the shaft delivery lumen 2016.

As described herein, in some instances, the circumferential portion 2030 can comprise metal. Advancing at least a portion of an introducer 2060 through the shaft delivery lumen 2016 can cause an expandable distal portion comprising the metallic material to deform. Deformation of the metallic circumferential portion can lead to separation and/or breakage of the metallic circumferential portion along the weakened joint. The circumferential portion 2030 can be a metal ring member comprising a weakened joint that is associated with the expandable distal end portion 2014. In some instances, the metal ring can be an integral part of the expandable distal end portion 2014. In some instances, the metal ring can be coupled to an external surface portion of the expandable distal end portion 2014. Radial force exerted upon the expandable distal end portion 2014 by portions of the introducer 2060 as the introducer 2060 is advanced through the expandable distal end portion 2014 can result in deformation of the metal ring member and subsequent breakage and/or separation along the weakened joint of the metal ring. An elastic circumferential portion can be stretched as the introducer 2060 is advanced through the corresponding portion of the shaft delivery lumen 2016 extending through the expandable distal end portion 2014. Stretching of the elastic circumferential portion can lead to its breakage and/or separation along the weakened joint.

In some instances, the introducer 2060 can be configured to be preloaded within the expandable access sheath 2000. For example, at least a portion of the introducer 2060 can be disposed within the sheath delivery lumen 2002 while at least a portion of the shaft 2010 is advanced along an anatomical pathway to a target position. In some instances, a portion of the introducer 2060 can be disposed distally of a distal end 2018 of the shaft 2010 while the shaft 2010 is advanced to its target position, including at least a portion of the distal portion 2064 of the introducer 2060, such as a distal end 2068 of the introducer 2060.

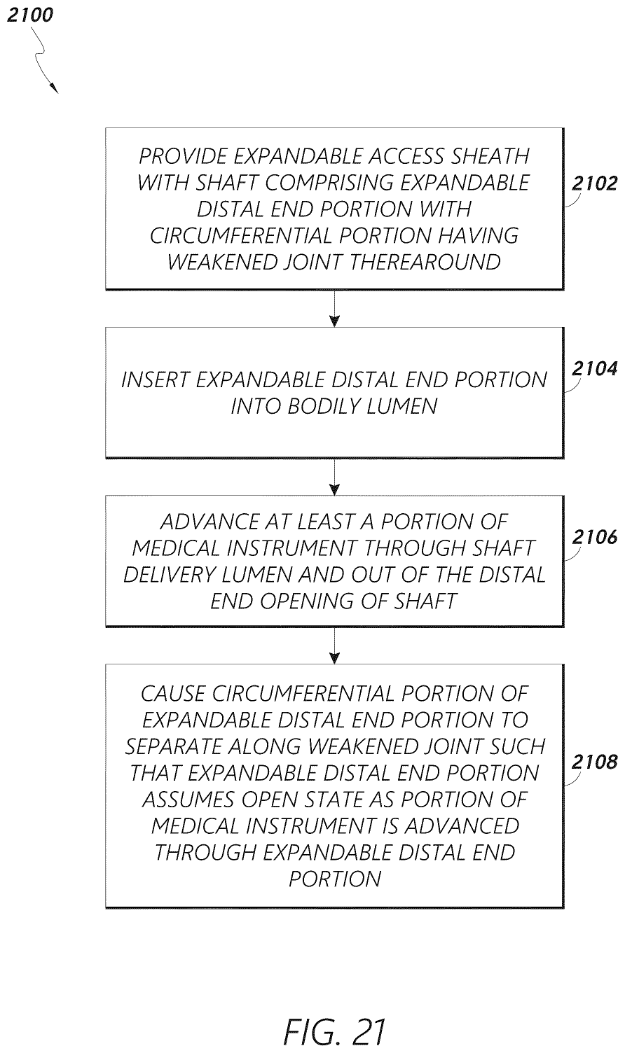

FIG. 21 is a process flow diagram of an example of a process 2100 for delivering a medical device to a target site using an expandable access sheath having one or more features of the expandable sheath described with reference to FIGS. 20A and 20B. In block 2102, the process can involve providing an expandable access sheath. The expandable access sheath can comprise a shaft having an expandable distal end portion. The expandable distal end portion can be provided in a closed state. The shaft can have a distal end opening in fluid communication with a shaft delivery lumen extending through the shaft delivery lumen. The expandable distal end portion can have a circumferential portion disposed around the expandable distal end portion and comprising a weakened joint.

In block 2104, the process can involve inserting the expandable distal end portion into a bodily vessel and/or lumen. At least a portion of the shaft can be advanced along an anatomical pathway.

In block 2106, the process can involve advancing at least a portion of a medical instrument through the shaft delivery lumen and out of the distal end opening of the shaft. In some instances, the medical instrument can comprise an introducer. For example, at least a portion of an introducer can be advanced through the shaft delivery lumen and out of the distal end opening such that a distal portion of the introducer can be disposed distally of the distal end opening.

In block 2108, the process can involve causing the circumferential portion of the expandable distal end portion to separate along the weakened joint such that the expandable distal end portion can assume an opened state as a portion of the medical instrument is advanced through the expandable distal end portion. For example, pushing the portion of the medical instrument through a corresponding portion of the shaft delivery lumen extending through the expandable distal end portion can cause the circumferential portion to stretch radially and separate along the weakened joint. Separation of the circumferential portion along the weakened joint can allow the expandable distal end portion to expand and/or unfold.

In some instances, the circumferential portion can comprise a metal. For example, the circumferential portion can comprise a metallic material. In some instances, causing the circumferential portion of the expandable distal end portion to separate along the weakened joint can comprise causing a metallic circumferential portion to separate along the weakened joint. In some instances, the circumferential portion can comprise an elastic material. For example, the circumferential portion can comprise an elastic circumferential portion. In some instances, causing the circumferential portion of the expandable distal end portion to separate along the weakened joint can comprise causing an elastic circumferential portion to separate along the weakened joint.

In some instances, the expandable distal end portion can be in a folded configuration while in the closed state. Causing the expandable distal end portion to assume an opened state can comprise causing the expandable distal end portion to unfold. In some instances, the expandable distal end portion can comprise a plurality of pleats configured to be in a folded configuration while the expandable distal end portion is in the closed state. For example, causing the circumferential portion of the expandable distal end portion to assume an opened state can comprise causing the expandable distal end to unfold a plurality of pleats.

In some instances, advancing at least a portion of the medical instrument through the shaft delivery lumen can comprise advancing at least a portion of a medical device through the delivery lumen of the shaft. The medical device can be carried on and/or within a delivery catheter. For example, at least a portion of a delivery catheter configured to receive the medical device can be advanced through the shaft delivery lumen. In some instances, the delivery catheter can be first advanced through the shaft delivery lumen. The medical device can subsequently be deployed from the delivery catheter. For example, at least a portion of the medical device can be advanced through the delivery catheter to be deployed from the delivery catheter.

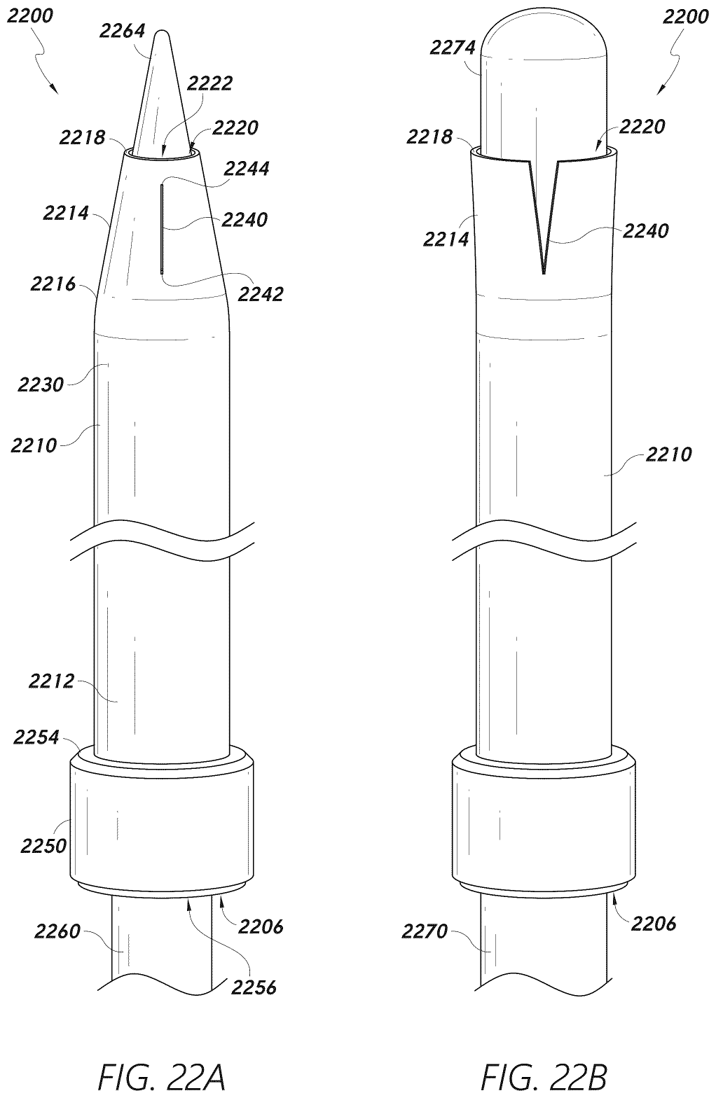

FIGS. 22A through 25 relate to various examples of expandable access sheaths, each comprising a shaft with an expandable distal end portion that, while the expandable distal end portion is in a closed state, the expandable distal end portion includes one or more weakened portions. The expandable distal end portion can separate along the one or more weakened portions such that the expandable distal end portion can transform from the closed state to an open state. The weakened portion can be formed in a sidewall of the expandable distal end portion. The weakened portion can facilitate transformation of the expandable distal end portion to the open state while providing an expandable distal end portion with desired column strength and/or able to remain in the closed state for navigation through anatomical pathways. It will be understood that one or more features described with reference to the expandable access sheaths of FIGS. 22A through 24B can be combined into one expandable access sheath.

FIGS. 22A and 22B are side views of an example of an expandable access sheath 2200 comprising a shaft 2210 with an expandable distal end portion 2214 that includes a weakened portion 2240 in a sidewall 2230 of the shaft 2210. FIG. 22A is a side view of the expandable access sheath 2200 while the expandable distal end portion 2214 is in a closed state. FIG. 22B is a side view of the expandable access sheath 2200 while the expandable distal end portion 2214 is in an open state. Referring to FIG. 22A, while the expandable distal end portion 2214 is in the closed state, the sidewall 2230 of the expandable distal end portion 2214 can be configured to have the weakened portion 2240. The expandable distal end portion 2214 can be expanded and/or stretched such that it separates along the weakened portion 2240 to transform the expandable distal end portion 2214 from the closed state to the open state. For example, the sidewall 2230 of the expandable distal end portion 2214 can rip, tear and/or split along the weakened portion 2240.

At least a portion of the weakened portion 2240 can have an orientation that is at an angle relative to a distal end 2218 of the shaft 2210. The distal end 2218 of the shaft 2210 can be a distal end of the expandable distal end portion 2214. In some instances, the weakened portion 2240 can be parallel or substantially parallel to a longitudinal axis of the shaft 2210. In some instances, the weakened portion 2240 can have an orientation that is perpendicular or substantially perpendicular to a portion of the distal end 2218. For example, the weakened portion 2240 can be perpendicular or substantially perpendicular to a portion of the distal end 2218 adjacent to and/or closest to the weakened portion 2240.

The weakened portion 2240 can extend along a portion of the expandable distal end portion 2214. In some instances, the weakened portion 2240 does not extend to the distal end 2218 of the shaft 2210. For example, a distal end 2244 of the weakened portion 2240 can be at a distance from and/or be spaced from the distal end 2218 of the shaft 2210. In some instances, the distal end 2244 of the weakened portion 2240 can be at least about 1 millimeter (mm) from the distal end 2218 of the shaft 2210. Having the distal end 2244 of the weakened portion 2240 not be at the distal end 2218 of the shaft 2210 can avoid or reduce premature opening of the expandable distal end portion 2214, while facilitating opening of the expandable distal end portion 2214 after the shaft 2210 is desirably positioned. In some instances, the distal end 2244 of the weakened portion 2240 can be between about 1 millimeter (mm) to about 2 millimeter (mm), including about 1 millimeter (mm) to about 1.5 millimeter (mm), from the distal end 2218 of the shaft 2210. A proximal end 2242 of the weakened portion 2240 can be at or distal of a proximal end 2216 of the expandable distal end portion 2214. In some instances, a length of the weakened portion can be selected to provide an expandable distal end portion with desired column strength and/or ability to maintain the closed state for navigation through anatomical pathways, while facilitating transformation of the expandable distal end portion to the open state.

As shown in FIG. 22A, the weakened portion 2240 can comprise a pre-formed slit through a portion of the sidewall 2230 of the shaft 2210. At least a portion of the pre-formed slit can be oriented at an angle relative to the distal end 2218 of the shaft 2210, including a portion of the distal end 2218 closest to the pre-formed slit. In some instances, the weakened portion 2240 can be a pre-formed slit through a portion of the sidewall 2230. At least a portion of the pre-formed slit can extend along a line, including a linear or substantially linear line, having an orientation that is at an angle relative to the distal end 2218 of the shaft 2210. In some instances, the pre-formed slit can extend along a line that is parallel or substantially parallel to the longitudinal axis of the shaft 2210. A distal end of the pre-formed slit can be about 1 millimeter (mm) to about 2 millimeter (mm), including about 1 millimeter (mm) to about 1.5 millimeter (mm), from the distal end 2218 of the shaft 2210.

In some instances, the expandable distal end portion 2214 can comprise a polymeric material. In some instances, the sidewall 2230 of the expandable distal end portion 2214 can be formed using a polymeric material. For example, while in the closed state, the expandable distal end portion 2214 can assume a tapered profile. A lateral dimension, such as a diameter, of the expandable distal end portion 2214 can decrease along a dimension of the shaft 2210 extending distally along the longitudinal axis toward the distal end 2218 of the shaft 2210. The lateral dimension can be perpendicular or substantially perpendicular to the longitudinal axis of the shaft 2210. The polymeric sidewall can assume the tapered profile, for example forming the tapered expandable distal end portion 2214 while the expandable distal end portion 2214 is in the closed state. The weakened portion 2240, such as the pre-formed slit, can extend through the polymeric material. The polymeric sidewall of the expandable distal end portion 2214 can separate along the weakened portion 2240 to allow the expandable distal end portion 2214 to assume the open state.

In some instances, the expandable distal end portion 2214 can comprise polyethylene, including low density polyethylene (LDPE) and/or ultrahigh molecular weight polyethylene (UHMWPE). In some instances, the expandable distal end portion 2214 can comprise both low density polyethylene (LDPE) and ultrahigh molecular weight polyethylene (UHMWPE), including one or more layers of low-density polyethylene (LDPE) and/or ultrahigh molecular weight polyethylene (UHMWPE). For example, the weakened portion 2240 can be formed through a plurality of polymer layers of the sidewall 2230, including a plurality of layers of low-density polyethylene (LDPE) and/or ultrahigh molecular weight polyethylene (UHMWPE).

In the alternative or in combination, a weakened portion can comprise an indentation along a sidewall of a shaft that does not extend through the entire thickness of the sidewall. In the alternative or in combination, a weakened portion can comprise a perforated portion, including for example, a perforated line. For example, at least a portion of an indentation and/or perforated portion can extend along a line, including a linear or substantially linear line, at an angle relative to a distal end of the shaft. The indentation and/or perforated portion can extend along a line that is parallel or substantially parallel to a longitudinal axis of the shaft. In some instances, an expandable distal end portion can comprise a plurality of weakened portions. In some instances, the plurality of weakened portions can be evenly distributed circumferentially around a sidewall of the expandable distal end portion. For example, a plurality of pre-formed slits, indentations and/or perforated portions can be at respective positions on the sidewall forming the expandable distal end portion, including at respective positions evenly distributed around the circumference of the expandable distal end portion.

The expandable access sheath 2200 can comprise a hub 2250. The shaft 2210 can extend distally from the hub 2250, and can be associated with a distal end 2254 of the hub 2250. For example, a proximal portion 2212 of the shaft 2210 can be coupled to the distal end 2254 of the hub 2250. The hub 2250 can comprise a hub delivery lumen 2256 extending through the hub 2250. The hub delivery lumen 2256 can be in fluid communication with and/or be coaxial with a shaft delivery lumen 2222 extending through the shaft 2210. A sheath delivery lumen 2206 extending through the expandable access sheath 2200 can comprise the hub delivery lumen 2256 and the shaft delivery lumen 2222. The sheath delivery lumen 2206 can be configured to allow advancement therethrough of a medical device and/or delivery catheter configured to carry the medical device.

In some instances, a medical instrument, such as an introducer can be preloaded through the expandable access sheath 2200 to facilitate navigation of the expandable access sheath 2200 through tortuous anatomical pathways. FIG. 22A shows a first introducer 2260 disposed through the hub delivery lumen 2256 and the shaft delivery lumen 2222 while the expandable distal end portion is in the closed state. A distal portion 2264 of the first introducer 2260 can be disposed through a distal end opening 2220 in fluid communication with the shaft delivery lumen 2222 and be disposed distally of the distal end 2218 of the shaft 2210 to facilitate advancement of the expandable access sheath 2200 to a target location.

In some instances, at least a portion of a medical instrument can be advanced through the expandable distal end portion 2214 to cause the expandable distal end portion 2214 to separate along the weakened portion 2240. FIG. 22B shows a second introducer 2270 being advanced through a respective portion of the shaft delivery lumen 2222 extending through the expandable distal end portion 2214, for example positioning a distal portion 2274 of the second introducer 2270 distally of the distal end 2218 of the shaft 2210. Advancement of at least a portion of the second introducer 2270 through the shaft delivery lumen 2222 extending through the expandable distal end portion 2214 can cause the expandable distal end portion 2214 to expand and/or stretch. For example, the second introducer 2270 can engage with the sidewall 2230 such that the sidewall 2230 of the expandable distal end portion 2214 can rip, tear and/or split along the weakened portion 2240.

Alternatively, a second introducer is not used and a first introducer used for navigating the shaft 2210 to the target location can comprise a dilation feature configured to cause the expandable distal end portion 2214 to separate along the weakened portion 2240. For example, after the expandable access sheath 2200 is desirably positioned, the first introducer can be advanced relative to the expandable access sheath 2200 to the sidewall of the expandable distal end portion 2214 can rip, tear and/or split along the weakened portion 2240.

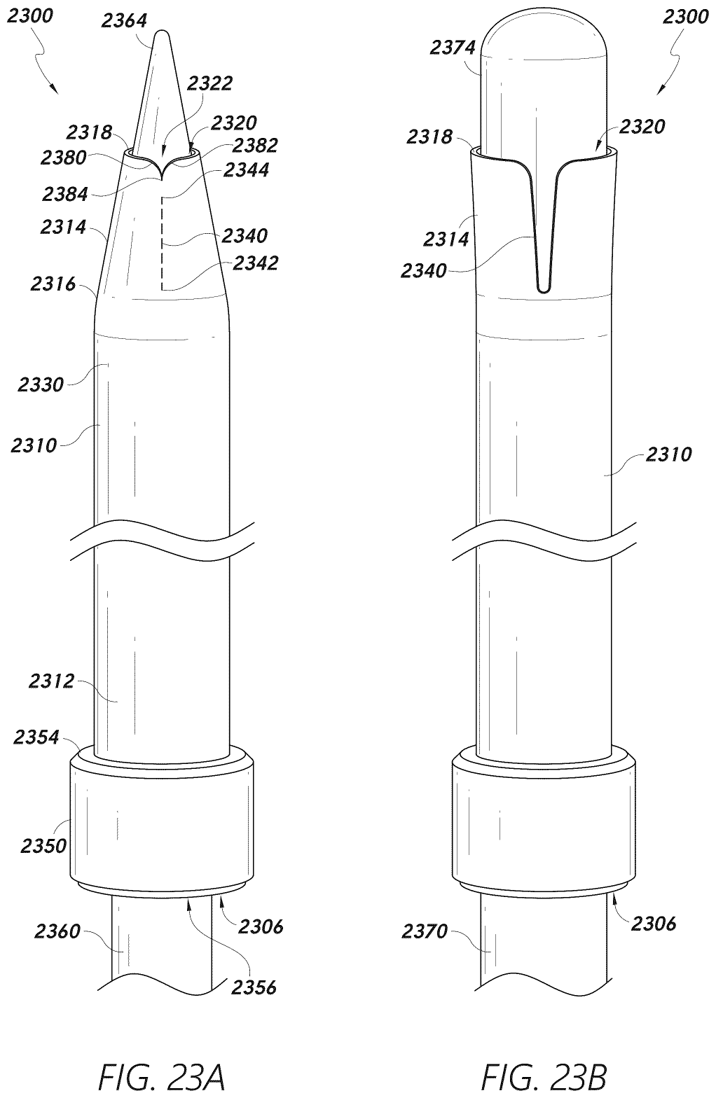

FIGS. 23A and 23B are side views of another example of an expandable access sheath 2300 comprising a shaft 2310 with an expandable distal end portion 2314 that includes a weakened portion 2340. A distal end 2318 of the shaft 2310 can form a plurality of convex curvatures. For example, at least a portion of a distal end 2318 of the shaft 2310 can form a plurality of segments of an ellipse. FIG. 23A shows the expandable distal end portion 2314 in a closed state, and FIG. 23B shows the expandable distal end portion 2314 in an open state. The weakened portion 2340 can be between two adjacent segments of ellipses. The weakened portion 2340 can be formed in a sidewall 2330 and have an orientation that is at an angle relative to the distal end 2318 of the shaft 2310, including being parallel or substantially parallel to a longitudinal axis of the shaft 2310. Portions of the distal end 2318 on two sides of the weakened portion 2340 can curve proximally toward the weakened portion 2340. For example, the sidewall 2230 at the distal end 2318 can form a first segment of an ellipse 2380 and a second segment of an ellipse 2382. The weakened portion 2340 can be between the first segment of the ellipse 2380 and the second segment of the ellipse 2382. For example, a distal end 2344 of the weakened portion 2340 can be a distance from and/or spaced from a portion of the distal end 2318 between the first segment of the ellipse 2380 and the second segment of the ellipse 2382. In some instances, the first segment of the ellipse 2380 and the second segment of the ellipse 2382 can meet at an intermediate portion 2384 of the distal end 2318. The weakened portion 2340 can be perpendicular or substantially perpendicular to the intermediate portion 2384. The weakened portion 2340 can extend proximally from its distal end 2344 along a direction parallel or substantially parallel to the longitudinal axis of the shaft 2310.

In some instances, the weakened portion 2340 does not extend to the distal end 2318 of the shaft 2310. For example, a distal end 2344 of the weakened portion 2340 can be at a distance from and/or be spaced from the distal end 2318 of the shaft 2310, including from the intermediate portion 2384. In some instances, the distal end 2344 of the weakened portion 2340 can be at least about 1 millimeter (mm) from the distal end 2318 of the shaft 2310, including between about 1 millimeter (mm) to about 2millimeter (mm), including about 1 millimeter (mm) to about 1.5 millimeter (mm), from the distal end 2318 of the shaft 2310, such as from the intermediate portion 2384 of the distal end 2318. A proximal end 2342 of the weakened portion 2340 can be at or distal of a proximal end 2316 of the expandable distal end portion 2314.

In some instances, the weakened portion can be a perforated portion, including a perforated line formed a portion of the sidewall 2330 of the shaft 2310. At least a portion of the perforated portion can be oriented at an angle relative to the distal end 2318 of the shaft 2310, including a portion of the distal end 2318 closest to the perforated portion. In some instances, the weakened portion 2340 can be a perforated portion, including a perforated line, through a portion of the sidewall 2330. At least a portion of the perforated portion can extend along a line, including a linear or substantially linear line, having an orientation that is at an angle relative to the distal end 2318 of the shaft 2310. In some instances, the perforated portion can extend along a line that is parallel or substantially parallel to the longitudinal axis of the shaft 2310. A distal end of the perforated portion can be about 1 millimeter (mm) to about 2 millimeter (mm), including about 1 millimeter (mm) to about 1.5 millimeter (mm), from the distal end 2318 of the shaft 2310.

In the alternative or in combination, a weakened portion can comprise an indentation along the sidewall of a shaft that does not extend through the entire thickness of the sidewall and/or a pre-formed slit. The indentation and/or pre-formed slit can be between adjacent segments of ellipses at a distal end of the shaft. For example, at least a portion of an indentation and/or pre-formed slit can extend along a line, including a linear or substantially linear line, at an angle relative to a distal end of the shaft and between two adjacent segments of ellipses. The indentation and/or pre-formed slit can extend along a line that is parallel or substantially parallel to a longitudinal axis of the shaft.

In some instances, the distal end 2318 can form a plurality of segments of circles. For example, the first segment of the ellipse 2380 can be a first segment of a circle and the second segment of the ellipse 2382 can be a second segment of a circle. The weakened portion 2340 can be between the two adjacent segments of circles. The distal end 2344 of the weakened portion 2340 can be a distance from and/or spaced from a portion of the distal end 2318, such as the intermediate portion 2384, between the first segment of the circle and the second segment of the circle.

Although FIGS. 23A and 23B show the shaft 2310 comprising one weakened portion, in some instances, a shaft can comprise an expandable distal end portion with a plurality of weakened portions. The plurality of weakened portions can be distributed, including evenly distributed, around a circumference of the expandable distal end portion. Respective portions of the distal end on two sides of each of the weakened portions can curve proximally toward each of the weakened portions. In some instances, the distal end can have scalloped shape while the shaft is in a closed state. In some instances, the distal end can form a plurality of segments of an ellipse, including a plurality of segments of circles, including a plurality of semi-ellipses and/or semi-circles. For example, each of a plurality of pre-formed slits, indentations and/or perforated portions can be at respective positions on the sidewall forming the expandable distal end portion between adjacent segments of ellipses.

Referring to FIG. 23B, the expandable distal end portion 2314 can be expanded and/or stretched such that it separates along the weakened portion 2340 to transform the expandable distal end portion 2314 from the closed state to the open state. For example, the sidewall 2330 of the expandable distal end portion 2314 can rip, tear and/or split along the weakened portion 2340. The curvatures of the distal end 2318 can provide an atraumatic distal end after the expandable distal end portion 2314 separates along the weakened portion 2340 to assume the open state.

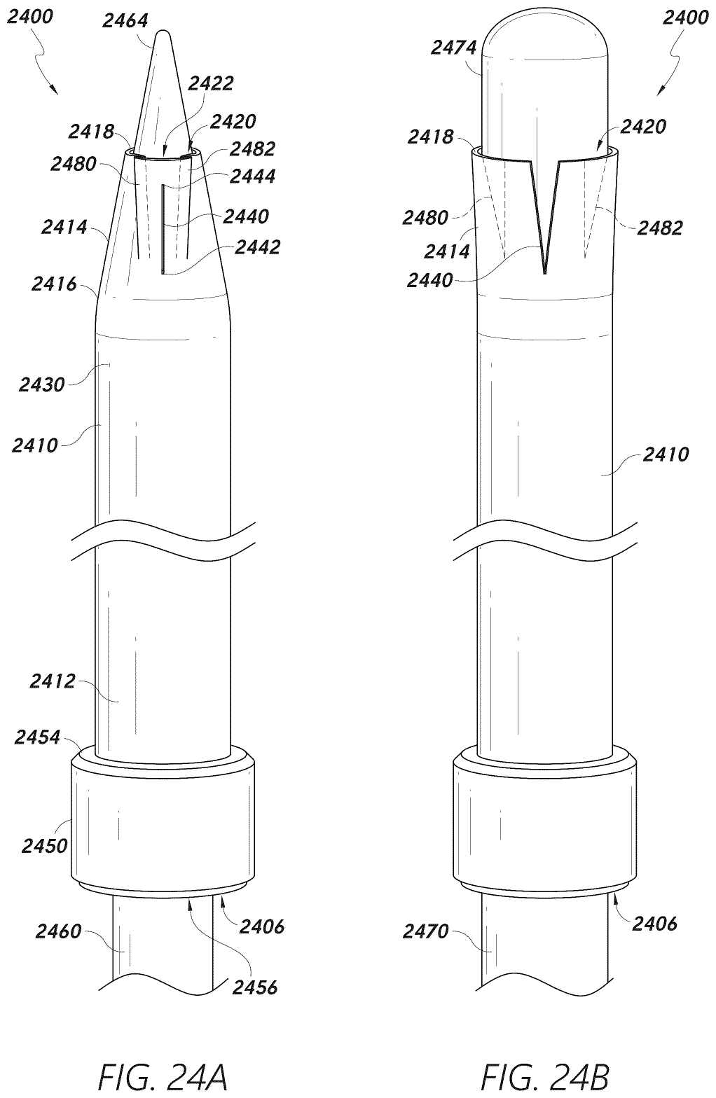

FIGS. 24A and 24B are side views of another example of an expandable access sheath 2400 comprising a shaft 2410 with an expandable distal end portion 2414 that includes a weakened portion 2440. The expandable distal end portion 2414 can comprise a plurality of pleats, such as a first pleat 2480 and a second pleat 2482, configured to be in a folded configuration while the expandable distal end portion 2414 is in a closed state, and in an unfolded configuration while the expandable distal end portion 2414 is in an open state. FIG. 24A shows the expandable distal end portion 2414 in a closed state, and FIG. 24B shows the expandable distal end portion 2414 in an open state. Referring to FIG. 24A, the weakened portion 2440 can be on a portion of the expandable distal end portion 2414 that is not part of the pleats while the expandable distal end portion 2414 is in the closed state. For example, the weakened portion 2440 can be on an unfolded portion of a sidewall of the shaft 2410 while the expandable distal end portion 2414 is in the closed state.

The weakened portion 2440 can be formed in the sidewall between the first pleat 2480 and the second pleat 2482. The weakened portion 2440 can have an orientation that is at an angle relative to the distal end 2418 of the shaft 2410, including being parallel or substantially parallel to a longitudinal axis of the shaft 2410. A distal end 2444 of the weakened portion 2440 can be at a distance and/or spaced from the distal end 2418 of the shaft 2410. In some instances, the distal end 2444 of the weakened portion 2440 can be at least about 1 millimeter (mm) from the distal end 2418 of the shaft 2410, including between about 1 millimeter (mm) to about 2 millimeter (mm), including about 1 millimeter (mm) to about 1.5 millimeter (mm), from the distal end 2418 of the shaft 2410. A proximal end 2442 of the weakened portion 2440 can be at or distal of a proximal end 2416 of the expandable distal end portion 2414.

In some instances, the weakened portion 2440 can comprise a pre-formed slit through a portion of the sidewall 2430 of the shaft 2410. At least a portion of the pre-formed slit can be oriented at an angle relative to the distal end 2418 of the shaft 2410, including a portion of the distal end 2418 closest to the pre-formed slit. In some instances, the weakened portion 2440 can be a pre-formed slit through a portion of the sidewall 2430. At least a portion of the pre-formed slit can extend along a line, including a linear or substantially linear line, having an orientation that is at an angle relative to the distal end 2418 of the shaft 2410 and/or being parallel or substantially parallel to the longitudinal axis of the shaft 2410. A distal end of the pre-formed slit can be about 1millimeter (mm) to about 2 millimeter (mm), including about 1 millimeter (mm) to about 1.5 millimeter (mm), from the distal end 2418 of the shaft 2410.

Referring to FIG. 24B, the expandable distal end portion 2414 can be expanded and/or stretched such that the plurality of pleats 2480, 2482 unfold and the sidewall 2430 of the expandable distal end portion 2414 separates along the weakened portion 2440 to transform the expandable distal end portion 2414 from the closed state to the open state. The sidewall 2430 of the expandable distal end portion 2414 can rip, tear and/or split along the weakened portion 2440.

In the alternative or in combination, a weakened portion can comprise an indentation along a sidewall of a shaft that does not extend through the entire thickness of the sidewall, including an indentation that extends a long a line, such as a linear or substantially linear line, at an angle relative to a distal end of the shaft. In the alternative or in combination, a weakened portion can comprise a perforated portion, including for example, a perforated line, such as a linear or substantially linear line, at an angle relative to a distal end of the shaft. The indentation and/or perforated portion can comprise at least a portion that extends along a line that is parallel or substantially parallel to a longitudinal axis of the shaft. In some instances, an expandable distal end portion can comprise a plurality of weakened portions. In some instances, the plurality of weakened portions can be at respective positions around the circumference of the expandable distal end portion that are not folded while the expandable distal end portion is closed. For example, the plurality of weakened portions can be at respective positions that are not part of a fold and/or a pleat. The plurality of weakened portions can be evenly distributed circumferentially around a sidewall of the expandable distal end portion and be on portions of the sidewall that are between adjacent pleats. For example, a plurality of pre-formed slits, indentations and/or perforated portions can be at respective positions on the sidewall forming the expandable distal end portion, including at respective positions evenly distributed around the circumference of the expandable distal end portion, that are between adjacent pleats.

In some instances, the expandable access sheaths 2300, 2400 described with reference to FIGS. 23A-24B can have one or more other features of the expandable access sheath 2200 described with reference to FIGS. 22A and 22B. For example, the expandable access sheaths 2300, 2400 can each comprise a hub 2350, 2450. Each of the shafts 2310, 2410 can extend distally from the respective hub 2350, 2450. A proximal portion 2312, 2412 of the shaft 2310, 2410 be coupled to a distal end 2354, 2454 of the hub 2350, 2450. The hubs 2350, 2450 can each comprise a hub delivery lumen 2356, 2456 extending through the hub 2350, 2450, and in fluid communication with and/or be coaxial with a shaft delivery lumen 2322, 2422 extending through the shaft 2310, 2410. A sheath delivery lumen 2306, 2406 extending through the expandable access sheath 2300, 2400 can comprise the hub delivery lumen 2356, 2456 and the shaft delivery lumen 2322, 2422. FIGS. 23A and 24A show a medical instrument, such as an introducer, preloaded through the sheath delivery lumen 2306, 2406, and a distal portion 2364, 2464 of the medical instrument 2360, 2460 being disposed through a distal end opening 2320, 2420 and disposed distally of the distal end 2318, 2418 of the shaft 2310, 2410 to facilitate navigation of the expandable access sheaths 2300, 2400 through tortuous anatomical pathways. At least a portion of a medical instrument can be advanced through the expandable distal end portions 2314, 2414 to cause the expandable distal end portions 2314, 2414 to separate along the weakened portion 2340, 2440. FIGS. 23B and 24B show that an introducer different from that used for navigating the expandable access sheaths 2300, 2400 through the anatomical pathway can be used to cause the expandable distal end portions 2314, 2414 to assume the open state. For example, a second introducer 2370, 2470 can be advanced through a respective portion of the shaft delivery lumen 2322, 2422 extending through the expandable distal end portion 2314, 2414, to position a distal portion 2374, 2474 of the second introducer 2370, 2470 distally of the shaft 2310, 2410. The second introducer 2370, 2470 can engage with the sidewall 2330, 2430 such that the sidewall 2330, 2430 of the expandable distal end portion 2314 can rip, tear and/or split along the weakened portion 2340, 2440. Alternatively, a different introducer is not used for expanding and/or opening the expandable distal end portions 2314, 2414. The introducer used to navigate the expandable access sheaths 2300, 2400 through the anatomical pathway can comprise a dilation feature configured to cause the expandable distal end portions 2314, 2414 to separate along the weakened portion 2340, 2440. For example, after the expandable access sheaths 2300, 2400 are desirably positioned, the introducer can be advanced relative to the expandable access sheaths 2300, 2400 to the sidewall of the expandable distal end portions 2314, 2414 can rip, tear and/or split along the weakened portion 2340, 2440.

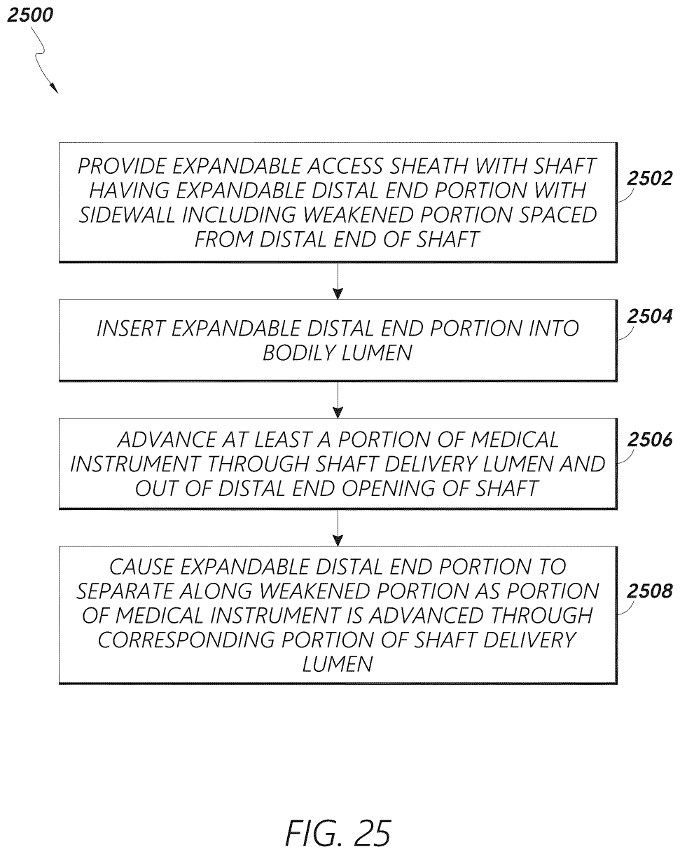

FIG. 25 is a process flow diagram illustrating an example of a medical device delivery process 2500 using one or more expandable access sheaths described herein, including for example, the expandable access sheaths 2200, 2300, 2400 described with reference to FIGS. 22A, 22B, 23A, 23B, 24A and 24B. In block 2502, the process can involve providing an expandable access sheath with a shaft having expandable distal end portion with a sidewall that includes a weakened portion while the expandable distal end portion is in a closed state. A distal end of the weakened portion can be at a distance and/or spaced from a distal end of the shaft. At least a portion of the weakened portion can have an orientation that is at an angle relative to the distal end of the shaft, including for example being perpendicular or substantially perpendicular to the distal end of the shaft. In block 2504, the process can involve inserting the expandable distal end portion into a bodily lumen. In block 2506, the process can involve advancing at least a portion of a medical instrument through a shaft delivery lumen of the shaft and out of a distal end opening of the shaft. In block 2508, the process can involve causing the expandable distal end portion to separate along the weakened portion of the sidewall as a portion of the medical instrument is advanced through a corresponding portion of the shaft delivery lumen to allow the expandable distal end portion to assume an open state.

In some instances, causing the expandable distal end portion to separate along the weakened portion can comprise causing the expandable distal end portion to separate along a weakened line, including a weakened line having an orientation that is parallel or substantially parallel to a longitudinal axis of the shaft. In some instances, causing the expandable distal end portion to separate along the weakened portion can comprise causing a perforated portion, including a perforated line, on the sidewall of the expandable distal end portion to separate. In some instances, causing the expandable distal end portion to separate along the weakened portion can comprise causing a preformed slit on the sidewall of the expandable distal end portion to separate. In some instances, causing the expandable distal end portion to separate along the weakened portion can comprise causing the expandable distal end portion to separate along a plurality of weakened portions, including a plurality of weakened portions evenly distributed around a circumference of the expandable distal end portion.

In some instances, at least a portion of the distal end of the shaft can form a plurality of segments of ellipses. Causing the expandable distal end portion to separate along the weakened portion can comprise causing the expandable distal end portion to separate along a weakened portion between two adjacent segments of ellipses, including two adjacent segments of circles.

In some instances, the expandable distal end portion can comprise a plurality of pleats. The weakened portion can be on an unfolded sidewall portion of the expandable distal end portion while the expandable distal end portion is in the closed state. The process can involve causing the plurality of pleats of the expandable distal end portion to unfold as a portion of the medical instrument is advanced through a corresponding portion of the shaft delivery lumen. In some instances, causing the distal end portion to separate along the weakened portion can comprise causing the expandable distal end portion to separate along the weakened portion on the unfolded sidewall portion.

In some instances, advancing at least a portion of a medical instrument through a shaft delivery lumen comprises pushing a portion of the medical instrument against the sidewall of the expandable distal end portion to cause separation of the sidewall along the weakened portion. In some instances, pushing the portion of the medical instrument against the sidewall of the expandable distal end portion can comprise engaging a distal end portion of the medical instrument with the sidewall of the shaft to cause separation of the sidewall along the weakened portion.

### Additional Description of Examples

Provided below is a list of examples, each of which may include aspects of any of the other examples disclosed herein. Furthermore, aspects of any example described above may be implemented in any of the numbered examples provided below.

Example 1: An introducer comprising an elongate shaft configured to be received within an access sheath, the elongate shaft including a distal end and a proximal end, and a fixed-form expansion feature disposed on the elongate shaft between the distal end and the proximal end, the fixed-form expansion feature including a larger outer diameter than the distal end of the elongate shaft and configured to dilate at least a distal end portion of the access sheath when the elongate shaft is translated distally within the access sheath.

Example 2: The introducer of any example herein, in particular example 1, wherein the elongate shaft includes a tapered portion between the distal end of the elongate shaft and the fixed-form expansion feature, the tapered portion tapering toward the distal end of the elongate shaft.

Example 3: The introducer of any example herein, in particular example 1 or 2, further comprising: an introducer hub coupled to the elongate shaft and configured to selectively engage with a sheath hub, the introducer hub being configured to disengage from the sheath hub when the introducer is translated distally.

Example 4: The introducer of any example herein, in particular example 3, wherein the introducer hub is configured to disengage from the sheath hub when the introducer is translated distally by more than a threshold distance, the threshold distance being associated with at least one of a position of the fixed-form expansion feature on the elongate shaft or a length of the access sheath.

Example 5: The introducer of any of any example herein, in particular examples 1-4, wherein an outer diameter of the fixed-form expansion feature is larger than an inner diameter of a distal end of the access sheath in a pre-dilated state.

Example 6: The introducer of any example herein, in particular examples 1-5, wherein the elongate shaft includes a lumen to receive a guidewire.

Example 7: An access system comprising an expandable access sheath including a lumen, and an introducer configured to axially move within the lumen of the expandable access sheath, the introducer including a fixed expansion feature on a distal section of the introducer, the fixed expansion feature including a larger outer diameter than an inner diameter of at least a portion of the expandable access sheath in a non-expanded state, the introducer being configured to radially expand the expandable access sheath when the introducer is translated within the expandable access sheath.

Example 8: The access system of any example herein, in particular example 7, further comprising a sheath hub coupled to the expandable access sheath, and an introducer hub coupled to the introducer and configured to selectively engage with the sheath hub, the introducer hub being configured to disengage from the sheath hub when the introducer is moved distally.

Example 9: The access system of any example herein, in particular examples 7-8, further comprising a locking mechanism coupled to at least one of the expandable access sheath or the introducer, the locking mechanism being configured to prevent removal of the introducer from the expandable access sheath until the fixed expansion feature is moved distally beyond a distal end of the expandable access sheath.

Example 10: The access system of any example herein, in particular examples 7-9, wherein the expandable access sheath is tapered such that a distal end of the expandable access sheath has a smaller diameter than a proximal end of the expandable access sheath.

Example 11: The access system of any example herein, in particular examples 7-10, wherein the expandable access sheath is permanently expandable.

Example 12: The access system of any example herein, in particular examples 7-11, wherein the expandable access sheath is elastically expandable.

Example 13: The access system of any example herein, in particular examples 7-12, wherein the introducer includes a tapered portion between a distal end of the introducer and the fixed expansion feature, the tapered portion tapering toward the distal end of the introducer.

Example 14: A method of accessing an anatomical feature, the method comprising advancing an access sheath into a patient anatomy, the access sheath including a lumen, distally translating a first introducer through the lumen to cause an expansion feature on the first introducer to radially expand at least a distal end of the access sheath, the expansion feature including a fixed form, removing the first introducer from the lumen of the access sheath, and advancing a medical instrument through the lumen of the access sheath.

Example 15: The method of any example herein, in particular example 14, wherein the advancing the access sheath into the patient anatomy includes inserting a second introducer into the access sheath such that at least a distal end portion of the second introducer extends beyond the distal end of the access sheath and advancing the access sheath with the second introducer disposed therein into the patient anatomy.

Example 16: The method of any example herein, in particular example 15, further comprising upon advancing the access sheath with the second introducer disposed therein into the patient anatomy, and removing the second introducer from the access sheath.

Example 17: The method of any example herein, in particular examples 14-16, wherein the distally translating the first introducer includes distally translating the first introducer by at least a threshold distance to disable a locking mechanism and allow the first introducer to be removed from the access sheath.

Example 18: The method of any example herein, in particular examples 14-17, wherein the advancing the access sheath into the patient anatomy includes inserting the first introducer into the lumen in the access sheath such that a distal end of the first introducer extends beyond the distal end of the access sheath and advancing the access sheath with the first introducer disposed therein into the patient anatomy.

Example 19: The method of any example herein, in particular examples 14-18, wherein the first introducer includes a tapered portion between a distal end of the first introducer and the expansion feature, the tapered portion tapering toward the distal end of the first introducer.

Example 20: The method of any example herein, in particular examples 14-19, wherein the access sheath is permanently expandable.

Example 21: The method of any example herein, in particular examples 14-20, wherein the access sheath is temporarily expandable.

The above method(s) can be performed on a living animal or on a simulation, such as on a cadaver, cadaver heart, anthropomorphic ghost, simulator (e.g., with body parts, heart, tissue, etc. being simulated).

Example 22: A medical access system comprising an expandable access sheath, the expandable access sheath comprising a hub, and a shaft extending distally from the hub, the shaft having an expandable distal end portion configured to be in an open state and a closed state and a side shaft opening on the expandable distal end portion in fluid communication with a shaft delivery lumen extending through the shaft. The medical access system can include a cord comprising a first cord tail portion configured to pass through the side shaft opening, a second cord tail portion configured to pass through the side shaft opening, and a medial cord portion between the first and second cord tail portions, the medial cord portion being configured to be disposed around a circumferential portion of the expandable distal end portion when the expandable distal end portion is in the closed state.

Example 23: The system of any example herein, in particular example 22, wherein the side shaft opening comprises a first side shaft opening and a second side shaft opening, and the first and the second cord tail portions are configured to extend through a respective one of the first and second side shaft openings and extend proximally along a portion of the shaft delivery lumen.

Example 24: The system of any example herein, in particular example 22 or 23, wherein the cord is configured to be removed from the expandable access sheath to allow the expandable distal end portion to assume the open state.

Example 25: The system of any example herein, in particular example 22, further comprising an introducer, the introducer comprising a central lumen extending through at least a portion of the introducer, and a side introducer opening on a distal portion thereof, the side introducer opening in fluid communication with the central lumen, and while the expandable distal end portion is in the closed state, at least a portion of the introducer being configured to be slidably received within the shaft delivery lumen, and the side introducer opening being configured to be within the shaft delivery lumen, and the first and second cord tail portions being configured to extend through the side shaft opening and the side introducer opening and along at least a portion of the central lumen of the introducer.

Example 26: The system of any example herein, in particular example 25, wherein, while the expandable distal end portion is in the closed state, the side shaft opening is configured to be aligned with the side introducer opening.

Example 27: The system of any example herein, in particular example 25 or 26, wherein the side shaft opening comprises a first side shaft opening and a second side shaft opening, the side introducer opening comprises a first side introducer opening and a second side introducer opening, and the first and the second cord tail portions are configured to extend through a respective one of the first and second side shaft openings and a respective one of the first and second side introducer openings.

Example 28: The system of any example herein, in particular examples 25-27, wherein the introducer is configured to be translated proximally relative to the expandable access sheath to withdraw the introducer from within the shaft delivery lumen, and the first and second side introducer openings are configured to slide proximally along at least a portion of the first and second cord tail portions extending through the shaft delivery lumen as the introducer is translated proximally.

Example 29: The system of any example herein, in particular examples 25-27, wherein the cord is configured to be removed from around the expandable access sheath and the central lumen of the introducer while the at least a portion of the introducer is received within the shaft delivery lumen to allow the expandable distal end portion to assume the open state.

Example 30: The system of any example herein, in particular examples 22-29, wherein a portion of at least one of the first tail cord portion and the second tail cord portion is configured to be positioned around a portion of the circumferential portion of the expandable distal end portion to allow the expandable distal end portion to transform to the open state from the closed state.

Example 31: The system of any example herein, in particular examples 22-30, wherein a proximal portion of the first tail cord portion and a proximal portion of the second tail cord portion are configured to be secured to the hub of the expandable access sheath to maintain the expandable distal end portion in the closed state or the open state.

Example 32: An expandable access sheath comprising a hub, and a shaft extending distally from the hub, the shaft comprising an expandable distal end portion and the expandable distal end portion comprising a plurality of pleats. The plurality of pleats can be configured to be in a folded configuration while the expandable distal end portion is in a closed state, each of the plurality of pleats comprising a pair of pleat portions configured to be oriented toward one another in the folded configuration, the pair of pleat portions being configured to be attracted to one another to maintain the folded configuration, and an unfolded configuration while the expandable distal end portion is in an open state.

Example 33: The sheath of any example herein, in particular example 32, wherein a respective pleat portion of each pair of pleat portions comprises magnetic material having opposite magnetic poles.

Example 34: The sheath of any example herein, in particular example 33, wherein the respective pleat portion of each pair of pleat portions comprises magnetic material having the opposite magnetic poles embedded therein.

Example 35: The sheath of any example herein, in particular example 32, wherein a surface portion of a respective pleat portion of each pair of pleat portions comprises opposite electrostatic charges.

Example 36: The sheath of any example herein, in particular example 35, wherein the surface portion of the respective pleat portion comprises a plurality of positively charged electrical particles or a plurality of negatively charged electrical particles.

Example 37: The sheath of any example herein, in particular examples 32-36, wherein the expandable access sheath is configured to receive an introducer and advancing a distal portion of the introducer through the expandable distal end portion is configured to push apart the plurality of pleats to allow the expandable distal end portion to assume an open state.

Example 38: An expandable access sheath comprising a hub, and a shaft extending distally from the hub, the shaft comprising an expandable distal end portion and the expandable distal end portion comprising a circumferential portion having a weakened joint. The circumferential portion, while the expandable distal end portion is in a closed state, can be configured to be circumferentially disposed around a portion of the expandable distal end portion, and configured to separate along the weakened joint to allow the expandable distal end portion to assume an open state.

Example 39: The sheath of any example herein, in particular example 38, wherein the circumferential portion comprises a metal ring comprising the weakened joint, and the metal ring is configured to separate along the weakened joint to allow the expandable distal end portion to assume the open state.

Example 40: The sheath of any example herein, in particular example 38, wherein the circumferential portion comprises an elastic ring comprising a weakened joint, and the elastic ring is configured to stretch and separate along the weakened joint to allow the expandable distal end portion to assume the open state.

Example 41: The sheath of any example herein, in particular examples 38-40, wherein the expandable distal end portion comprises a plurality of pleats and wherein the plurality of pleats are configured to be in a folded configuration while the expandable distal end portion is in the closed state, and the plurality of pleats are configured to be in an unfolded configuration while the expandable distal end portion is in the open state.

Example 42: A method of delivering a medical device, the method comprising providing a medical access system. The medical access system can comprise an expandable access sheath comprising a shaft extending distally from a hub, the shaft having an expandable distal end portion, and a first and a second side shaft opening on the expandable distal end portion in fluid communication with a shaft delivery lumen extending through the shaft. The medical access system can comprise a cord comprising a first cord tail portion, a second cord tail portion, and a medial cord portion between the first and second cord tail portions. The method can include inserting the expandable distal end portion into a bodily lumen while the expandable distal end portion is in a closed state, the medial cord portion being disposed around a circumferential portion of the expandable distal end portion, and the first and second cord tail portions extending through a respective one of the first and second side shaft openings; and reducing the tension on the cord to position a portion of at least one of the first and second cord tail portions over the circumferential portion of the expandable distal end portion to allow the expandable distal end portion to assume an open state.

Example 43: The method of any example herein, in particular example 42, further comprising one of passing the medial cord portion and then the second tail cord portion through the first and second side shaft openings and the delivery lumen as the first tail cord portion is translated proximally to remove the cord and allow the expandable distal end portion to assume the open state; and passing the circumferential cord portion and then the first tail cord portion through the first and second side shaft openings and the shaft delivery lumen as the second tail cord portion is translated proximally to remove the cord and allow the expandable distal end portion to assume the open state.

Example 44: The method of any example herein, in particular example 42, further comprising providing an introducer at least partially received in the shaft delivery lumen, the introducer comprising a central lumen extending through at least a portion thereof, and a first and a second side introducer opening in fluid communication with the central lumen and disposed within the shaft delivery lumen, and the first and second cord tail portions extending through a respective one of the first and second side shaft openings and a respective one of the first and second side introducer openings.

Example 45: The method of any example herein, in particular example 44, further comprising translating the introducer proximally relative to the expandable access sheath to withdraw the introducer from within the shaft delivery lumen, the first and second side introducer openings sliding proximally along at least a portion of the first and second cord tail portions.

Example 46: The method of any example herein, in particular example 44, further comprising one of passing the medial cord portion and then the second tail cord portion through the first and second side introducer openings and the central lumen as the first tail cord portion is translated proximally to remove the cord and allow the expandable distal end portion to assume the open state; and passing the medial cord portion and then the first tail cord portion through the first and second side introducer openings and the central lumen as the second tail cord portion is translated proximally to remove the cord and allow the expandable distal end portion to assume the open state.

Example 47: The method of any example herein, in particular examples 42-46, further comprising securing a proximal portion of the first tail cord portion and a proximal portion of the second tail cord portion to the hub of the expandable access sheath to maintain the expandable distal end portion in the closed state or the open state.

Example 48: The method of any example herein, in particular examples 42-47, further comprising tensioning the cord to transform the expandable distal end portion of the shaft from the open state to the closed state, and translating the expandable distal end portion proximally to withdraw the shaft from the bodily lumen.

The above method(s) can be performed on a living animal or on a simulation, such as on a cadaver, cadaver heart, anthropomorphic ghost, simulator (e.g., with body parts, heart, tissue, etc. being simulated).

Example 49: A method of delivering a medical device, the method comprising providing an expandable access sheath, the expandable access sheath comprising a shaft having an expandable distal end portion in a closed state, and a distal end opening in fluid communication with a shaft delivery lumen extending therethrough, the expandable distal end portion comprising a plurality of pleats in a folded configuration while in the closed state, each of the plurality of pleats comprising a pair of pleat portions configured to be oriented toward and attracted to one another while the pleats are in the folded configuration. The method can include inserting the expandable distal end portion into a bodily lumen, advancing at least a portion of a medical instrument through the shaft delivery lumen of the shaft and out of the distal end opening, and pushing apart the pair of pleat portions configured to be oriented toward and attracted to one another while the pleats are in the folded configuration as a portion of the medical instrument is advanced through a corresponding portion of the delivery lumen to allow the expandable distal end portion to assume an open state.

Example 50: The method of any example herein, in particular example 49, wherein pushing apart the pair of pleat portions comprises pushing apart a pair of pleat portions each comprising magnetic material embedded therein.

Example 51: The method of any example herein, in particular example 49, wherein pushing apart the pair of pleat portions comprises pushing apart a pair of pleat portions each comprising a surface portion having electrostatic charges thereon.

Example 52: The method of any example herein, in particular examples 49-51, wherein advancing the at least a portion of the medical instrument comprises advancing the at least a portion of an introducer through the shaft delivery lumen.

Example 53: The method of any example herein, in particular examples 49-51, wherein advancing the at least a portion of the medical instrument comprises advancing at least a portion of a medical device through the shaft delivery lumen.

Example 54: The method of any example herein, in particular examples 49-53, wherein causing the circumferential portion of the expandable distal end portion to assume an opened state comprises causing the plurality of pleats of the expandable distal end to unfold.

The above method(s) can be performed on a living animal or on a simulation, such as on a cadaver, cadaver heart, anthropomorphic ghost, simulator (e.g., with body parts, heart, tissue, etc. being simulated).

Example 55: A method of delivering a medical device, the method comprising providing an expandable access sheath, the expandable access sheath comprising a shaft having an expandable distal end portion in a closed state, and a distal end opening in fluid communication with a shaft delivery lumen extending therethrough, and a circumferential portion comprising a weakened joint, the circumferential portion being disposed around a portion of the expandable distal end portion. The method can include inserting the expandable distal end portion into a bodily lumen, advancing at least a portion of a medical instrument through the delivery lumen of the shaft and out of the distal end opening, and causing the circumferential portion of the expandable distal end portion to separate along the weakened joint and assume an opened state as a portion of the medical instrument is advanced through a corresponding portion of the delivery lumen extending through the expandable distal end portion.

Example 56: The method of any example herein, in particular example 55, wherein causing the circumferential portion of the expandable distal end portion to separate along the weakened joint comprises causing a metallic circumferential portion to separate along the weakened joint.

Example 57: The method of any example herein, in particular example 55, wherein causing the circumferential portion of the expandable distal end portion to separate along the weakened joint comprises causing an elastic circumferential portion to separate along the weakened joint.

Example 58: The method of any example herein, in particular examples 55-57, wherein advancing the at least a portion of the medical instrument comprises advancing the at least a portion of an introducer through the shaft delivery lumen.

Example 59: The method of any example herein, in particular examples 55-57, wherein advancing the at least a portion of the medical instrument comprises advancing at least a portion of a medical device through the shaft delivery lumen.

Example 60: The method of any example herein, in particular examples 55-59, wherein causing the circumferential portion of the expandable distal end portion to assume an opened state comprises causing the expandable distal end to unfold a plurality of pleats.

The above method(s) can be performed on a living animal or on a simulation, such as on a cadaver, cadaver heart, anthropomorphic ghost, simulator (e.g., with body parts, heart, tissue, etc. being simulated).

Example 61: An expandable access sheath comprising a hub and a shaft extending distally from the hub, the shaft comprising an expandable distal end portion configurable in a closed state in which a sidewall of the expandable distal end portion includes a weakened portion having a distal end spaced from a distal end of the shaft and at least a portion of the weakened portion having an orientation at an angle relative to the distal end of the shaft. The expandable distal end portion can be configured to separate along the weakened portion to provide the expandable distal end portion in an open state.

Example 62: The sheath of any example herein, in particular example 61, wherein the weakened portion comprises at least a portion having an orientation parallel to a longitudinal axis of the shaft.

Example 63: The sheath of any example herein, in particular example 61 or 62, wherein the weakened portion comprises a weakened line.

Example 64: The sheath of any example herein, in particular examples 61 to 63, wherein the weakened portion comprises a perforated portion of the sidewall of the expandable distal end portion.

Example 65: The sheath of any example herein, in particular example 64, wherein the perforated portion is a perforated line.

Example 66: The sheath of any example herein, in particular examples 61 to 65, wherein the weakened portion comprises a preformed slit formed through the sidewall of the expandable distal end portion.

Example 67: The sheath of any example herein, in particular examples 61 to 66, wherein the expandable distal end portion comprises a plurality of weakened portions evenly distributed circumferentially around the sidewall of the expandable distal end portion.

Example 68: The sheath of any example herein, in particular examples 61 to 67, wherein the distal end of the weakened portion is about 1 millimeter (mm) to about 1.5 millimeters (mm) from the distal end of the shaft.

Example 69: The sheath of any example herein, in particular examples 61 to 68, wherein at least a portion of the distal end of the shaft forms a plurality of segments of ellipses, and the weakened portion is between two adjacent segments of ellipses.

Example 70: The sheath of any example herein, in particular example 69, wherein the distal end of the shaft forms a plurality of segments of circles, and the weakened portion is between two adjacent segments of circles.

Example 71: The sheath of any example herein, in particular examples 61 to 70, wherein the expandable distal end portion comprises a plurality of pleats configured to be in a folded configuration while the expandable distal end portion is in the closed state, and wherein the weakened portion is on an unfolded sidewall portion while the expandable distal end portion is in the closed state.

Example 72: A method of delivering a medical device, the method comprising providing an expandable access sheath having a shaft, the shaft comprising an expandable distal end portion configurable in a closed state in which a sidewall of the expandable distal end portion includes a weakened portion having a distal end spaced from a distal end of the shaft and at least a portion of the weakened portion having an orientation at an angle relative to the distal end of the shaft. The method can include inserting the expandable distal end portion into a bodily lumen, advancing at least a portion of a medical instrument through a shaft delivery lumen of the shaft and out of a distal end opening of the shaft, and causing the expandable distal end portion to separate along the weakened portion of the sidewall as a portion of the medical instrument is advanced through a corresponding portion of the shaft delivery lumen to allow the expandable distal end portion to assume an open state.

Example 73: The method of any example herein, in particular example 72, wherein causing the expandable distal end portion to separate along the weakened portion comprises causing a perforated line on the sidewall of the expandable distal end portion to separate.

Example 74: The method of any example herein, in particular example 72 or 73, wherein causing the expandable distal end portion to separate along the weakened portion comprises causing a preformed slit on the sidewall of the expandable distal end portion to separate.

Example 75: The method of any example herein, in particular examples 72 to 74, wherein causing the expandable distal end portion to separate along the weakened portion comprises causing the expandable distal end portion to separate along a plurality of weakened portions evenly distributed around a circumference of the expandable distal end portion.

Example 76: The method of any example herein, in particular examples 72 to 75, wherein at least a portion of the distal end of the shaft forms a plurality of segments of ellipses, and the causing the expandable distal end portion to separate along the weakened portion comprises causing the expandable distal end portion to separate along a weakened portion between two adjacent segments of ellipses.

Example 77: The method of any example herein, in particular examples 72 to 76, further comprising causing a plurality of pleats of the expandable distal end portion to unfold as a portion of the medical instrument is advanced through a corresponding portion of the shaft delivery lumen.

Example 78: The method of any example herein, in particular example 77, wherein causing the distal end portion to separate along the weakened portion comprises causing the expandable distal end portion to separate along a weakened portion on an unfolded sidewall portion of the expandable distal end portion while the expandable distal end portion is in the closed state.

Example 79: The method of any example herein, in particular examples 72 to 78, wherein advancing at least a portion of a medical instrument through a shaft delivery lumen comprises pushing a portion of the medical instrument against the sidewall of the shaft to cause separation of the sidewall along the weakened portion.

Example 80: The method of any example herein, in particular example 79, wherein pushing the portion of the medical instrument against the sidewall of the shaft comprises engaging a distal end portion of the medical instrument with the sidewall of the shaft to cause separation of the sidewall along the weakened portion.

The above method(s) can be performed on a living animal or on a simulation, such as on a cadaver, cadaver heart, anthropomorphic ghost, simulator (e.g., with body parts, heart, tissue, etc. being simulated).

Depending on the example, certain acts, events, or functions of any of the processes or algorithms described herein can be performed in a different sequence, may be added, merged, or left out altogether. Thus, in certain examples, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y and at least one of Z to each be present.

It should be appreciated that in the above description of examples, various features are sometimes grouped together in a single example, Figure, or description thereof for the purpose of streamlining the disclosure and aiding in the understanding of one or more of the various aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the subject matter herein disclosed and claimed below should not be limited by the particular examples described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which examples belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A medical access system comprising:
an expandable access sheath comprising:
a hub, and
a shaft extending distally from the hub, the shaft having an expandable distal end portion configured to be in an open state and a closed state and a side shaft opening on the expandable distal end portion in fluid communication with a shaft delivery lumen extending through the shaft; and
a cord comprising:
a first cord tail portion configured to pass through the side shaft opening,
a second cord tail portion configured to pass through the side shaft opening, and
a medial cord portion between the first and second cord tail portions, the medial cord portion being configured to be disposed around a circumferential portion of the expandable distal end portion in the closed state.

2. The system of claim 1, wherein:
the side shaft opening comprises a first side shaft opening and a second side shaft opening; and
the first and the second cord tail portions are configured to extend through a respective one of the first and second side shaft openings and extend proximally along a portion of the shaft delivery lumen.

3. The system of claim 1, wherein the cord is configured to be removed from the expandable access sheath to allow the expandable distal end portion to assume the open state.

4. The system of claim 1, further comprising an introducer, the introducer comprising:
a central lumen extending through at least a portion of the introducer; and
a side introducer opening on a distal portion thereof, the side introducer opening in fluid communication with the central lumen, and
while the expandable distal end portion is in the closed state, at least a portion of the introducer being configured to be slidably received within the shaft delivery lumen, and the side introducer opening being configured to be within the shaft delivery lumen, and the first and second cord tail portions being configured to extend through the side shaft opening and the side introducer opening and along at least a portion of the central lumen of the introducer.

5. The system of claim 4, wherein the side shaft opening of the expandable distal end portion in the closed state is configured to be aligned with the side introducer opening.

6. The system of claim 4, wherein:
the side shaft opening comprises a first side shaft opening and a second side shaft opening;
the side introducer opening comprises a first side introducer opening and a second side introducer opening; and
the first and the second cord tail portions are configured to extend through a respective one of the first and second side shaft openings and a respective one of the first and second side introducer openings.

7. The system of claim 4, wherein:
the introducer is configured to be translated proximally relative to the expandable access sheath to withdraw the introducer from within the shaft delivery lumen; and
the first and second side introducer openings are configured to slide proximally along at least a portion of the first and second cord tail portions extending through the shaft delivery lumen as the introducer is translated proximally.

8. The system of claim 4, wherein the cord is configured to be removed from around the expandable access sheath and the central lumen of the introducer while the at least a portion of the introducer is received within the shaft delivery lumen to allow the expandable distal end portion to assume the open state.

9. The system of claim 1, wherein a portion of at least one of the first tail cord portion and the second tail cord portion is configured to be positioned around a portion of the circumferential portion of the expandable distal end portion to allow the expandable distal end portion to transform to the open state from the closed state.

10. The system of claim 1, wherein a proximal portion of the first tail cord portion and a proximal portion of the second tail cord portion are configured to be secured to the hub of the expandable access sheath to maintain the expandable distal end portion in the closed state or the open state.

11. The system of claim 1, wherein the first and second cord tail portions are disposed along the shaft delivery lumen proximal of the side shaft opening.

12. The system of claim 11, wherein respective portions of the first and second cord tail portions extend proximally from the shaft delivery lumen into a hub delivery lumen of the hub.

13. The system of claim 11, wherein portions of the first and second cord tail portions extend proximally of a proximal end of the hub.

14. The system of claim 1, wherein the first and second cord tail portions assume a tensioned state to transform the expandable distal end portion from the open state to the closed state.

15. The system of claim 1, wherein the circumferential portion of the expandable distal end portion is a medial portion along a longitudinal dimension of the expandable distal end portion.

16. An expandable access sheath comprising:
a hub; and
a shaft extending distally from the hub, the shaft comprising an expandable distal end portion and the expandable distal end portion comprising a circumferential portion having a weakened joint,
the circumferential portion being configured to be circumferentially disposed around a portion of the expandable distal end portion in a closed state, and
the circumferential portion being configured to separate along the weakened joint to allow the expandable distal end portion to assume an open state.

17. The sheath of claim 16, wherein:
the circumferential portion comprises a metal ring comprising the weakened joint; and
the metal ring is configured to separate along the weakened joint to allow the expandable distal end portion to assume the open state.

18. The sheath of claim 16, wherein:
the circumferential portion comprises an elastic ring comprising a weakened joint; and
the elastic ring is configured to stretch and separate along the weakened joint to allow the expandable distal end portion to assume the open state.

19. The sheath of claim 16, wherein the expandable distal end portion comprises a plurality of pleats and wherein:
the plurality of pleats is configured to be in a folded configuration to provide the expandable distal end portion in the closed state; and
the plurality of pleats is configured to be in an unfolded configuration to provide the expandable distal end portion in the open state.
